SEQUENCE LISTING (2 sequences)

SEQ ID NO: 1 - DNA, 792 bp, synthetic construct
  source 1..792: mol_type = other DNA
atggactggacctggatcctgttcctggtggccgccgccaccagagtgcacagcttccaggacccccaggagagcggcagaaagctgccccagctgtgcaccgagctgcagaccaccatccacgacatcatcctggagtgcgtgtactgcaagcagcagctgctgagaagagaggtgtacgacagagacctgtgcatcgtgtacagagacggcaacccctacgccgtgtgcgacaagtgcctgaagttctacagcaagatcagcgagtacagacactactgctacagcctgtacggcaccaccctggagcagcagtacaacaagcccctgtgcgacctgctgatcagatgcatcaactgccagaagcccctgcagagacacctggacaagaagcagagattccacaacatcagaggcagatggaccggcagatgcatgagctgctgcagaagcagcagaaccagaagagagacccagctgagaggcagaaagagaagaagccacggcgacacccccaccctgcacgagtacatgctggacctgcagcccgagaccaccgacctgtacggctacggccagctgaacgacagcagcgaggaggaggacgagatcgacggccccgccggccaggccgagcccgacagagcccactacaacatcgtgaccttctgctgcaagtgcgacagcaccctgagactgtgcgtgcagagcacccacgtggacatcagaaccctggaggacctgctgatgggcaccctgggcatcgtgtgccccatctgcagccagaagccc

SEQ ID NO: 2 - DNA, 792 bp, synthetic construct
  source 1..792: mol_type = other DNA
atggactggacctggatcctgttcctggtggccgccgccacacgggtgcacagcttccaggacccccaggagagcggcagaaagctgcctcagctgtgtaccgagctgcagaccaccatccacgacatcatcctggagtgtgtgtactgtaagcagcagctgctgaggagagaggtgtacgaccgggacctgtgtatcgtgtacagggacggcaatccctacgccgtgtgtgacaagtgcctgaagttctacagcaagatcagcgagtaccggcactactgctacagcctgtacggcaccaccctggagcagcagtacaacaagcccctgtgtgacctgctgatccggtgtatcaactgccagaagcccctgcagagacacctggacaagaagcagcggttccacaacatcaggggcagatggaccggcagatgtatgagctgctgccggagcagcagaaccagaagggagacccagctgagaggccggaagagaagaagccacggcgatacccccaccctgcacgagtacatgctggacctgcagcctgagaccaccgatctgtacggctacggccagctgaatgacagcagcgaggaggaggatgagatcgacggccctgccggccaggccgagcccgacagagcccactacaacatcgtgaccttttgctgtaagtgtgacagcaccctgagactgtgcgtgcagagcacccacgtggacatcagaaccctggaggatctgctgatgggcaccctgggcatcgtgtgtcccatctgctcccagaaaccc

(19) United States
(12) Patent Application Publication
(10) Pub. No.: US 2023/0250127 A1
(43) Pub. Date: Aug. 10, 2023

(54) MODIFIED mRNA 5'-CAP ANALOGS

(71) Applicant: Nanjing GeneLeap Biotechnology Co., Ltd., Nanjing (CN)

(72) Inventors: Ekambareswara Kandimalla, Hopkinton, MA (US); Jun Jiang, Westwood, MA (US); Lakshmi Bhagat, Framingham, MA (US); Sheng Bi, Nanjing (CN); Xin Xu, Nanjing (CN); Pengfei Li, Nanjing (CN); Praveen Pogula, Hopkinton, MA (US)

(21) Appl. No.: 18/057,714

(22) Filed: Nov. 21, 2022

Related U.S. Application Data
(60) Provisional application No. 63/283,046, filed on Nov. 24, 2021.

(51) Int. Cl. C07H 21/02; C12P 19/34; C12N 15/11

(52) U.S. Cl. CPC C07H 21/02; C12N 15/11; C12P 19/34

(57) ABSTRACT

The invention provides 5′-cap analogs, which can improve transcription, mRNA stability, and mRNA translation efficiency and durability. This invention also relates to methods useful for preparing cap analogs and using mRNA species containing such analogs, as well as kits containing the novel cap analogs.

This application claims the benefit of U.S. Provisional Application No. 63/283,046, filed on Nov. 24, 2021.

### REFERENCE TO AN ELECTRONIC SEQUENCE LISTING

The contents of the electronic sequence listing (690229_402_Sequence_Listing.xml; Size: 4,371 bytes; and Date of Creation: Nov. 21, 2022) is herein incorporated by reference in its entirety.

### TECHNICAL FIELD

This invention is directed to modification of the 5′ cap structure of RNA using nucleic acid complementary to the 5′ end of a messenger RNA to effect alteration of the 5′ cap structure of the RNA, thereby modulating its function. Thus, the invention generally relates to gene expression in animal cells, and to protein expression in particular.

### BACKGROUND

RNA degradation is a key process in the regulation of gene expression. Any functional mRNA, including mRNA for therapeutics, must be protected from the RNA degradation machinery. Yet, the protection in nature is temporary, and could be improved by suitable chemical modifications. In recent years, in vitro transcribed (IVT) mRNA has emerged as a promising route for therapeutic gene synthesis. Since proper mRNA function is dependent on its structural regulatory elements, including 5′-cap and polyA modifications at the 3′-end of an mRNA, these elements play a critical role in mRNA stability. Therefore, rational modifications of 5′-cap structure can improve the potency of mRNA therapeutics through improving RNA stability and facilitating its cellular and in vivo translation (Nature Reviews Drug Discovery, Vol. 17, 261).

In eukaryotes, 7-methylguanosine 5′-cap on mRNA is crucial for efficient recognition by protein synthesis machinery, e.g. ribosomes, in vitro and in vivo. Among mRNA manufacturing processes, a co-transcriptional method is more favored for in vitro transcription than a post-transcriptional 5′-cap method due to its simplicity and efficiency (Genes and Development, Vol. 20, 1838). To be suitable for co-transcription, 7-methylguanosine containing di- and trinucleotides with 5′-5′-triphosphate linker, known as cap-0, cap-1, ARCA and CleanCap have been developed and are currently used in approved mRNA COVID-19 vaccines and other multiple mRNAs that are currently in clinical trials (Nature, Vol. 596, 109). Published literature on 5′-cap analogs mainly rely on 7-methylguanosine (7mG) and its 2′- and 3′-substituents (US10925935B1, US9295717B2), phosphate replacement in the inter-nucleotide linkers (US10570388B2), such as substitution of a non-bridging oxygen with a sulphur moiety to give a phosphorothioate linkage (US8153773B2), or extended nucleotides such as in CleanCap® (US10519189B2). However, there is a need for i) improving capping efficiency in IVT, ii) increasing resistance against decapping machinery, and iii) improving binding to T7 or other polymerases, via using non-natural nucleoside modifications to replace 7 mG or triphosphate modifications that have not been explored. Therefore, we designed, synthesized, and evaluated a series of novel 5′-cap structures for mRNA therapeutic programs for a number of properties: stability, transcription, and /or translation efficiency and durability.

### BRIEF SUMMARY

The present disclosure provides mRNA 5′ cap analogs and methods of making and using them. The present disclosure also provides mRNA containing the 5′ cap analogs.

The 5′-cap analogs of the invention can improve transcription, mRNA stability, and mRNA translation efficiency and durability.

In one aspect, the present disclosure provides a compound of formula (I) below or a stereoisomer, tautomer or salt thereof:

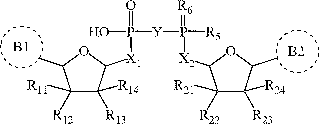

wherein:
rings B1 and B2 are each independently a nucleobase or a modified nucleobase;
X₁ and X₂ independently are —(C(R₁)₂)ₘ—, —O—(C(R₁)₂)ₘ —, — (C(R₁)₂)ₘ O—, —CH═CH—(C(R₁)₂)ₘ—, —(C(R₁)₂)ₘ—CH═CH—; in which m is an integer from 0-6;
Y is —(R₂—P(O)—R₂)ₚ—, in which p is 0, 1, or 2;
R₁₁, R₁₂, R₂₃ and R₂₄ independently is H, halogen, OR₃ or OR₄;
R₁₃, R₁₄, R₂₁, and R₂₂ independently is H, halogen, OR₃, OR₄, or LNA;
each R₁ independently is H, halogen, or C₁₋₃ alkyl;
each R₂ independently is O, S, C₁₋₆ alkyl, in which C₁₋₆ alkyl is optionally substituted with one or more of halogen, OH;
each R₃ independently is H, C₁₋₆ alkyl, C₂₋₆ alkenyl, C₂₋₆ alkynyl, in which C₁₋₆ alkyl, C₂₋₆ alkenyl, C₂₋₆ alkynyl is optionally substituted with one or more of halogen, OH and C₁₋₆ alkoxyl;
each R₄ independently is

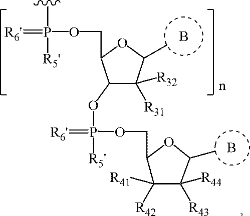

wherein, each B independently is a natural nucleobase or a modified nucleobase and may be the same or different;
R₃₁, R₃₂, R₄₃, and R₄₄ are each independently H, halogen, or OR₄′;
R₄₁, and R₄₂ are each independently H, halogen, OR₄′, LNA;
each R₄′ independently is H, C₁₋₆ alkyl, C₂₋₆ alkenyl, C₂₋₆ alkynyl, in which C₁₋₆ alkyl, C₂₋ 6 alkenyl, C₂₋₆ alkynyl is optionally substituted with one or more of halogen, OH and C₁₋₆ alkoxyl;
R₅ independently is OH or SH;
R₆ independently is O or S;
each R₅′ independently is OH or SH;
each R₆′ independently is O or S;
n is an integer selected from 0-2.

In another aspect, the present disclosure provides a compound of formula (II) below or a stereoisomer, tautomer, deuterate or salt thereof:

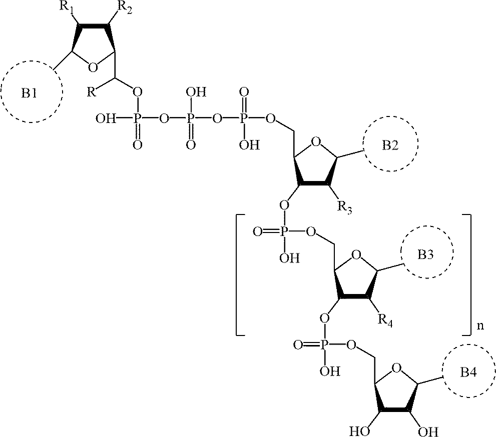

wherein:
ring B1, B2, B3 and B4 are each independently selected from a nucleobase and a modified nucleobase;
each R is independently selected from H and C₁₋₆ alkyl;
R₁ and R₂ are each independently selected from OR₅ and halogen;
each R₃ is independently selected from C₁₋₆ alkoxyl, halogen and LNA;
each R₄ is independently selected from halogen and LNA;
each R₅ is independently selected from H, C₁₋₆ alkyl, C₂₋₆ alkenyl and C₂₋₆ alkynyl, in which C₁₋₆ alkyl, C₂₋₆ alkenyl or C₂₋₆ alkynyl is optionally substituted with one or more of halogen, OH, and/or C₁₋₆ alkoxyl; and
n is an integer selected from 0-1.

The present disclosure also provides an RNA molecule whose 5′ end contains a compound of formula (I).

In another aspect, the RNA molecule of the present disclosure is an mRNA molecule.

In another aspect, the mRNA molecule of the present disclosure contains polynucleotide sequences encoding HPV E6-E7 antigen polypeptides such as those shown in SEQ ID NO: 1 or SEQ ID NO: 2.

In yet another aspect, the present disclosure provides methods of synthesizing the compound of formula (I).

In still another aspect, the present disclosure provides methods of synthesizing an RNA molecule (e.g., mRNA) in vitro. The method can include reacting unmodified or modified ATP, unmodified or modified CTP, unmodified or modified UTP, unmodified or modified GTP, a compound of formula (I) or a stereoisomer, tautomer or salt thereof, and a polynucleotide template, in the presence an RNA polymerase, under a condition conducive to transcription by the RNA polymerase of the polynucleotide template into one or more RNA copies, whereby at least some of the RNA copies incorporate the compound of formula (I) or a stereoisomer, tautomer or salt thereof to make an RNA molecule (e.g., mRNA).

In yet another aspect, the present disclosure provides a kit for capping an RNA transcript. The kit includes a compound of formula (I) and an RNA polymerase. The kit may also include one or more of RNA molecules, ribonuclease inhibitor, an enzyme buffer, and a nucleotide buffer.

Further, the compounds or methods described herein can be used for research (e.g., studying interaction of in vitro RNA transcript with certain enzymes) and other non-therapeutic purposes.

### BRIEF DESCRIPTION OF THE SEVERAL VIEWS OF THE DRAWINGS

FIG. 1 provides the expression of GLuc protein by mRNA containing different 5′-caps in HeLa cells at 24 hr after transfection. Activity is compared with New England Biolabs (NEB) and inhouse synthesized CapO and ARCA. Uncapped indicates mRNA without a cap structure, which does not show protein expression. The new compound 2 shows dose-dependent higher activity than NEB CapO and substantially similar activity compared with ARCA.

FIG. 2 shows the expression of GLuc protein by mRNA containing different 5′-caps in HeLa cells at 24 hr after transfection. Activity is compared with New England Biolabs (NEB) and inhouse synthesized CapO. Uncapped indicates mRNA without a cap structure, which does not show protein expression. The new compounds 11, 12 and 13 showed dose-dependent activity comparable to NEB CapO and compound 14 has no activity.

FIG. 3 provides the expression of GLuc protein by mRNA containing different 5′-caps in HeLa cells at 24 hr after transfection. Activity is compared with New England Biolabs (NEB) and inhouse synthesized CapO. Uncapped indicates mRNA without a cap structure, which does not show protein expression. mRNAs containing 5′-cap 26 and 27 showed dose-dependent protein expression. The new compound 26 shows greater activity than NEB cap0 and compound 27 shows slightly lower activity than NEB capO. Compound 31 does not have activity.

FIG. 4 shows the expression of GLuc protein by mRNA containing different 5′-caps in HeLa cells at 24 hr after transfection. Activity is compared with New England Biolabs (NEB) and inhouse synthesized CapO and ARCA. Uncapped indicates mRNA without a cap structure, which does not show protein expression. The mRNA containing 5′-cap 6 showed dose-dependent protein expression. The new compound 6 shows greater activity than NEB cap0 and the activity is comparable to ARCA.

FIG. 5 provides the expression of GLuc protein by mRNA containing different 5′-caps in HeLa cells at 24 hr after transfection. Activity is compared with New England Biolabs (NEB) and inhouse synthesized Cap0s. Uncapped indicates mRNA without a cap structure, which does not show protein expression. The mRNA containing 5′-cap 50 showed dose-dependent protein expression. The new compound 50 shows slightly lower activity than NEB cap0.

FIG. 6 shows the expression of GLuc protein by mRNA containing different 5′-caps in HeLa cells at 24 hr after transfection. Activity is compared with New England Biolabs (NEB) and inhouse synthesized CapO. Uncapped indicates mRNA without a cap structure and it does not show protein expression. The mRNA containing new 5′-caps 1, 17, 19 and 19A showed dose-dependent protein expression. The new compound 1 shows greater activity than cap0. Compound 17 has shown no activity while compounds 19 and 19A show slightly lower activity than NEB cap0. Inhouse cap0 shows higher activity than commercial NEB cap0.Analysis showed that the inhouse cap0 has greater purity than commercial cap0.

FIGS. 7A and 7B show the expression levels of Fluc protein by mRNAs containing different 5′caps in Hela cells at 24 h post-transfection. When the mRNAs were transfected using Lipofectamine MessengerMax (FIG. 7A), all caps showed a dose-dependent expression, and all in-house caps showed similar or significantly higher expression (especially at medium dose) than the control Cleancap AG; it is worth noting that GL-Cap5 exhibited a significantly higher expression level at different tested doses. When the mRNAs were transfected using in-house LNP formulation (FIG. 7B), the Caps expression was comparable to Cleancap AG (Trilink); at high and medium doses the expression of GL-Cap5 was significantly higher than all the other tested caps; at low doses, GL-Cap8 exhibited stronger expression than Cleancap AG (Trilink). Statistical significance: * p <0.05, ** p <0.01, *** p <0.001, **** p <0.0001.

FIG. 8 shows the expression levels of Fluc protein by mRNAs containing different 5′caps in Hela cells at 24 h post-transfection. The mRNAs were transfected using Lipofectamine MessengerMax. At high doses, GL-Cap6 showed a similar expression level as the control Cleancap AG, while the expression levels of GL-Cap2, GL-Cap3, GL-Cap4 and GL-Cap9 were slightly lower. At medium doses, the expression of GL-Cap3 and GL-Cap6 were slightly lower than Cleancap AG (without a statistical significance), while the expression of the other caps tested were significantly lower compared to Cleancap AG. At low doses, all the tested caps exhibited a similar expression level. Statistical significance: * p <0.05, ** p <0.01, *** p <0.001, **** p <0.0001.

FIG. 9 shows the expression levels of Fluc protein by mRNAs containing different 5′caps in Hela cells at 24 h post-transfection. The mRNAs were transfected using Lipofectamine MessengerMax and the expression level of GL-Cap5 was compared to that of another control cap Cleancap AG (3′-OM) (Trilink). At all doses, GL-Cap5 exhibited a similar expression level as Cleancap AG (3′-OM). Statistical significance: * p <0.05, ** p <0.01, *** p <0.001, **** p <0.0001.

FIG. 10 shows the in vivo expression levels of Fluc protein by mRNAs containing different 5′ caps (Experiment 1) as determined by in vivo imaging system after D-luciferin injection. Female C57BL/6 mice were intramuscularly injected with a single dose of mRNA-loaded LNPs and the Fluc expression levels were evaluated at 6 h, 24 h and 48 h. At both low and high dose regimens (upper and bottom panels, respectively), significantly higher Fluc expression levels were observed in mice injected with GL-Cap5-capped mRNAs compared to mice injected with Cleancap AG-mRNA. GL-Cap5 exhibited the strongest expression level among the tested caps, especially at 6 h and 24 h post-injection. Statistical significance: * p <0.05, ** p <0.01, *** p <0.001, **** p <0.0001.

FIG. 11 shows the expression levels of Fluc protein by mRNAs containing different 5′ caps (Experiment 1) in muscle tissue homogenates as determined by ex vivo Luciferase assay. At both low and high dose regimens (left and right panels, respectively), Fluc expression levels observed with GL-Cap5-mRNA were higher than with Cleancap AG-mRNA. The trend of a higher expression level of GL-Cap5 was observed in both regimens at different time points.

FIG. 12 shows the in vivo expression levels of Fluc protein by mRNAs containing different 5′ caps (Experiment 2) as determined by in vivo imaging system after D-luciferin injection. Female C57BL/6 mice were intramuscularly injected with a single dose of mRNA-loaded LNPs (0.1 mg/kg) and the Fluc protein expression levels were evaluated at 6 h, 24 h and 48 h to appraise the activity of the different caps. Six hours after the mRNA injection, significantly higher Fluc expression levels were observed in mice injected with GL-Cap5- and GL-Cap7-capped mRNAs in comparison to mice injected with Cleancap AG-mRNA; the protein expression in the GL-Cap1 group was also slightly higher than in the Cleancap AG group, but without statistical significance. At 24h and 48h post-injection, Fluc expression levels were slightly higher in all GL-Cap groups compared to the Cleancap AG control group. Statistical significance: * p <0.05, ** p <0.01, *** p <0.001, **** p <0.0001.

FIG. 13 shows the expression levels of Fluc protein by mRNAs containing different 5′ caps (Experiment 2) in liver and muscle (injection site) tissues of the treated mice as determined by in vivo imaging system after D-luciferin injection (top panels); and as determined by ex vivo Luciferase assay (bottom panels). Overall, all GL-Caps showed a comparable (e.g. GL-Cap5) to a better (e.g. GL-Cap1, 7 and 8) expression level than Cleancap AG. The highest expression levels were observed in the GL-Cap7 and GL-Cap8 groups. Statistical significance: * p <0.05, ** p <0.01, *** p <0.001, **** p <0.0001.

FIG. 14 shows the tumor growth curves of the synthesized 5′-cap structures on C3 Tumor Model. Data is presented as mean±SEM.

FIG. 15 shows the tumor growth curves of the synthesized 5′-cap structures on C3 Tumor Model. Data is presented as mean±SEM.

FIGS. 16A and 16B show the survival of the synthesized 5′-cap structures capped IL12 mRNA dose titration on MC38 Tumor Model. In FIG. 16A, data is presented as mean±SEM, and in FIG. 16B, data is presented as Kaplan-Meier curves.

FIG. 17 shows the protein expression as evaluated by intracellular staining 24 h after transfection of GL2288 mRNA in to Hela cells using Lipofectamine MessengerMax.

FIG. 18 shows the protein expression as evaluated by intracellular staining 24 h after transfection of GL2288 mRNA in to Hela cells using in-house LNP formulation. Statistical significance: * p <0.05, ** p <0.01, *** p <0.001, **** p <0.0001.

FIG. 19 shows the evaluation of the specific T cell immune response induced by GL-Cap7-GL2288 and CleanCap-GL2288 mRNA in mice.

### DETAILED DESCRIPTION

mRNA consists of an open reading frame (ORF) flanked by the 5′- and 3′- untranslated region (5′UTR, 3′UTR), a poly-adenosine monophosphate tail (poly A) and an inverted N7-methylguanosine-containing cap structure. It is both chemically and enzymatically less stable than the corresponding DNA, hence the protein production subsequent to the ribosomal recruitment of the mRNA is temporary. In addition, the mRNA must be present in a so-called “closed loop” conformation for production of the target protein. While part of the active closed-loop conformation, the mRNA makes contact with the ribosomal machinery through the cap that binds to the eukaryotic initiation factor 4E (eIF4E) and the polyA tail attached through the polyA-binding protein (PABP). The eIF4E and PABP are connected through a skeletal protein eIF4G closing the active loop. Disruption of the mRNA circularized form leads to cessation of protein production and eventually enzymatic degradation of the mRNA itself chiefly by action of the de-capping enzyme system DCP1/2 and or through a poly-A ribonuclease (PARN) mediated de-adenylation. See, e.g., Richard J. Jackson et al., “The mechanism of eukaryotic translation initiation and principles of its regulation”, Molecular Cell Biology, vol. 1 10, 1 13-127, 2010.

The cap-structure is a crucial feature of all eukaryotic mRNAs. It is recognized by the ribosomal complex through the eukaryotic initiation factor 4E (eIF4E). mRNAs lacking the 5′- cap terminus are not recognized by the translational machinery and are incapable of producing the target protein (see, e.g., Colin Echeverria Aitken, Jon R Lorsch: “A mechanistic overview of translation initiation in eukaryotes”, Nature Structural and Molecular Biology, vol. 16, no. 6, 568-576, 2012.)

The crude messenger RNA produced during the transcription process (“primary transcript”) is terminated by a 5′-triphosphate, which is converted to the respective 5′-diphosphate by the action of the enzyme RNA-triphosphatase. Then a guanylyl-transferase attaches the terminal inverted guanosine monophosphate to the 5′-terminus, and an N7MTase- mediated N7-methylation of the terminal, inverted guanosine, completes the capping process.

The wild-type 5′-cap structure is vulnerable to enzymatic degradation, which is part of the regulation mechanism controlling protein expression. According to this mechanism, the enzymatic system DCP1/2 performs a pyrophosphate hydrolysis between the second and the third phosphate groups of the cap structure, removing the N7-methylated guanosine diphosphate moiety leaving behind an mRNA terminated in a 5′-monophosphate group. This in turn is quite vulnerable to exonuclease cleavage and will lead to rapid decay of the remaining oligomer. See, e.g., R. Parker, H. Song: “The Enzymes and Control of Eukaryotic Turnover”, Nature Structural & Molecular Biology, vol. 11, 121 -127, 2004.

The present disclosure provides novel mRNA cap analogs, synthetic methods for making these cap analogs, and uses thereof. The present disclosure also provides new RNA molecules (e.g., mRNAs) incorporating the cap analogs disclosed herein which impart properties that are advantageous to therapeutic development.

Most mRNA therapeutic applications or clinical candidates utilize 7mG with or without 3′-substitutions as the head nucleoside group.

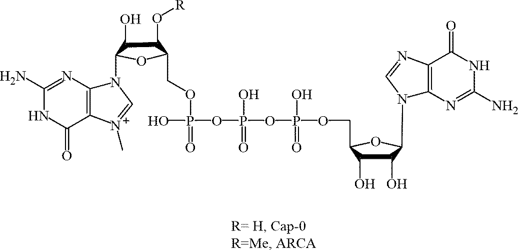

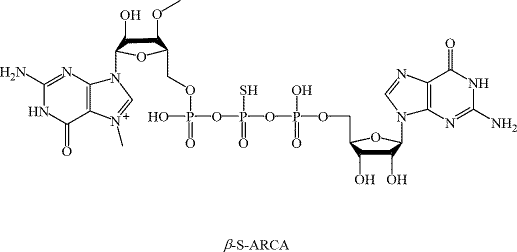

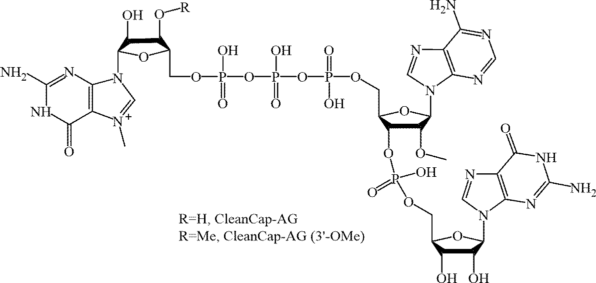

We designed and synthesized 5′-caps with 7 mG analogs and evaluated them in in vitro biological studies to select an optimal head nucleoside group. Once potent head nucleoside groups were identified, we coupled them to various tail nucleoside groups with unmodified or modified triphosphate linkers for improved binding to promoters to increase in vitro transcription yield and translation efficiency.

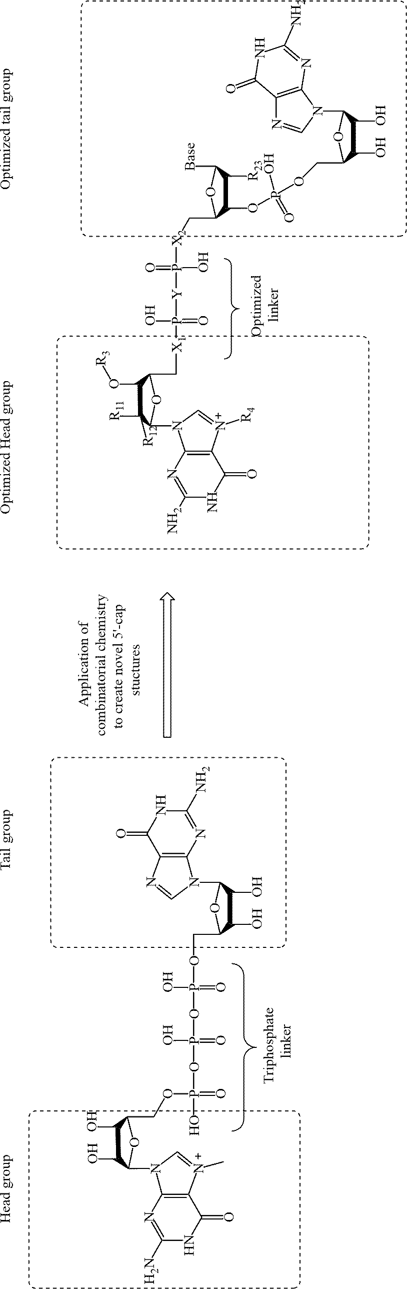

### 5′-Cap Analogs

In one aspect, the present disclosure features a compound of formula (I) below or a stereoisomer, tautomer or salt thereof:

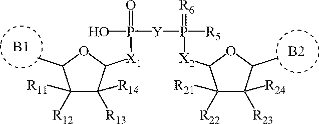

wherein:
each of ring B1 and B2 independently is a nucleobase or a modified nucleobase;
each of X₁ and X₂ independently is —(C(R₁)₂)ₘ—, —O—(C(R₁)₂)ₘ —, — (C(R₁)₂)ₘ O—, —CH═CH—(C(R₁)₂)ₘ—, —(C(R₁)₂)ₘ—CH═CH—; in which m is an integer from 0-6;
Y is —(R₂—P(O)—R₂)ₚ—, in which p is 0, 1, or 2;
each of R₁₁, R₁₂, R₂₃ and R₂₄ independently is H, halogen, OR₃ or OR₄;
each of R₁₃, R₁₄, R₂₁, and R₂₂ independently is H, halogen, OR₃, OR₄, or LNA;
each R₁ independently is H, halogen, or C₁₋₃ alkyl;
each R₂ independently is O, S, C₁₋₆ alkyl, in which C₁₋₆ alkyl is optionally substituted with one or more of halogen, OH;
each R₃ independently is H, C₁₋₆ alkyl, C₂₋₆ alkenyl, C₂₋₆ alkynyl, in which C₁₋₆ alkyl, C₂₋₆ alkenyl, C₂₋₆ alkynyl is optionally substituted with one or more of halogen, OH and C₁₋₆ alkoxyl;
each R₄ independently is

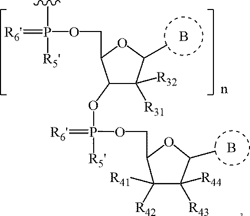

wherein, each B are the same or different, and independently is a natural nucleobase or a modified nucleobase;
R₃₁, R₃₂, R₄₃, and R₄₄ is independently is H, halogen, or OR₄′;
R₄₁, and R₄₂ independently is H, halogen, OR₄′, LNA;
each R₄′ independently is H, C₁₋₆ alkyl, C₂₋₆ alkenyl, C₂₋₆ alkynyl, in which C₁₋₆ alkyl, C₂₋ 6 alkenyl, C₂₋₆ alkynyl is optionally substituted with one or more of halogen, OH and C₁₋₆ alkoxyl;
R₅ independently is OH or SH;
R₆ independently is O or S;
each R₅′ independently is OH or SH;
each R₆′ independently is O or S;
n is an integer selected from 0-2.

In certain embodiments, ring B1, B2 and each ring B independently is selected from

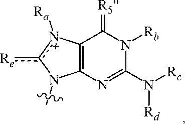

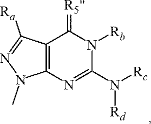

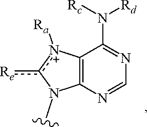

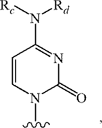

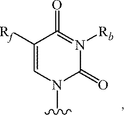

in which each Rₐ independently is H, C₁₋₆ alkyl, C₂₋₆ alkenyl, C₂₋₆ alkynyl, each of which is optionally substituted with one or more substituents selected from the group consisting of halogen, C₁₋₆ alkoxyl, C₃₋₈ cycloalkyl, or C₃₋₈ heterocycloalkyl;
each R_{b} independently is H, NH₂, or C₁₋₆ alkyl;
each R_{c} independently is H, C₁₋₆ alkyl, or an amine protecting group;
each R_{d} independently is H, C₁₋₆ alkyl, or an amine protecting group;
each Rₑ independently is H, O, S;
each Rf independently is H, NH₂, or C₁₋₆ alkyl;
each R₅″ independently is O, S.

For example, ring B1, ring B2 and each ring B independently is a nucleobase or a modified nucleobase, wherein said nucleobase is selected from adenine (A), cytosine (C), guanine (G), thymine (T), or uracil (U), or said modified nucleobase is selected from hypoxanthine, xanthine, 7-methylguanine, 7-ethylguanine, 7-isopropylguanosine, 7-cyclopropylguanosine, 7-methyl-N1-methylguanosine, 7-(oxetanyl-methyl)guanosine, 7-(oxetanyl-ethyl)guanosine, 6-thioguanosine, 7-methyl-(6-thio)guanosine, 7-deaza-8-aza-guanosine, 7-methyl-7-deaza-8-aza-guanosine, 6-methyladenosine, 7-methyladenosine, 7-methylxanthine, 7-methyl hypoxanthine, 5,6-dihydrouracil, 5-methylcytosine, or dihydrouridine.

For example, the “amine protecting group” includes but is not limited to fluorenylmethyloxycarbonyl (“Fmoc”), carboxybenzyl (“Cbz”), tert-butyloxycarbonyl (“BOC”), dimethoxybenzyl (“DMB”), acetyl (“Ac”), trifluoroacetyl, phthalimide, benzyl (“Bn”), Trityl (triphenylmethyl, Tr), benzylideneamine, or Tosyl (Ts).

In certain embodiments, each of X₁ and X₂ independently is —(C(R₁)₂)ₘ—, —O—(C(R₁)₂)ₘ —, —(C(R₁)₂)ₘ—O—, —CH═CH—; wherein each R₁ independently is H, F, Cl, Br, I or C₁₋₃ alkyl, wherein m is an integer selected from 0-6.

For example, X₁ and X₂ independently is —CH₂—O—, —O—CH₂—, —CH₂—CH₂—, —O—CH(CH₃)—, —CH(CH₃)—O—, or —CH═CH—.

In certain embodiments, Y is —(R₂—P(O)—R₂)ₚ—, wherein each R₂ independently is O, S, C₁₋₆ alkyl, in which C₁₋₆ alkyl is optionally substituted with one or more of halogen or OH; wherein p is 0, 1, or 2.

For example, Y is selected from

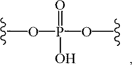

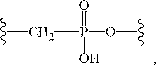

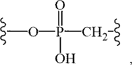

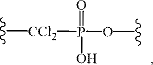

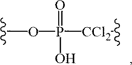

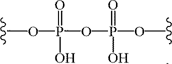

In certain embodiments, each of R₁₁, R₁₂, R₂₃ and R₂₄ independently is H, F, Cl, Br, I, or OR₃; each of R₁₃, R₁₄, R₂₁, and R₂₂ independently is H, F, Cl, Br, I, OR₃, or LNA; each R₃ independently is H, C₁₋₆ alkyl, C₂₋₆ alkenyl, or C₂₋₆ alkynyl, in which C₁₋₆ alkyl, C₂₋₆ alkenyl, or C₂₋₆ alkynyl are optionally substituted with one or more of F, Cl, Br, I, OH and C₁₋₆ alkoxyl;

In certain embodiments, each of R₁₁, R₁₂, R₂₃ and R₂₄ independently is H, F, Cl, Br, I or OR₃; each of R₁₃ and R₁₄ independently is H, F, Cl, Br, I OR₃, or LNA; any one of R₂₁ and R₂₂ independently is H, F, Cl, Br, I or OR₃, and the other is OR₄, wherein,
each R₃ independently is H, C₁₋₆ alkyl, C₂₋₆ alkenyl, C₂₋₆ alkynyl, in which C₁₋₆ alkyl, C₂₋₆ alkenyl, C₂₋₆ alkynyl is optionally substituted with one or more of F, Cl, Br, I, OH and C₁₋₆ alkoxyl;
R₄ independently is

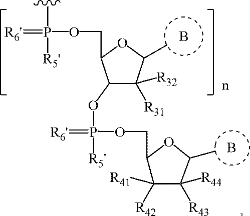

wherein, each B are the same or different, and is independently a nucleobase or a modified nucleobase;
Each of R₃₁, R₃₂, R₄₃, and R₄₄ is independently H, F, Cl, Br, I, or OR₄′;
each R₄₁ and R₄₂ independently is H, F, Cl, Br, I, OR₄′, or LNA;
each R₄′ independently is H, C₁₋₆ alkyl, C₂₋₆ alkenyl, C₂₋₆ alkynyl, in which C₁₋₆ alkyl, C₂₋ 6 alkenyl, C₂₋₆ alkynyl is optionally substituted with one or more of F, Cl, Br, I, OH and C₁₋₆ alkoxyl;
each R₅′ independently is OH or SH;
each R₆′ independently is O or S;
n is 0, 1, or 2.

For example, ring B independently is

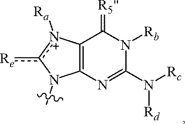

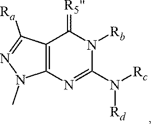

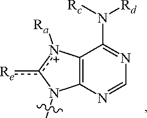

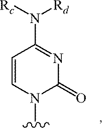

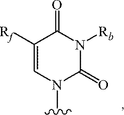

in which each Rₐ independently is H, C₁₋₆ alkyl, C₂₋₆ alkenyl, C₂₋₆ alkynyl, each of which is optionally substituted with one or more substituents selected from the group consisting of halogen, C₁₋₆ alkoxyl, C₃₋₈ cycloalkyl, or C₃₋₈ heterocycloalkyl;
each R_{b} independently is H, NH₂, or C₁₋₆ alkyl;
each R_{c} independently is H, C₁₋₆ alkyl, or an amine protecting group;
each R_{d} independently is H, C₁₋₆ alkyl, or an amine protecting group;
each Rₑ independently is H, O, or S;
each Rf independently is H, NH₂, or C₁₋₆ alkyl;
each R₅″ independently is O or S.

For example, ring B1, ring B2 and each ring B independently is a nucleobase or a modified nucleobase, said nucleobase is selected from adenine (A), cytosine (C), guanine (G), thymine (T), or uracil (U), or said modified nucleobase is selected from hypoxanthine, xanthine, 7-methylguanine, 7-ethylguanine, 7-isopropylguanosine, 7-cyclopropylguanosine, 7-methyl-N1-methylguanosine, 7-(oxetanyl-methyl)guanosine, 7-(oxetanyl-ethyl)guanosine, 6-thioguanosine, 7-methyl-(6-thio)guanosine, 7-deaza-8-aza-guanosine, 7-methyl-7-deaza-8-aza-guanosine, 6-methyladenosine, 7-methyladenosine, 7-methylxanthine, 7-methyl hypoxanthine, 5,6-dihydrouracil, 5-methylcytosine, or dihydrouridine.

In certain embodiments, each of R₁₁, R₁₂, R₂₃ and R₂₄ independently is H, F, Cl, Br, I or OR₃; each of R₁₃ and R₁₄ independently is H, F, Cl, Br, I, OR₃, or LNA; any one of R₂₁ and R₂₂ independently is H, F, Cl, Br, I or OR₃, wherein the other is OR₄, wherein,
each R₃ independently is H, C₁₋₆ alkyl, C₂₋₆ alkenyl, C₂₋₆ alkynyl, in which C₁₋₆ alkyl, C₂₋₆ alkenyl, C₂₋₆ alkynyl is optionally substituted with one or more of F, Cl, Br, I, OH and C₁₋₆ alkoxyl;
R₄ independently is selected from the group consisting of:

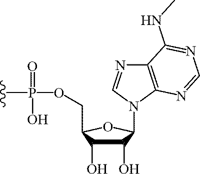

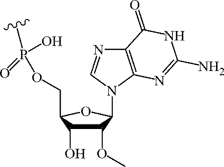

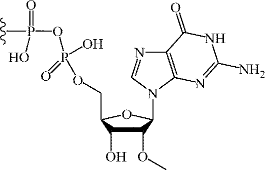

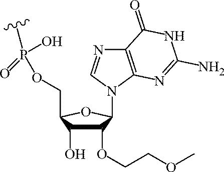

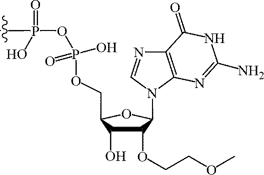

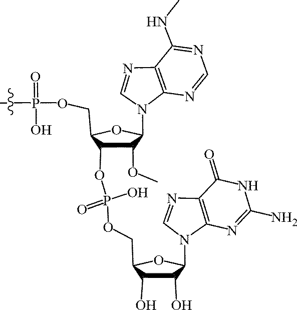

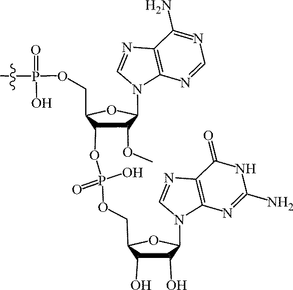

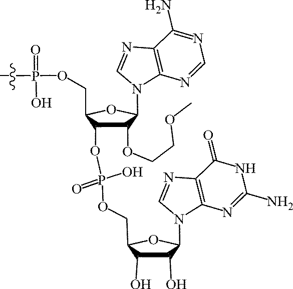

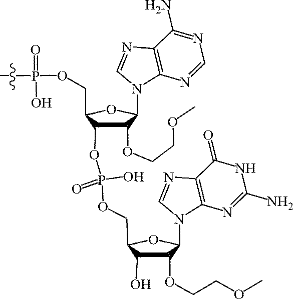

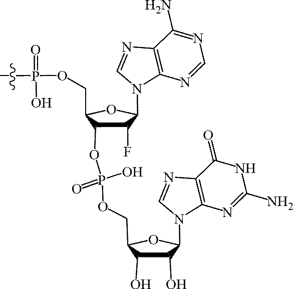

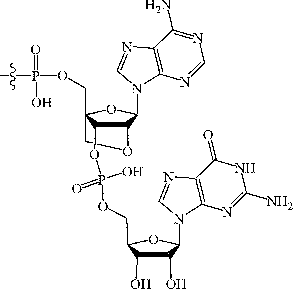

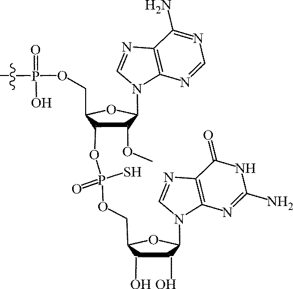

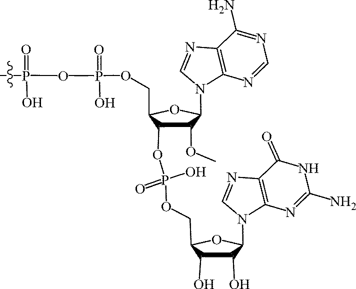

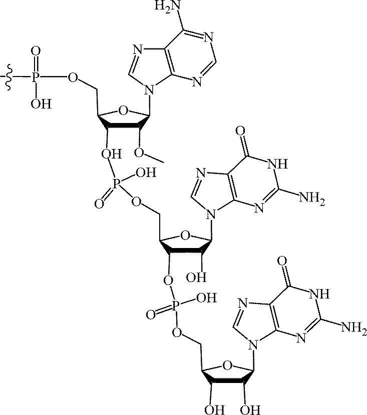

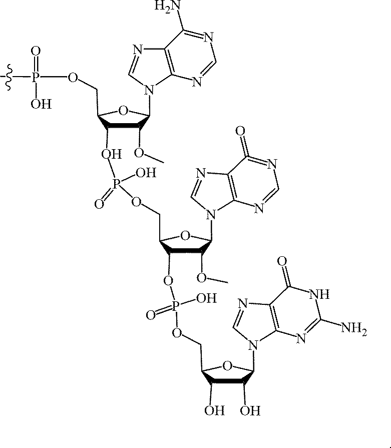

In another aspect, the present disclosure provides a compound of formula (Ia) or (Ib) below or a stereoisomer, tautomer or salt thereof:

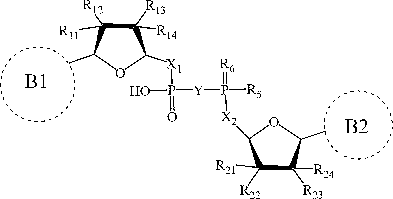

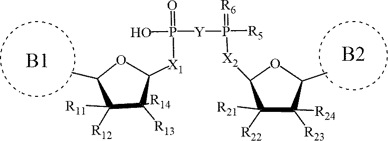

In formula (Ia) or (Ib), each of ring B1 and B2 independently is a nucleobase or a modified nucleobase;
X₁ and X₂ are independently selected from —(C(R₁)₂)ₘ—, —O—(C(R₁)₂)m —, — (C(R₁)₂)ₘ—O—, —CH═CH—(C(R₁)₂)m—, —(C(Rₗ)₂)ₘ—CH═CH—; in which m is an integer from 0-6;
Y is —(R₂—P(O)—R₂)ₚ—, in which p is 0, 1, or 2;
R₁₁, R₁₂, R₂₃ and R₂₄ are independently selected from H, halogen, OR₃ or OR₄;
R₁₃, R₁₄, R₂₁, and R₂₂ are independently selected from H, halogen, OR₃, OR₄, or LNA;
each R₁ independently is H, halogen, or C₁₋₃ alkyl;
each R₂ independently is O, S, or C₁₋₆ alkyl, in which C₁₋₆ alkyl is optionally substituted with one or more of halogen or OH;
each R₃ independently is H, C₁₋₆ alkyl, C₂₋₆ alkenyl, or C₂₋₆ alkynyl, in which C₁₋₆ alkyl, C₂₋₆ alkenyl, or C₂₋₆ alkynyl is optionally substituted with one or more of halogen, OH and C₁₋₆ alkoxyl;
each R₄ independently is

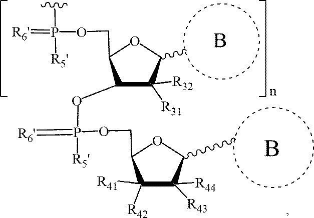

wherein, each B are the same or different and are independently selected from a natural nucleobase or a modified nucleobase;
R₃₁, R₃₂, R₄₃, and R₄₄ are independently selected from H, halogen, or OR₄′;
R₄₁ and R₄₂ are independently selected from H, halogen, OR₄′, or LNA;
each R₄′ independently is H, C₁₋₆ alkyl, C₂₋₆ alkenyl, or C₂₋₆ alkynyl, in which C₁₋₆ alkyl,
C₂₋₆ alkenyl, or C₂₋₆ alkynyl is optionally substituted with one or more of halogen, OH and C₁₋₆ alkoxyl;
Rs independently is OH or SH;
R₆ independently is O or S;
each R₅′ independently is OH or SH;
each R₆′ independently is O or S;
n is an integer selected from 0-2.

In certain embodiments, ring B1, B2 and each ring B independently is selected from:

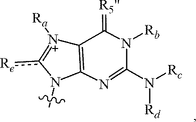

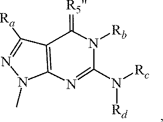

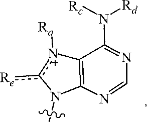

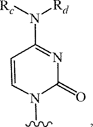

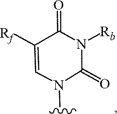

in which each Rₐ independently is H, C₁₋₆ alkyl, C₂₋₆ alkenyl, C₂₋₆ alkynyl, each of which is optionally substituted with one or more substituents selected from the group consisting of halogen, C₁₋₆ alkoxyl, C₃₋₈ cycloalkyl, or C₃₋₈ heterocycloalkyl;
each R_{b} independently is H, NH₂, or C₁₋₆ alkyl;
each R_{c} independently is H, C₁₋₆ alkyl, or an amine protecting group;
each R_{d} independently is H, C₁₋₆ alkyl, or an amine protecting group;
each Rₑ independently is H, O, or S;
each R_{f} independently is H, NH₂, or C₁₋₆ alkyl;
each R₅″ independently is O or S.

For example, ring B 1, ring B2 and each ring B independently is a nucleobase or a modified nucleobase, wherein said nucleobase is selected from adenine (A), cytosine (C), guanine (G), thymine (T), or uracil (U), or said modified nucleobase is selected from hypoxanthine, xanthine, 7-methylguanine, 7-ethylguanine, 7-isopropylguanosine, 7-cyclopropylguanosine, 7-methyl-N1-methylguanosine, 7-(oxetanyl-methyl)guanosine, 7-(oxetanyl-ethyl)guanosine, 6-thioguanosine, 7-methyl-(6-thio)guanosine, 7-deaza-8-aza-guanosine, 7-methyl-7-deaza-8-aza-guanosine, 6-methyladenosine, 7-methyladenosine, 7-methylxanthine, 7-methyl hypoxanthine, 5,6-dihydrouracil, 5-methylcytosine, or dihydrouridine.

For example, the “amine protecting group” includes but is not limited to fluorenylmethyloxycarbonyl (“Fmoc”), carboxybenzyl (“Cbz”), tert-butyloxycarbonyl (“BOC”), dimethoxybenzyl (“DMB”), acetyl (“Ac”), trifluoroacetyl, phthalimide, benzyl (“Bn”), Trityl (triphenylmethyl, Tr), benzylideneamine, or Tosyl (Ts).

In certain embodiments, X₁ and X₂ independently are selected from —(C(R₁)₂)ₘ—, —O—(C(R₁)₂)ₘ —, — (C(R₁)₂)ₘ—O—, or —CH═CH—; wherein each R₁ independently is H, F, Cl, Br, I, or C₁₋₃ alkyl, wherein m is an integer selected from 0-6.

For example, X₁ and X₂ independently are selected from —CH₂—O—, —O—CH₂—, —CH₂—CH₂—, —O—CH(CH₃)—, —CH(CH₃)—O—, or —CH═CH—.

In certain embodiments, Y is —(R₂—P(O)—R₂)ₚ—, wherein each R₂ independently is O, S, or C₁₋₆ alkyl, in which C₁₋₆ alkyl is optionally substituted with one or more of halogen or OH; and wherein p is 0, 1, or 2.

For example, Y is selected from

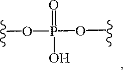

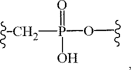

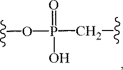

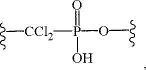

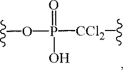

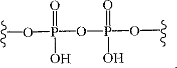

In certain embodiments, R₁₁, R₁₂, R₂₃ and R₂₄ are independently selected from H, F, Cl, Br, I, or OR₃; R₁₃, R₁₄, R₂₁, and R₂₂ are independently selected from H, F, Cl, Br, I, OR₃, or LNA; each R₃ independently is H, C₁₋₆ alkyl, C₂₋₆ alkenyl, or C₂₋₆ alkynyl, in which C₁₋₆ alkyl, C₂₋₆ alkenyl, or C₂₋₆ alkynyl is optionally substituted with one or more of F, Cl, Br, I, OH and C₁₋₆ alkoxyl.

In certain embodiments, R₁₁, R₁₂, R₂₃ and R₂₄ are independently selected from H, F, Cl, Br, I or OR₃; R₁₃ and R₁₄ are independently selected from H, F, Cl, Br, I, OR₃, or LNA; any one of R₂₁ and R₂₂ independently is H, F, Cl, Br, I or OR₃, and the other is OR₄, wherein,
each R₃ independently is H, C₁₋₆ alkyl, C₂₋₆ alkenyl, or C₂₋₆ alkynyl, in which C₁₋₆ alkyl, C₂₋₆ alkenyl, or C₂₋₆ alkynyl is optionally substituted with one or more of F, Cl, Br, I, OH and C₁₋₆ alkoxyl;
R₄ independently is

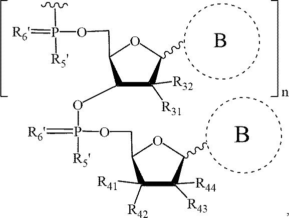

wherein, each B are the same or different, and independently is a nucleobase or a modified nucleobase;
R₃₁, R₃₂, R₄₃, and R₄₄ are independently H, F, Cl, Br, I, or OR₄′;
R₄₁ and R₄₂ are independently H, F, Cl, Br, I, OR₄′, or LNA;
each R₄′ independently is H, C₁₋₆ alkyl, C₂₋₆ alkenyl, or C₂₋₆ alkynyl, in which C₁₋₆ alkyl, C₂₋₆ alkenyl, or C₂₋₆ alkynyl is optionally substituted with one or more of F, Cl, Br, I, OH and C₁₋₆ alkoxyl;
each R₅′ independently is OH or SH;
each R₆′ independently is O or S; and
n is 0, 1, or 2.

For example, ring B independently is

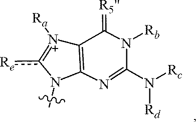

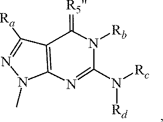

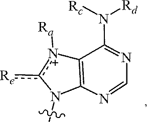

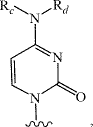

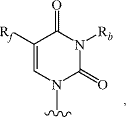

in which each Rₐ independently is H, C₁₋₆ alkyl, C₂₋₆ alkenyl, or C₂₋₆ alkynyl, each of which is optionally substituted with one or more substituents selected from the group consisting of halogen, C₁₋₆ alkoxyl, C₃₋₈ cycloalkyl, or C₃₋₈ heterocycloalkyl;
each R_{b} independently is H, NH₂, or C₁₋₆ alkyl;
each R_{c} independently is H, C₁₋₆ alkyl, or an amine protecting group;
each R_{d} independently is H, C₁₋₆ alkyl, or an amine protecting group;
each Rₑ independently is H, O, or S;
each R_{f} independently is H, NH₂, or C₁₋₆ alkyl;
each R₅′ independently is O or S.

For example, ring B1, ring B2 and each ring B independently is a nucleobase or a modified nucleobase, wherein said nucleobase is selected from adenine (A), cytosine (C), guanine (G), thymine (T), or uracil (U), or wherein said modified nucleobase is selected from hypoxanthine, xanthine, 7-methylguanine, 7-ethylguanine, 7-isopropylguanosine, 7-cyclopropylguanosine, 7-methyl-N1-methylguanosine, 7-(oxetanyl-methyl)guanosine, 7-(oxetanyl-ethyl)guanosine, 6-thioguanosine, 7-methyl-(6-thio)guanosine, 7-deaza-8-aza-guanosine, 7-methyl-7-deaza-8-aza-guanosine, 6-methyladenosine, 7-methyladenosine, 7-methylxanthine, 7-methyl hypoxanthine, 5,6-dihydrouracil, 5-methylcytosine, or dihydrouridine.

In certain embodiments, R₁₁, R₁₂, R₂₃ and R₂₄ independently are H, F, Cl, Br, I or OR₃; R₁₃ and R₁₄ independently are H, F, Cl, Br, I, OR₃, or LNA; any one of R₂₁ and R₂₂ independently is H, F, Cl, Br, I or OR₃, and the other is OR₄, wherein,
each R₃ independently is H, C₁₋₆ alkyl, C₂₋₆ alkenyl, or C₂₋₆ alkynyl, in which C₁₋₆ alkyl,
C₂₋₆ alkenyl, or C₂₋₆ alkynyl is optionally substituted with one or more of F, Cl, Br, I, OH and C₁₋₆ alkoxyl;
R₄ independently is selected from the group consisting of:

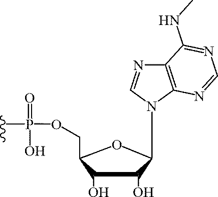

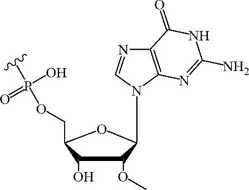

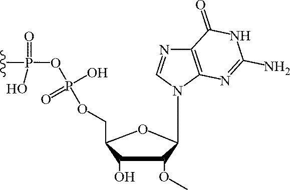

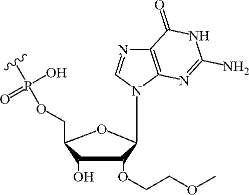

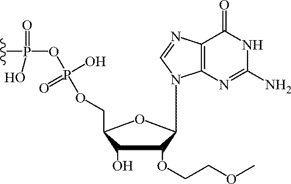

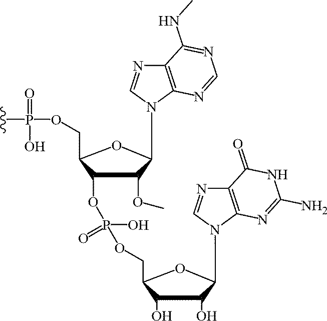

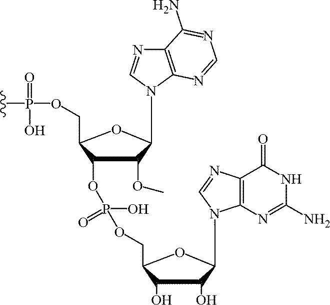

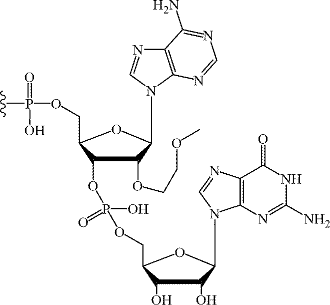

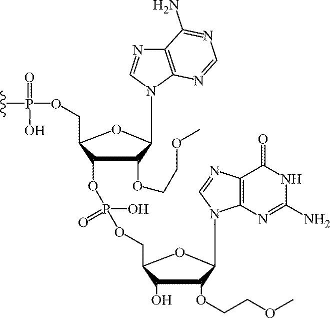

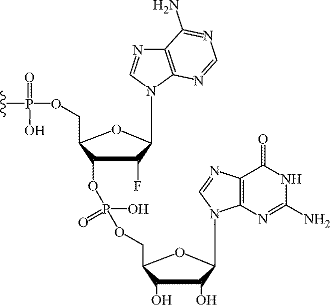

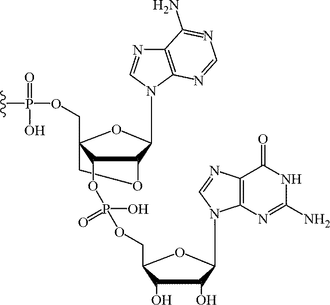

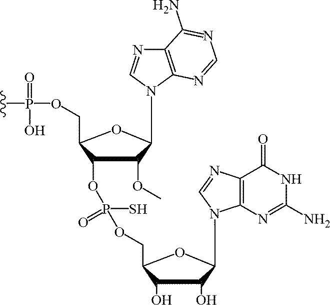

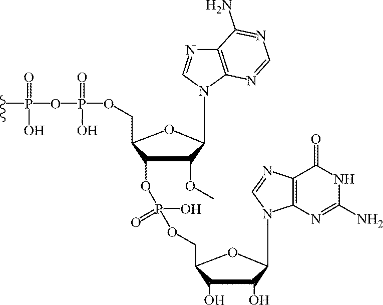

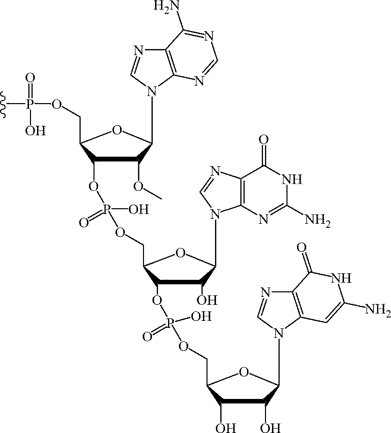

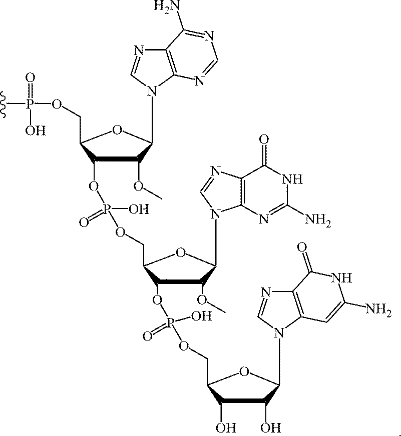

In another aspect, the present disclosure provides a compound of formula (Ia1), (Ia2), (Ia3) or (Ia4) below or a stereoisomer, tautomer or salt thereof:

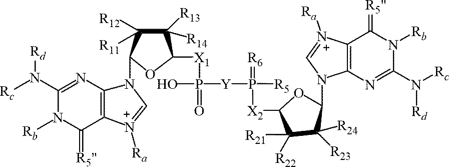

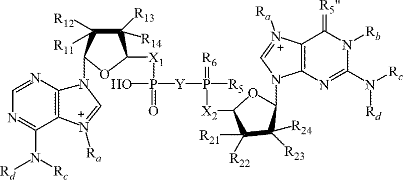

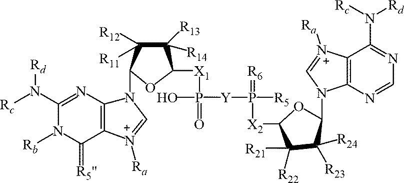

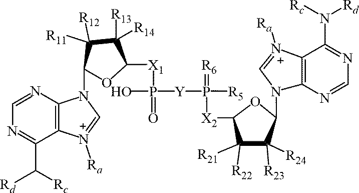

In certain embodiments, each Rₐ independently is H, C₁₋₆ alkyl, C₂₋₆ alkenyl, or C₂₋₆ alkynyl, each of which is optionally substituted with one or more substituents selected from the group consisting of halogen, C₁₋₆ alkoxyl, C₃₋₈ cycloalkyl, or C₃₋₈ heterocycloalkyl;
each R_{b} independently is H, NH₂, or C₁₋₆ alkyl;
each R_{c} independently is H, C₁₋₆ alkyl, or an amine protecting group;
each R_{d} independently is H, C₁₋₆ alkyl, or an amine protecting group;
each R₅″ independently is O or S.

For example, the “amine protecting group” includes but is not limited to fluorenylmethyloxycarbonyl (“Fmoc”), carboxybenzyl (“Cbz”), tert-butyloxycarbonyl (“BOC”), dimethoxybenzyl (“DMB”), acetyl (“Ac”), trifluoroacetyl, phthalimide, benzyl (“Bn”), Trityl (triphenylmethyl, Tr), benzylideneamine, or Tosyl (Ts).

In certain embodiments, X₁ and X₂ are independently selected from —(C(R₁)₂)ₘ—, —O—(C(R₁)₂)ₘ —, — (C(R₁)₂)ₘ—O—, or —CH═CH—; wherein each R₁ independently is H, F, Cl, Br, I or C₁₋₃ alkyl, wherein m is an integer selected from 0-6.

For example, each of X₁ and X₂ independently is —CH₂—O—, —O—CH₂—, —CH₂—CH₂—, —O—CH(CH₃)—, —CH(CH₃)—O—, or —CH═CH—.

In certain embodiments, Y is —(R₂—P(O)—R₂)ₚ—, wherein each R₂ independently is O, S, or C₁₋₆ alkyl, in which C₁₋₆ alkyl is optionally substituted with one or more of halogen or OH; and wherein p is 0, 1, or 2.

For example, Y is selected from

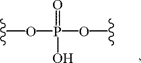

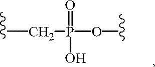

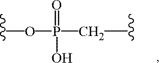

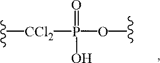

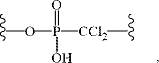

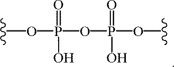

In certain embodiments, R₁₁, R₁₂, R₂₃ and R₂₄ independently is H, F, Cl, Br, I, or OR₃; R₁₃, R₁₄, R₂₁, and R₂₂ independently is H, F, Cl, Br, I, OR₃, or LNA; each R₃ independently is H, C₁₋₆ alkyl, C₂₋₆ alkenyl, or C₂₋₆ alkynyl, in which C₁₋₆ alkyl, C₂₋₆ alkenyl, or C₂₋₆ alkynyl is optionally substituted with one or more of F, Cl, Br, I, OH and C₁₋₆ alkoxyl;

In certain embodiments, R₁₁, R₁₂, R₂₃ and R₂₄ independently is H, F, Cl, Br, I or OR₃; R₁₃, and R₁₄, independently is H, F, Cl, Br, I, OR₃, or LNA; any one of R₂₁ and R₂₂ independently is H, F, Cl, Br, I or OR₃, and the other is OR₄, wherein,
each R₃ independently is H, C₁₋₆ alkyl, C₂₋₆ alkenyl, or C₂₋₆ alkynyl, in which C₁₋₆ alkyl, C₂₋₆ alkenyl, or C₂₋₆ alkynyl is optionally substituted with one or more of F, Cl, Br, I, OH, and C₁₋₆ alkoxyl;
R₄ independently is

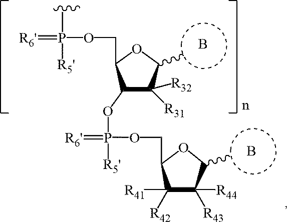

wherein,
each B are the same or different, and independently is selected from a nucleobase or a modified nucleobase, wherein said nucleobase is selected from adenine (A), cytosine (C), guanine (G), thymine (T), or uracil (U), or wherein said modified nucleobase is selected from hypoxanthine, xanthine, 7-methylguanine, 7-ethylguanine, 7-isopropylguanosine, 7-cyclopropylguanosine, 7-methyl-N1-methylguanosine, 7-(oxetanyl-methyl)guanosine, 7-(oxetanyl-ethyl)guanosine, 6-thioguanosine, 7-methyl-(6-thio)guanosine, 7-deaza-8-aza-guanosine, 7-methyl-7-deaza-8-aza-guanosine, 6-methyladenosine, 7-methyladenosine, 7-methylxanthine, 7-methyl hypoxanthine, 5,6-dihydrouracil, 5-methylcytosine, or dihydrouridine;
R₃₁, R₃₂, R₄₃, and R₄₄ are independently selected from H, F, Cl, Br, I, or OR₄′;
R₄₁ and R₄₂ are independently selected from H, F, Cl, Br, I, OR₄′, or LNA;
each R₄′ independently is selected from H, C₁₋₆ alkyl, C₂₋₆ alkenyl, or C₂₋₆ alkynyl, in which C₁₋₆ alkyl, C₂₋₆ alkenyl, or C₂₋₆ alkynyl is optionally substituted with one or more of F, Cl, Br, I, OH and C₁₋₆ alkoxyl;
each R₅′ independently is OH or SH;
each R₆′ independently is O or S;
n is 0, 1, or 2.

In certain embodiments, each R₄ is independently selected from the group consisting of:

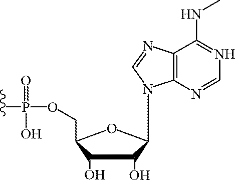

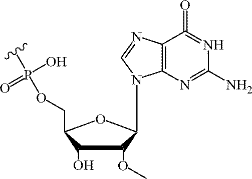

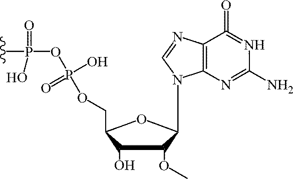

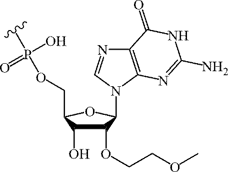

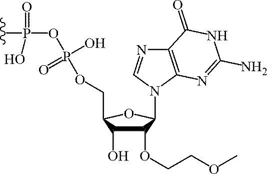

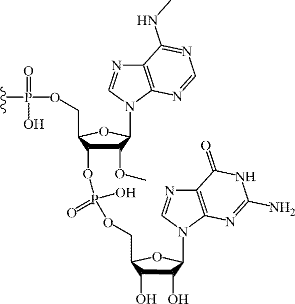

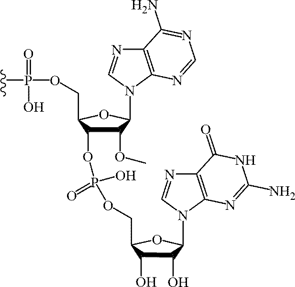

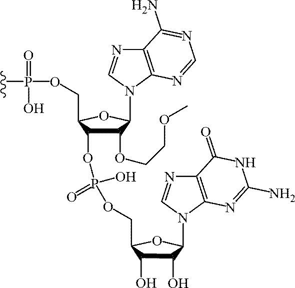

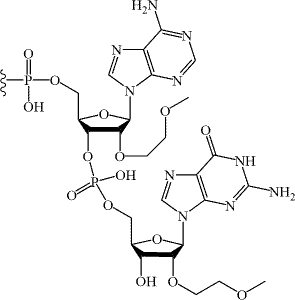

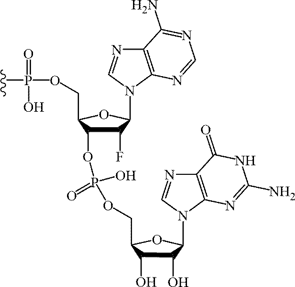

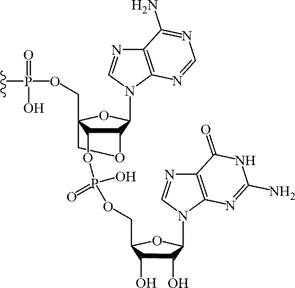

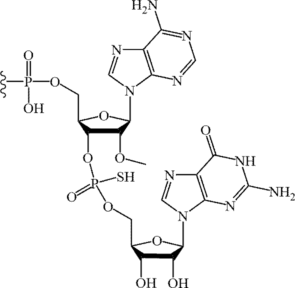

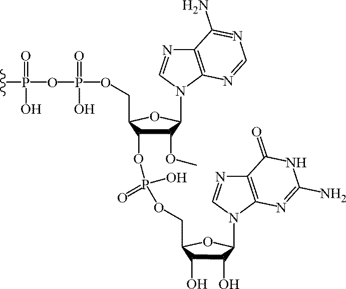

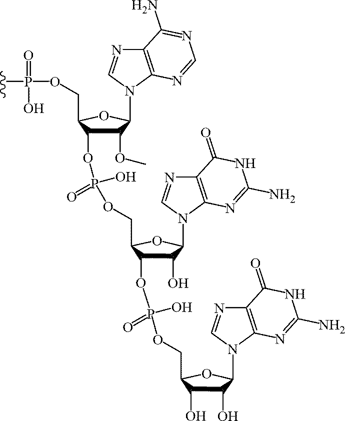

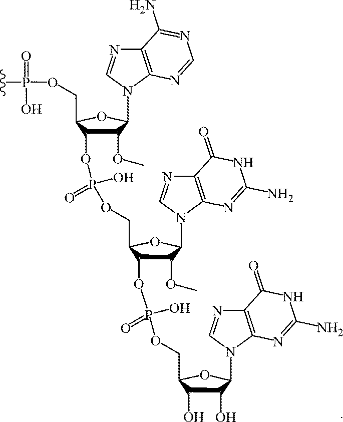

In another aspect, the present disclosure provides a compound of formula (Ib1), (Ib2), (Ib3) or (Ib4) below or a stereoisomer, tautomer or salt thereof:

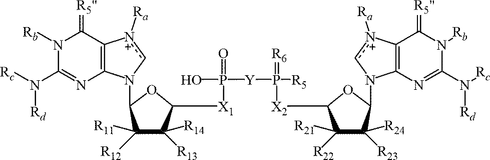

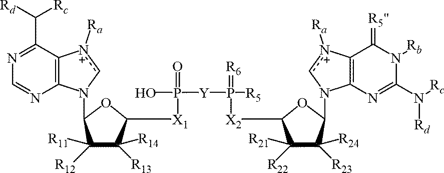

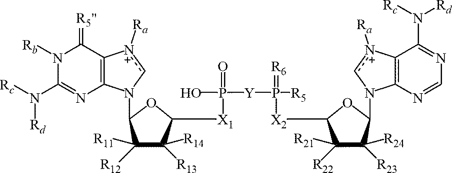

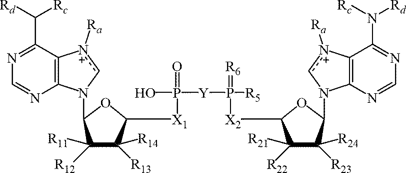

In certain embodiments, each Rₐ independently is H, C₁₋₆ alkyl, C₂₋₆ alkenyl, or C₂₋₆ alkynyl, each of which is optionally substituted with one or more substituents selected from the group consisting of halogen, C₁₋₆ alkoxyl, C₃₋₈ cycloalkyl, or C₃₋₈ heterocycloalkyl;
each R_{b} independently is H, NH₂, or C₁₋₆ alkyl;
each R_{c} independently is H, C₁₋₆ alkyl, or an amine protecting group;
each R_{d} independently is H, C₁₋₆ alkyl, or an amine protecting group;
each R₅″ independently is O or S.

For example, the “amine protecting group” includes but is not limited to fluorenylmethyloxycarbonyl (“Fmoc”), carboxybenzyl (“Cbz”), tert-butyloxycarbonyl (“BOC”), dimethoxybenzyl (“DMB”), acetyl (“Ac”), trifluoroacetyl, phthalimide, benzyl (“Bn”), Trityl (triphenylmethyl, Tr), benzylideneamine, or Tosyl (Ts).

In certain embodiments, X₁ and X₂ are independently selected from —(C(R₁)₂)ₘ—, —O—(C(R₁)₂)ₘ —, — (C(R₁)₂)ₘ—O—, or —CH═CH—; wherein each R₁ independently is H, F, Cl, Br, I or C₁₋₃ alkyl, wherein m is an integer selected from 0-6.

For example, each of X₁ and X₂ independently is —CH₂—O—, —O—CH₂—, —CH₂—CH₂—, —O—CH(CH₃)—, —CH(CH₃)—O—, or —CH═CH—.

In certain embodiments, Y is —(R₂—P(O)—R₂)ₚ—, wherein each R₂ independently is O, S, or C₁₋₆ alkyl, in which C₁₋₆ alkyl is optionally substituted with one or more of halogen or OH; and wherein p is 0, 1, or 2.

For example, Y is selected from

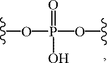

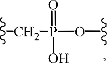

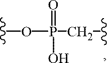

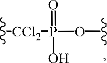

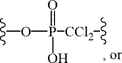

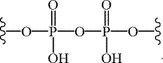

In certain embodiments, each of R₁₁, R₁₂, R₂₃ and R₂₄ independently is H, F, Cl, Br, I, or OR₃; each of R₁₃, R₁₄, R₂₁, and R₂₂ independently is H, F, Cl, Br, I, OR₃, or LNA; each R₃ independently is H, C₁₋₆ alkyl, C₂₋₆ alkenyl, or C₂₋₆ alkynyl, in which C₁₋₆ alkyl, C₂₋₆ alkenyl, or C₂₋₆ alkynyl is optionally substituted with one or more of F, Cl, Br, I, OH and C₁₋₆ alkoxyl;

In certain embodiments, each of R₁₁, R₁₂, R₂₃ and R₂₄ independently is H, F, Cl, Br, I or OR₃; R₁₃ and R₁₄ independently is H, F, Cl, Br, I, OR₃, or LNA; any one of R₂₁ and R₂₂ independently is H, F, Cl, Br, I, or OR₃, and the other is OR₄, wherein,
each R₃ independently is H, C₁₋₆ alkyl, C₂₋₆ alkenyl, or C₂₋₆ alkynyl, in which C₁₋₆ alkyl, C₂₋₆ alkenyl, or C₂₋₆ alkynyl is optionally substituted with one or more of F, Cl, Br, I, OH and C₁₋₆ alkoxyl;
R₄ independently is

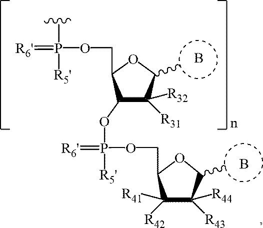

wherein,
each B are the same or different, and independently is a nucleobase or a modified nucleobase, wherein said nucleobase is selected from adenine (A), cytosine (C), guanine (G), thymine (T), or uracil (U), or said modified nucleobase is selected from hypoxanthine, xanthine, 7-methylguanine, 7-ethylguanine, 7-isopropylguanosine, 7-cyclopropylguanosine, 7-methyl-N1-methylguanosine, 7-(oxetanyl-methyl)guanosine, 7-(oxetanyl-ethyl)guanosine, 6-thioguanosine, 7-methyl-(6-thio)guanosine, 7-deaza-8-aza-guanosine, 7-methyl-7-deaza-8-aza-guanosine, 6-methyladenosine, 7-methyladenosine, 7-methylxanthine, 7-methyl hypoxanthine, 5,6-dihydrouracil, 5-methylcytosine, or dihydrouridine;
R₃₁, R₃₂, R₄₃, and R₄₄ are independently selected from H, F, Cl, Br, I, or OR₄′;
R₄₁ and R₄₂ are independently selected from H, F, Cl, Br, I, OR₄′, or LNA;
each R₄′ is independently selected from H, C₁₋₆ alkyl, C₂₋₆ alkenyl, or C₂₋₆ alkynyl, in which C₁₋₆ alkyl, C₂₋₆ alkenyl, or C₂₋₆ alkynyl is optionally substituted with one or more of F, Cl, Br, I, OH and C₁₋₆ alkoxyl;
each R₅′ independently is OH or SH;
each R₆′ independently is O or S;
n is 0, 1, or 2.

In certain embodiments, R₄ independently is selected from the group consisting of:

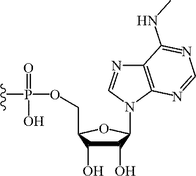

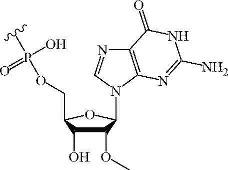

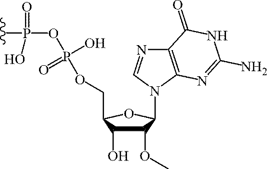

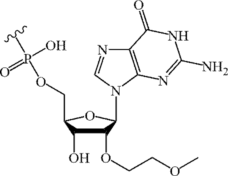

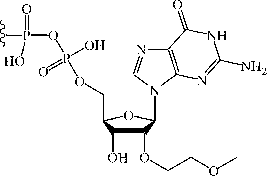

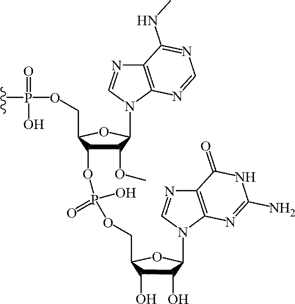

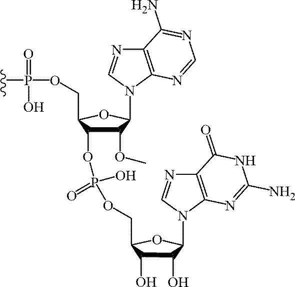

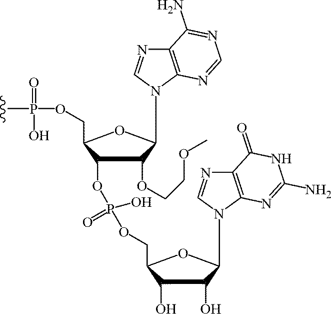

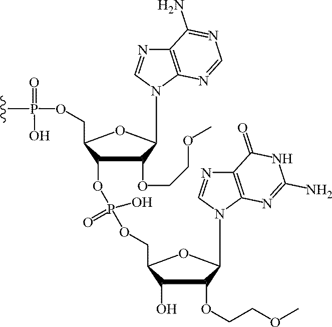

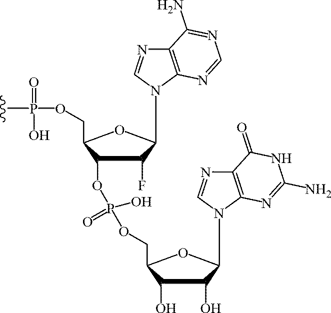

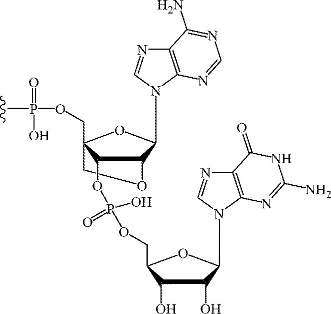

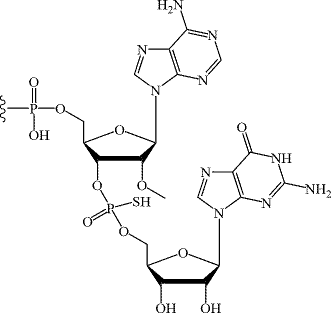

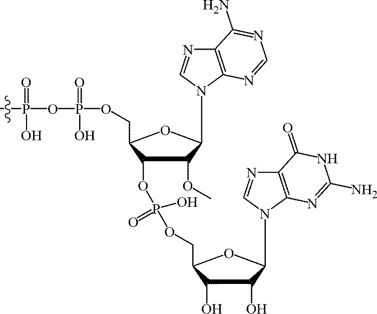

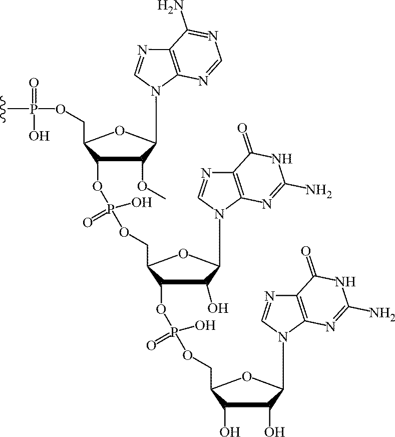

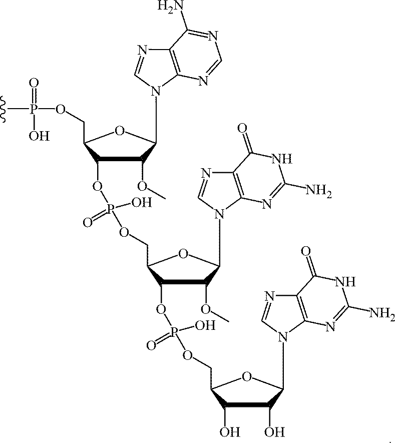

In another aspect, the present disclosure provides a compound of formula (II) below or a stereoisomer, tautomer, deuterate or salt thereof:

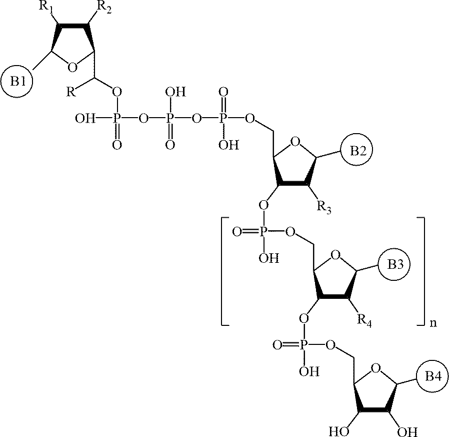

wherein:
ring B1, B2, B3 and B4 are each independently selected from a nucleobase and a modified nucleobase;
each R is independently selected from H and C₁₋₆ alkyl;
R₁ and R₂ are each independently selected from OR₅ and halogen;
each R₃ is independently selected from C₁₋₆ alkoxyl, halogen and LNA;
each R₄ is independently selected from halogen and LNA;
each R₅ is independently selected from H, C₁₋₆ alkyl, C₂₋₆ alkenyl and C₂₋₆ alkynyl, in which C₁₋₆ alkyl, C₂₋₆ alkenyl and C₂₋₆ alkynyl is optionally substituted with one or more of halogen, OH, and/or C₁₋₆ alkoxyl; and
n is an integer selected from 0-1.

In certain embodiments, ring B1 and B4 are each independently guanine (G), and ring B2 and B3 are each independently adenine (A).

In certain embodiments, R is independently selected from H and methyl.

In certain embodiments, R₁ and R₂ are each independently selected from OR₅, F, Cl, Br and I, and each R₅ is independently selected from H and C₁₋₆ alkyl, in which C₁₋₆ alkyl is optionally substituted with C₁₋₆ alkoxyl.

In certain embodiments, R₁ and R₂ are each independently selected from OH, —O(CH₂)₂OCH₃ and F.

In certain embodiments, R₃ is independently selected from methoxy, F and LNA.

In certain embodiments, R₄ is independently selected from F and LNA.

In another aspect, the present disclosure provides a compound of formula (IIA) below or a stereoisomer, tautomer, deuterate or salt thereof:

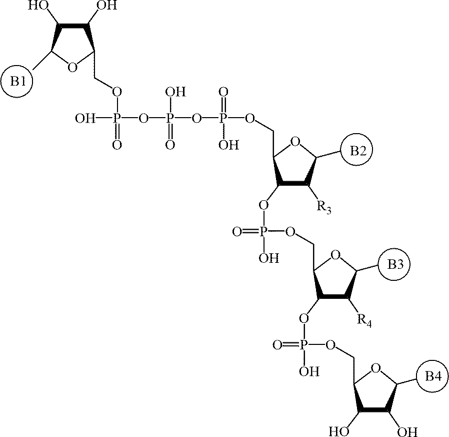

wherein:
ring B1, B2, B3 and B4 are each independently selected from a nucleobase and a modified nucleobase; and
R₃ and R₄ are each independently selected from halogen and LNA.

In certain embodiments, ring B1 and B4 are each independently guanine (G), and ring B2 and B3 are each independently adenine (A).

In certain embodiments, R₃ and R₄ are each independently selected from F and LNA.

In another aspect, the present disclosure provides a compound of formula (IIB) below or a stereoisomer, tautomer, deuterate or salt thereof:

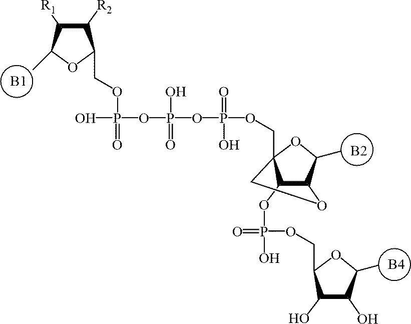

wherein:
ring B1, B2 and B4 are each independently selected from a nucleobase and a modified nucleobase;
R₁ and R₂ are each independently selected from OR₅ and halogen; and
each R₅ is independently selected from H, C₁₋₆ alkyl, C₂₋₆ alkenyl and C₂₋₆ alkynyl, in which C₁₋₆ alkyl, C₂₋₆ alkenyl and C₂₋₆ alkynyl is optionally substituted with one or more of halogen, OH, and/or C₁₋₆ alkoxyl.

In certain embodiments, ring B1 and B4 are each independently guanine (G), and ring B2 is adenine (A).

In certain embodiments, R₁ and R₂ are each independently selected from OR₅, F, Cl, Br and I, and each R₅ is independently selected from H and C₁₋₆ alkyl, in which C₁₋₆ alkyl is optionally substituted with C₁₋₆ alkoxyl.

In certain embodiments, R₁ and R₂ are each independently selected from OH, —O(CH₂)₂OCH₃ and F.

In another aspect, the present disclosure provides a compound, stereoisomer, tautomer, deuterate or salt selected from any of:

| No. | Chemical name | Chemical structure |
|---|---|---|
| GL-Cap 1 | 7mGppp(locked)Ap(locked)ApG | 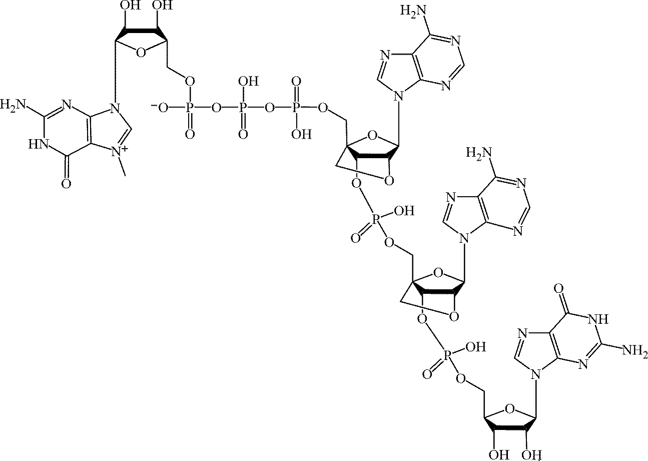 |
| GL-Cap 2 | 7mGppp(2-′F)Ap(2′-F)ApG | 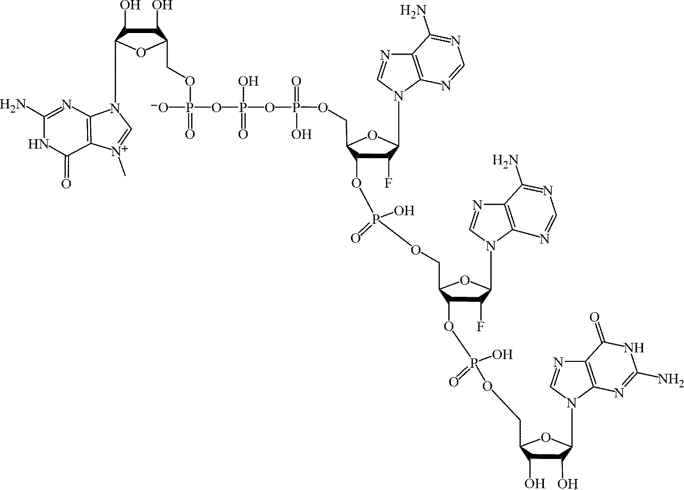 |
| GL-Cap 3 | 7mGppp(2′-F)Ap(locked)ApG | 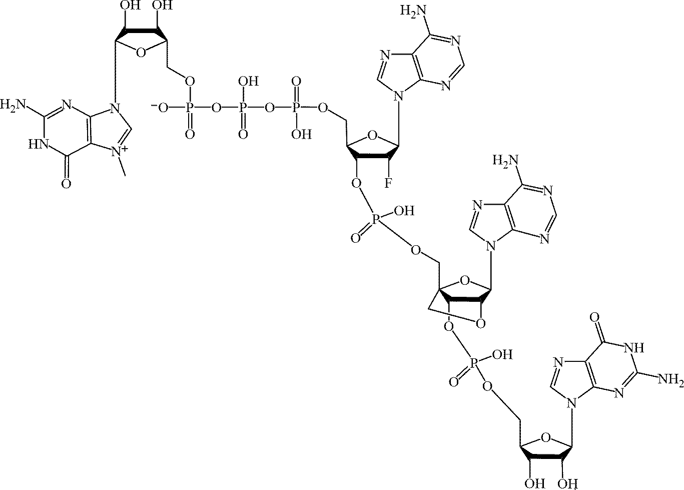 |
| GL-Cap 4 | 7mGppp(locked)Ap(2′-F)ApG | 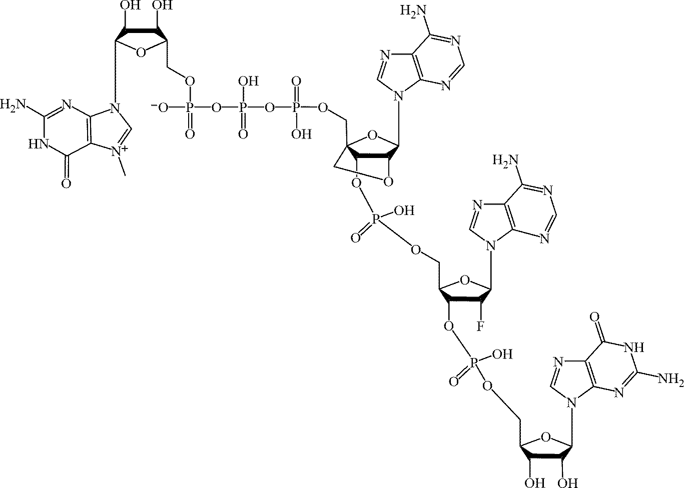 |
| GL-Cap 5 | 7mGppp(locked)ApG | 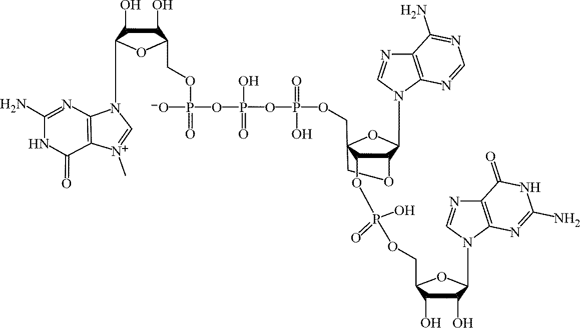 |
| GL-Cap 6 | 7m-3′-MOEGppp(locked)ApG | 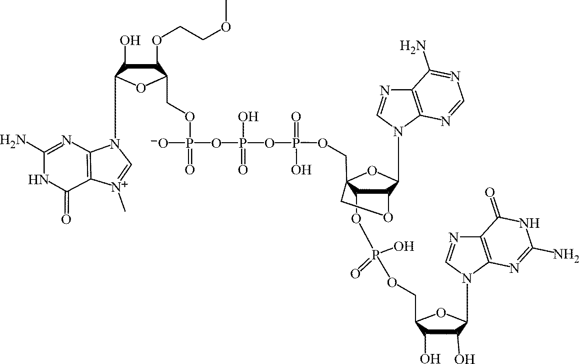 |
| GL-Cap 7 | 7m-2′-FGppp(locked)ApG | 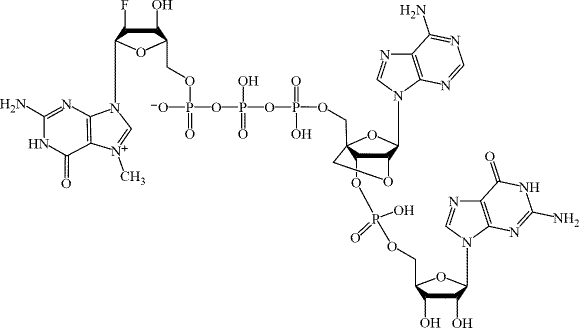 |
| GL-Cap 8 | 7m-2′ -MOEGppp(locked)ApG | 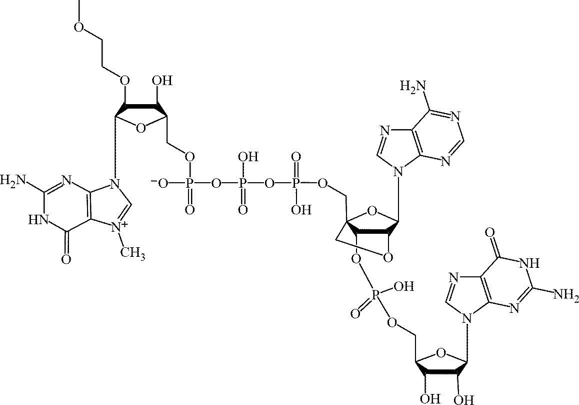 |
| GL-Cap 9 | 7m-3′-MOE-5′-RmGppp(2′-Ome)ApG | 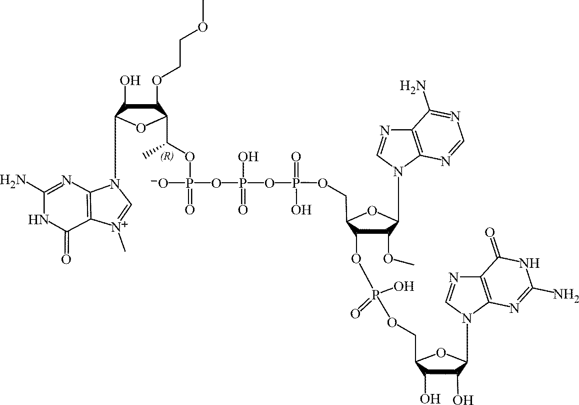 |

Unless otherwise defined, all technical and scientific terms used herein have the same meaning as commonly understood by one of ordinary skill in the art to which this invention belongs. In the specification, the singular forms also include the plural unless the context clearly dictates otherwise. Although methods and materials similar or equivalent to those described herein can be used in the practice or testing of the present invention, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

### Definition

For the purpose of the current disclosure, the following definitions shall in their entireties be used to define technical terms, and shall also, in their entireties, be used to define the scope of the composition of matter for which protection is sought in the claims.

As used herein, the term “LNA” or “locked nucleic acid” refers to a methylene bridge between the 2′O and 4′C of the nucleotide monomer or to a sugar analog, a nucleoside, a nucleotide monomer, or a nucleic acid, each of which contains such bridge. For example,

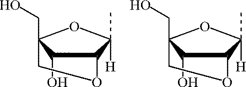

or those described in WO99/14226 and Kore et al, J. AM. CHEM. SOC. 2009, 131, 6364-6365, the contents of each of which are incorporated herein by reference in their entireties.

As used herein, the term “nucleobase” refers to a nitrogen-containing heterocyclic moiety, which are the parts of the nucleic acids that are involved in the hydrogen-bonding that binds one nucleic acid strand to another complementary strand in a sequence-specific manner. The most common naturally-occurring nucleobases are : adenine (A), cytosine (C), guanine (G), thymine (T), and uracil (U).

The term “modified nucleobase” refers to a moiety that can replace a nucleobase. The modified nucleobase mimics the spatial arrangement, electronic properties, or some other physicochemical property of the nucleobase and retains the property of hydrogen-bonding that binds one nucleic acid strand to another in a sequence-specific manner. A modified nucleobase can pair with at least one of the five naturally occurring bases (uracil, thymine, adenine, cytosine, or guanine) without substantially affecting the melting behavior, recognition by intracellular enzymes, or activity of the oligonucleotide duplex. The term “modified nucleoside” or “modified nucleotide” refers to a nucleoside or nucleotide that contains a modified nucleobase and/or other chemical modification disclosed herein, such as modified sugar, modified phosphorus atom bridges or modified internucleoside linkage.

Non-limiting examples of suitable nucleobases include, but are not limited to, hypoxanthine, xanthine, 7-methylguanine, 7-ethylguanine, 7-isopropylguanosine, 7-cyclopropylguanosine, 7-methyl-N1-methylguanosine, 7-(oxetanyl-methyl)guanosine, 7-(oxetanyl-ethyl)guanosine, 6-thioguanosine, 7-methyl-(6-thio)guanosine, 7-deaza-8-aza-guanosine, 7-methyl-7-deaza-8-aza-guanosine, 6-methyladenosine, 7-methyladenosine, 7-methylxanthine, 7-methyl hypoxanthine, 5,6-dihydrouracil, 5-methylcytosine, and dihydrouridine.

As used herein, “halo” or “halogen” refers to fluoro, chloro, bromo and iodo.

The term “alkyl” and “C₁₋₆-alkyl” are intended to mean a linear or branched saturated hydrocarbon chain wherein the longest chain has from one to six carbon atoms, such as methyl, ethyl, n-propyl, i-propyl, n-butyl, s-butyl, t-butyl, n-pentyl, s-pentyl and n- hexyl.

The term “alkenyl” and “C₁₋₆ alkenyl” are intended to mean a linear or branched unsaturated hydrocarbon chain wherein the longest chain has from two to six carbon atoms, such as ethenyl, propenyl, butenyl, pentenyl, hexenyl, and branched alkenyl groups.

The term “alkynyl” and “C₁₋₆ alkynyl” are intended to mean a linear or branched unsaturated hydrocarbon chain, which contain at least one triple bond, wherein the longest chain has from two to six carbon atoms, such as ethynyl, propynyl, butynyl, pentynyl, hexynyl, and branched alkynyl groups.

The term “alkoxy” and “C₁₋₆ alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy, pentoxy and hexyloxy groups.

As used herein, “carbocycle”,“cycloalkyl” and “carbocyclic ring” is intended to include any stable monocyclic or bicyclic ring having the specified number of carbons, any of which may be saturated or unsaturated. For example, C₃₋₈ cycloalkyl is intended to include a monocyclic or bicyclic ring having 3, 4, 5, 6, 7 or 8 carbon atoms. Examples of carbocycles include, but are not limited to, cyclopropyl, cyclobutyl, cyclobutenyl, cyclopentyl, cyclopentenyl, cyclohexyl.

As used herein, “heterocycle”, “heterocycloalkyl” or “heterocyclic group” includes any ring structure (saturated or unsaturated) which contains at least one ring heteroatom (e.g., N, O or S). Heterocycle includes heterocycloalkyl. Examples of heterocycloalkyl include, but are not limited to, morpholine, pyrrolidine, tetrahydrothiophene, piperidine, piperazine, oxetane, pyran, tetrahydropyran, azetidine, and tetrahydrofuran.

The term “amine protecting group” refers to a protecting group for amines. Examples of amine protecting groups include but are not limited to fluorenylmethyloxycarbonyl (“Fmoc”), carboxybenzyl (“Cbz”), tert-butyloxycarbonyl (“BOC”), dimethoxybenzyl (“DMB”), acetyl (“Ac”), trifluoroacetyl, phthalimide, benzyl (“Bn”), Trityl (triphenylmethyl, Tr), benzylideneamine, Tosyl (Ts). See also Chem. Rev. 2009, 109, 2455-2504 for additional amine protecting groups, the contents of which are incorporated herein by reference in their entirety.

The term “stereoisomer” refers to a compound which is composed of the same atom, bonded by the same bond, but has a different three-dimensional structure. The present invention will cover various stereoisomers and mixtures thereof.

The term “tautomer” refers to the isomer formed by transferring a proton from one atom of a molecule to another atom of the same molecule. All tautomeric forms of the compounds of the present invention will also be included within the scope of the present invention.

The compounds of the present invention may contain one or more chiral carbon atoms, and thus may produce an enantiomer, diastereomer and other stereoisomeric forms. Each chiral carbon atom may be defined as (R)- or (S)- based on stereochemistry. The compounds of the invention include all possible isomers, and their racemates and optically pure forms. The preparation of the compounds of the present invention can be selected as a racemate, diastereomer or enantiomer as a raw material or an intermediate. The optically active isomers may be prepared using chiral synthons or chiral reagents, or by conventional techniques, for example using methods such as crystallization and chiral chromatography.

The term “deuterate” refers to a deuterium-containing compound that is generated by replacement of one or more available hydrogen atoms in the compound with a corresponding number of deuterium atoms. For example, the -CH₃ on the guanine (G) of the present compound could be deuterated as -CD₃, i.e.,

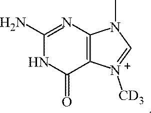

The compounds of the present invention may be deuterated in a manner known to those of ordinary skill in the art, including in the methods of the present invention described below.

### Head Nucleoside Group Modifications

Head group modifications, including nucleoside, sugar and 7-alkyl modifications, are summarized in Table 1. We made sugar modifications of 7mG by incorporating 2′- or 3′-methoxyethyl group (MOE). A methoxy ethyl modification on 2′-ribose is well-documented in oligonucleotides for antisense applications to increase oligonucleotide stability against nucleases and hybridization affinity to the target. However, its utility in mRNA 5′-cap structures has not been explored. Similarly, the utility of other substitutions such as 2′-fluoro are not known in the 5′-cap structures. In addition, in some embodiments, we have also completely replaced ribose sugar with arabinose or 2′-3′-substituted arabinose sugar in the 5′-cap head group. Additionally, enantiomerically pure L-guanosine (L-G) analogs have also been utilized in place of D-guanosine. Representative compounds with these modifications have the following structure:

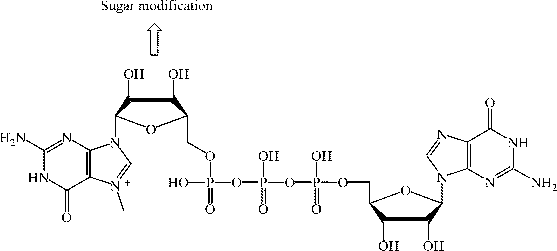

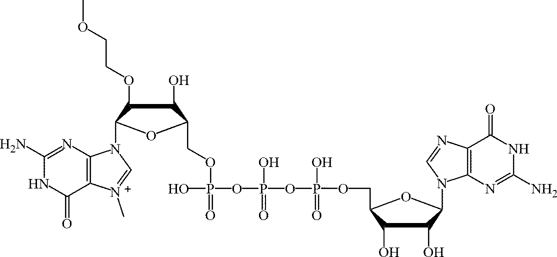

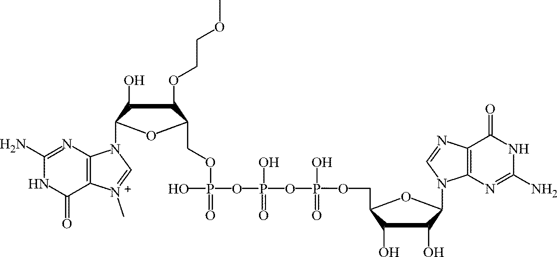

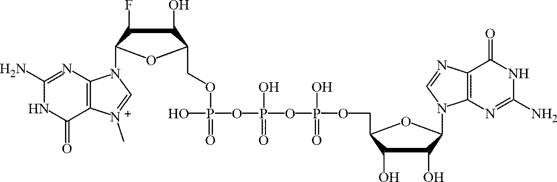

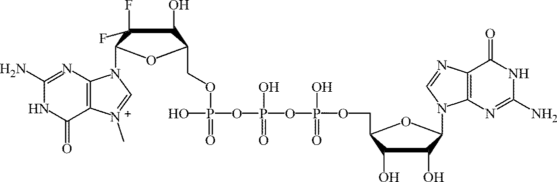

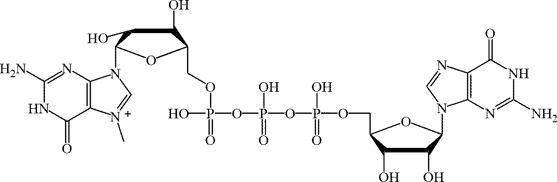

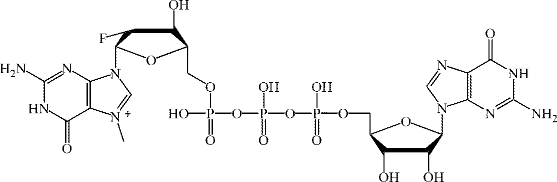

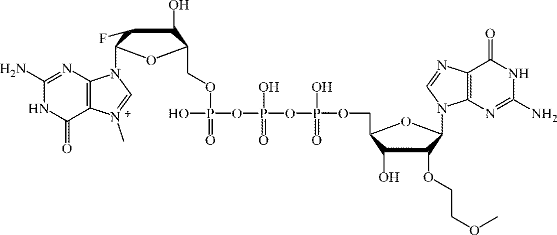

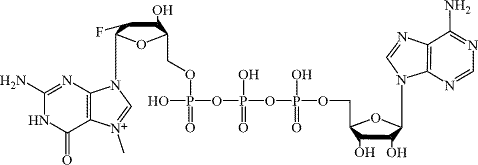

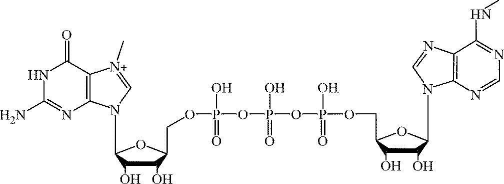

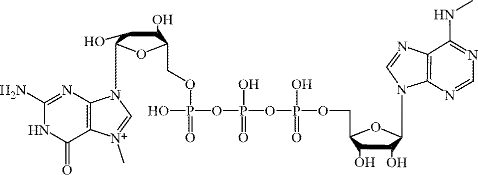

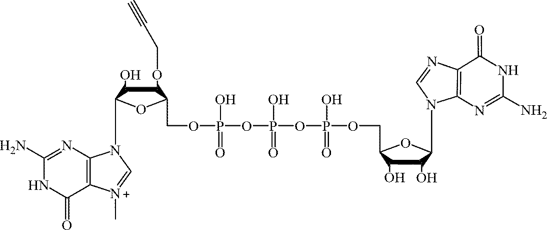

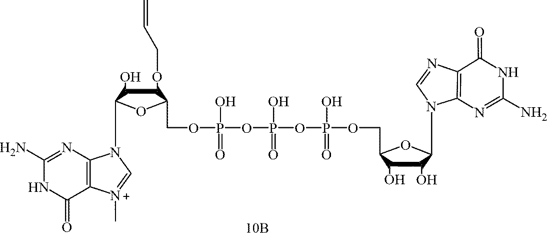

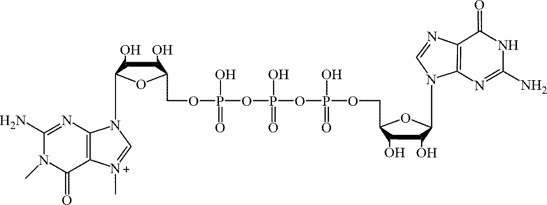

TABLE 1

5′-Cap analogs resulting from head nucleoside group modification.

| Compound # | Structure | MS (Calc) | MS (found) |
|---|---|---|---|
| 1 | 7m(2′-moe)GpppG | 861.1 | 861.0 |
| 2 | 7m(3′-moe)GpppG | 861.1 | 861.0 |
| 3 | 7m(2′-F)GpppG | 805.1 | -- |
| 4 | 7m(2′,2′-F₂)GpppG | 823.1 | -- |
| 5 | 7m(ara-G)pppG | 803.1 | -- |
| 6 | 7m(2′-F-araG)pppG | 806.1 | 806.0 |
| 7 | 7m(2′-F-araG)ppp(2′-moeG) | 863.1 | 863.0 |
| 8 | 7m(2′-F-araG)pppA | 789.1 | -- |
| 9 | 7m(L-G)ppp(6mA) | 801.1 | -- |
| 10 | 7m(ara-G)ppp(6mA) | 801.1 | -- |
| 10A | 7m(3′-propargyl-G)pppG | 841.5 | -- |
| 10B | 7m(3′-allyl-G)pppG | 843.5 | -- |
| 10C | 7m(N1-methyl-G)pppG | 817.5 | -- |
| 11 | 7-e-GpppG | 817.1 | 817.2 |
| 12 | 7-iPr-GpppG | 831.1 | 832.0 |
| 13 | 7-(2-F-ethyl)-GpppG | 835.1 | 835.2 |
| 14 | 7-(cPr-methyl)-GpppG | 843.1 | 843.2 |
| 15 | 7-(oxetanyl-methyl)-GpppG | 859.1 | -- |
| 16 | 7-(oxetanyl-ethyl)-GpppG | 873.1 | -- |
| 17 | 7mIpppG | 788.1 | 786.6 |
| 18 | 7mIppp(2′moe-A) | 830.1 | 828.4 |
| 19 | 7mIppp(6-thio-G) | 804.1 | 804.4 |
| 19A | 7mGppp(6-thio-G) | 819.2 | |
| 20 | 7mXpppG | 804.1 | 804.4 |
| 21 | 7m(6-thio-G)ppp(6mA) | 817.1 | -- |
| 22 | 7m(8-aza-G)pppG | 802.1 | -- |
| 23 | 7m3′m(8-aza-G)pppG | 816.1 | -- |
| 24 | 7m(3′-moe-5′-_{R}mG)pppG | 875.2 | -- |
| 25 | 7m(2′-moe-5′-_{R}mA)pppG | 859.2 | -- |

In our nucleoside modifications, 7mG nucleoside head group was modified into various 7-alkylated guanosine groups. The rational was to increase the size and add an additional hetero-atom at the terminal of 7-alkyl to improve additional interactions between the 5′-cap analog and its receptors such as eIF4E (eukaryotic translation initiation factor 4E). Representative compounds with various 7-alkylated guanosine groups have the following structure:

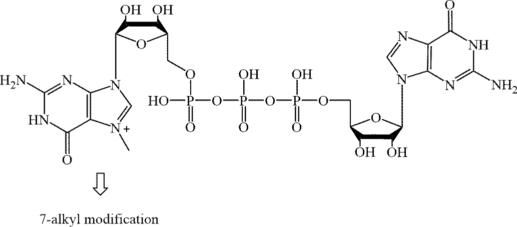

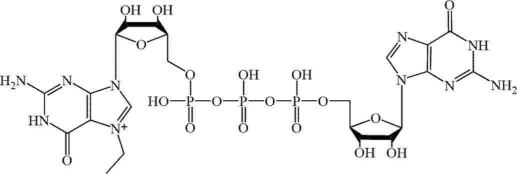

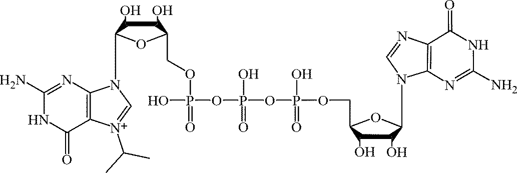

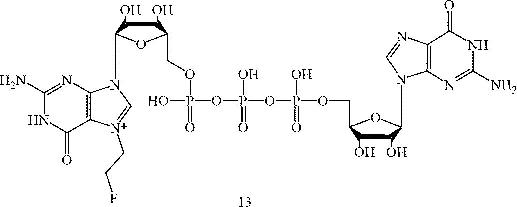

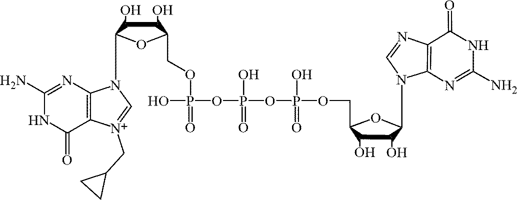

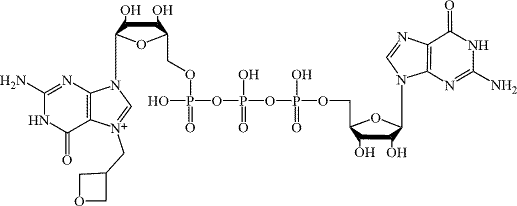

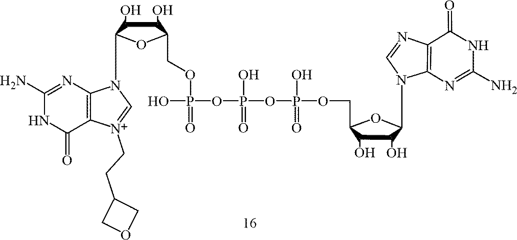

Nucleobase analogs of guanosine were adopted for modifications, including inosine (I), xanthosine (X), and 6-thioguanosine (6-thio-G) analogs. 7-deaza-8-aza-guanosine (8-aza-G) analogs are of particular interest as a neutral alternative to 7mG. 5′-(R)-methyl modified nucleosides (5′-_{R}mN; N=G or A) can be incorporated similarly. Representative compounds with nucleobase analogs of guanosine have the following structure:

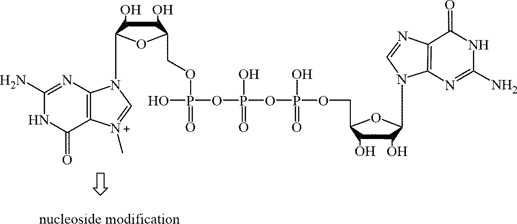

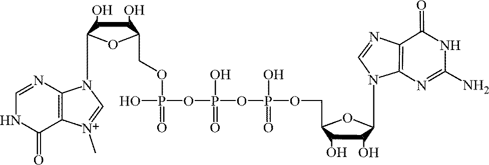

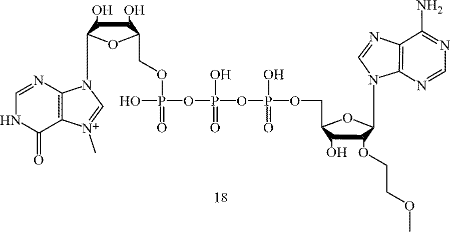

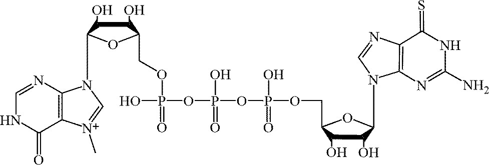

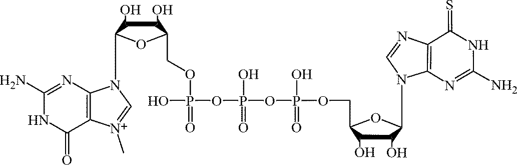

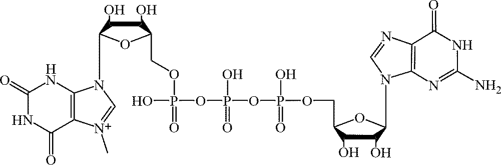

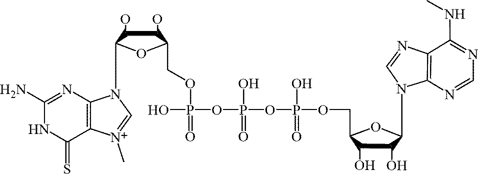

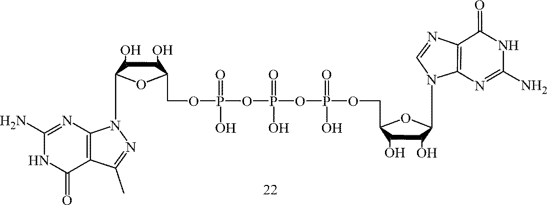

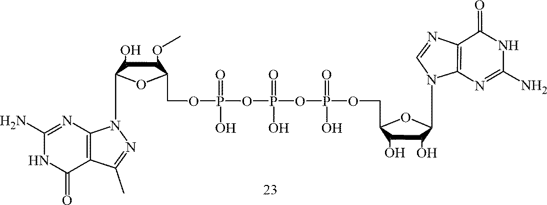

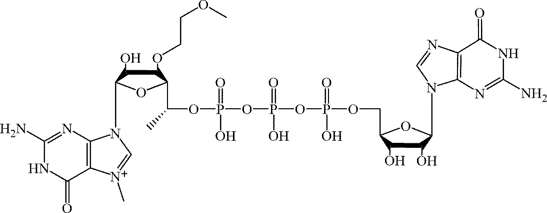

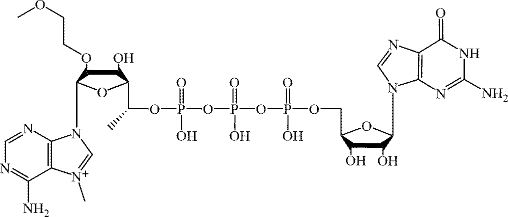

### Triphosphate Linker Modifications

γ-phosphate on the 7 mG head group was modified to a phosphonate and used as a building block for modified cap analogs. 5′-Vinyl phosphonate and 5′-ethyl phosphonates of guanosine were synthesized and incorporated into cap structures either as a head nucleoside group or as a tail ribonucleoside group analog via a magnesium-mediated ligation reaction. The same modifications were adopted as α-phosphate modifications. The replacement of an inter-phosphate oxygen atom by a methylene group or a dichloro-methylene group generated triphosphate analogs. Tetraphosphates are also possible to link optimal head and tail groups. Representative compounds with triphosphate linker modifications have the following structures (linker modifications are summarized in Table 2):

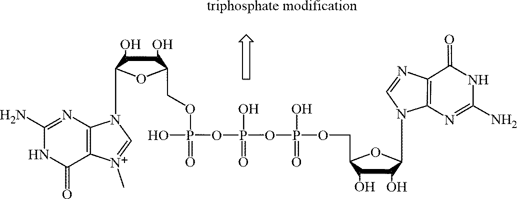

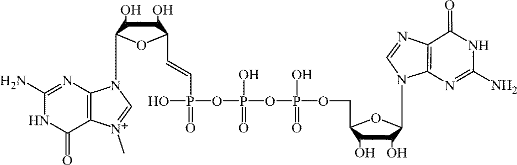

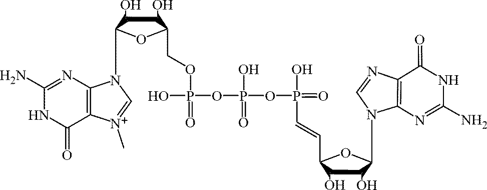

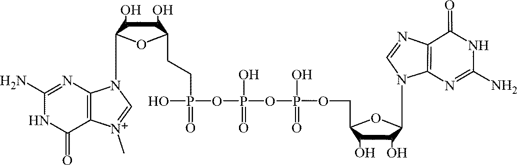

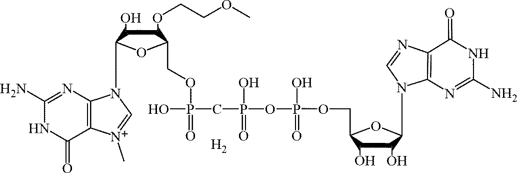

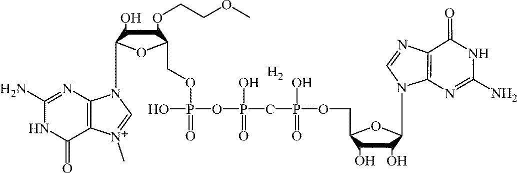

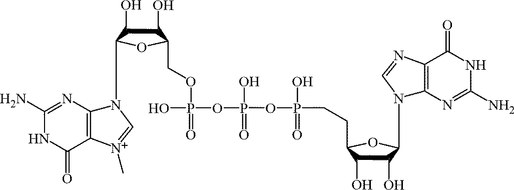

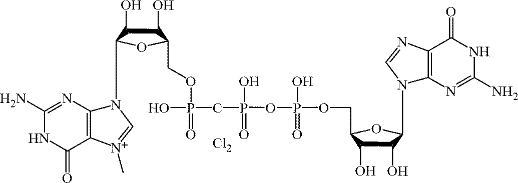

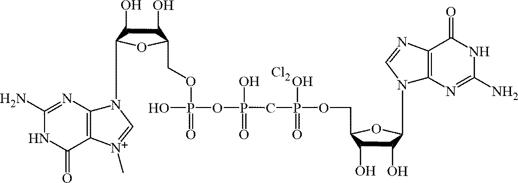

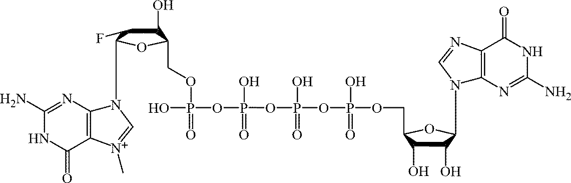

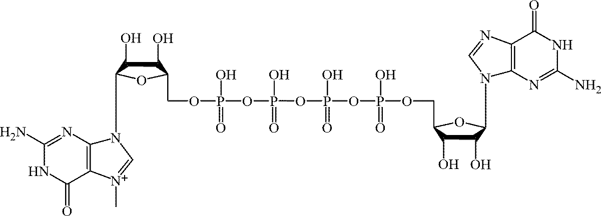

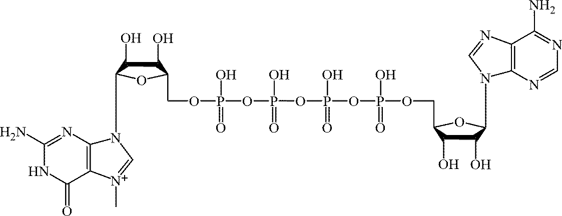

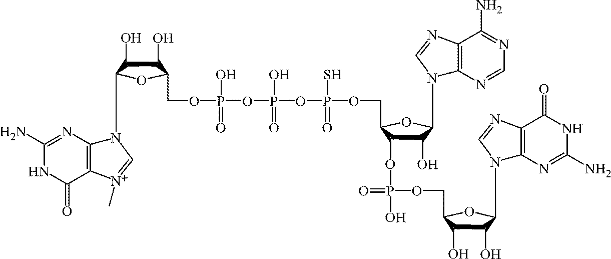

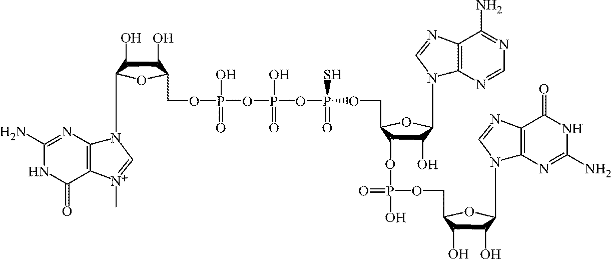

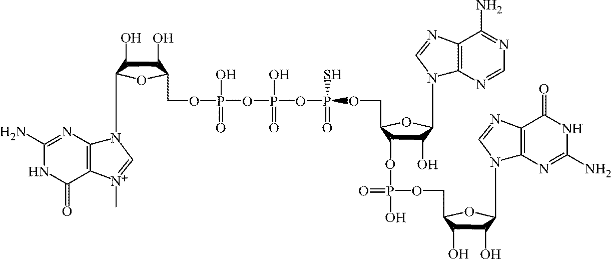

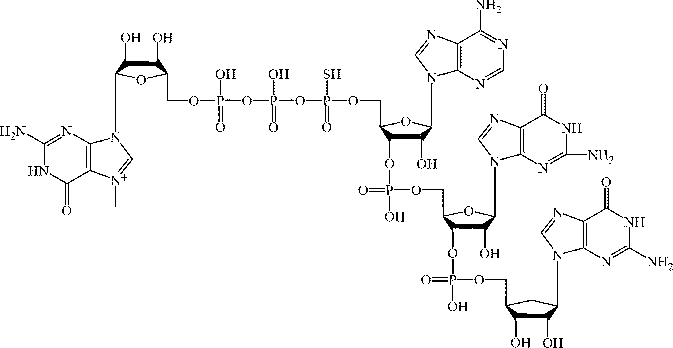

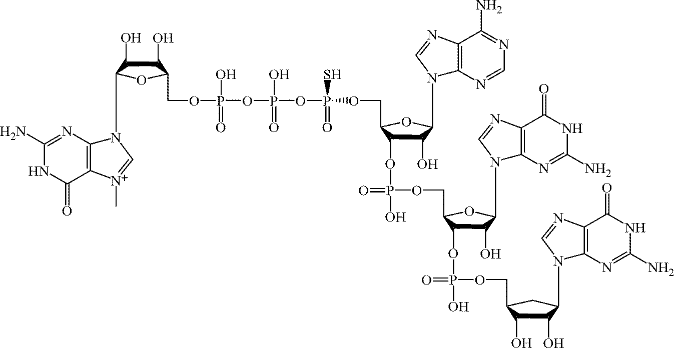

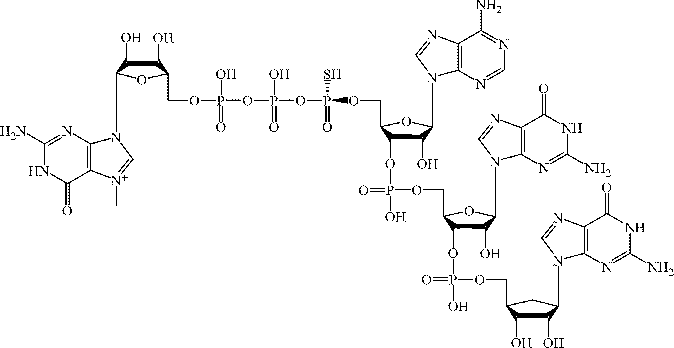

TABLE 2

5′-Cap analogs with modifications in the triphosphate linker.

| Compound # | Sequence | MS (Calc‘ed) | MS (found) |
|---|---|---|---|
| 26 | 7-(5′vinyl-G)pppG | 799.1 | 799.0 |
| 27 | 7mGppp(5′vinyl-G) | 799.1 | 799.0 |
| 28 | 7mG_{CH2}pppG | 801.1 | -- |
| 29 | 7m(3′ -moe)Gp_{CH2}ppG | 859.2 | -- |
| 30 | 7m(3′ -moe)Gpp_{cH2}pG | 859.2 | -- |
| 31 | 7mGppp_{CH2}G | 801.1 | 801.4 |
| 32 | 7mGp_{CCl2}ppG | 869.0 | -- |
| 33 | 7mGpp_{CCl2}pG | 869.0 | -- |
| 34 | 7m(2′-F-araG)ppppG | 885.1 | -- |
| 34A | 7mGppp(s)G | 899.5 | -- |
| 34B | 7mGppp(s)A | 883.5 | -- |
| 34C | 7mGppp(s)ApG | 1148.7 | -- |
| 34D | 7mGppp(s-Sp)ApG | 1148.7 | -- |
| 34E | 7mGppp(s-Rp)ApG | 1148.7 | -- |
| 34F | 7mGppp(s)ApGpG | 1493.9 | -- |
| 34G | 7mGppp(s-Sp)ApGpG | 1493.9 | -- |
| 34H | 7mGppp(s-Rp)ApGpG | 1493.9 | -- |

### Tail Nucleoside Group Modifications

To introduce modifications in the tail nucleoside group of the 5′-cap structure, we utilized DNA/RNA synthesizer to produce 5′-phosphorylated mono-, di- and trinucleotides for ligation with selected head nucleoside groups that were synthesized. The pyro-phosphorylation of mono-, di- and trinucleotides were also made through solid phase oligo synthesis with slightly modified standard protocols, and the nucleosides synthesized are as follows (summarized in Table 5):

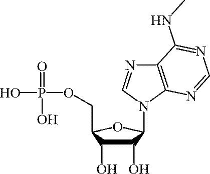

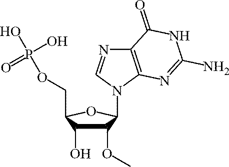

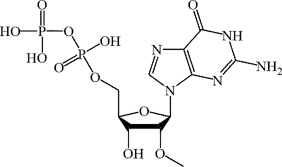

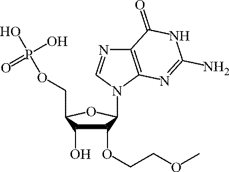

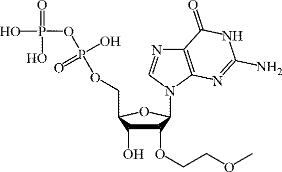

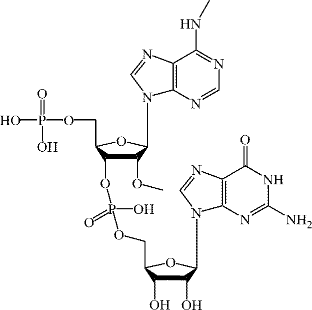

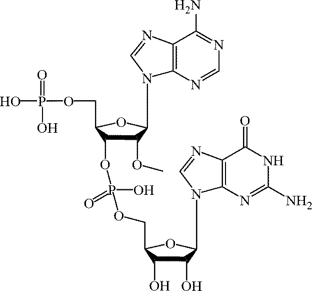

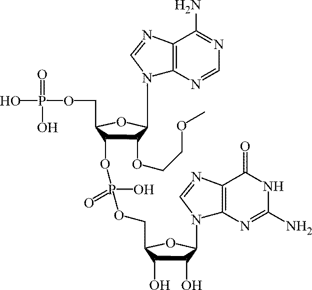

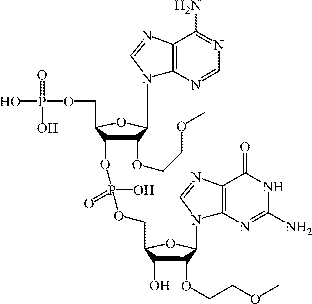

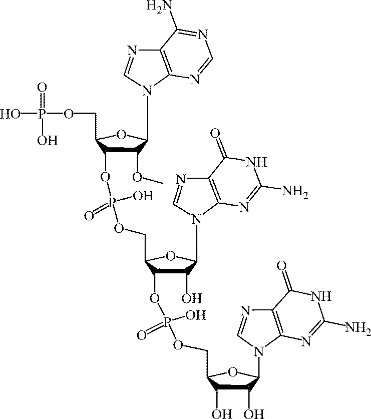

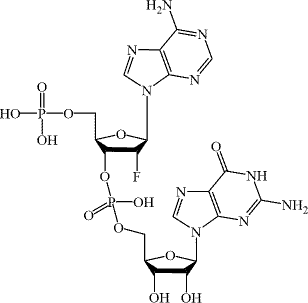

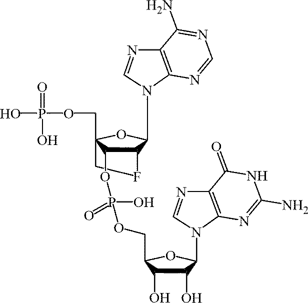

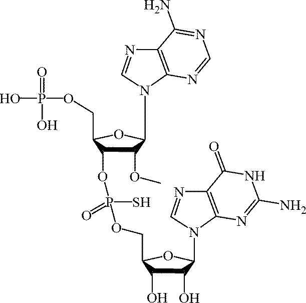

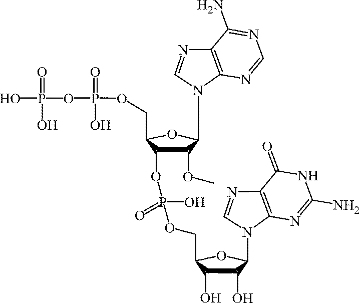

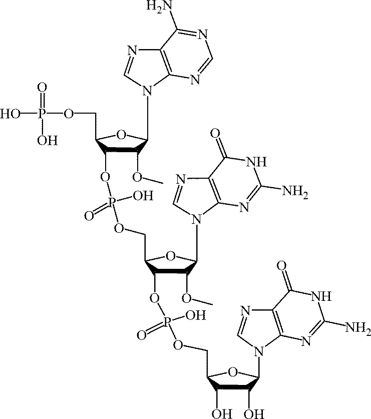

TABLE 3

5′-phosphorylated oligo building blocks to incorporate as the tail nucleoside groups; and the examples of ligated 5′-cap structures

| Compound # | Sequence | MS (Calc‘ed) | MS (found) |
|---|---|---|---|
| 35 | 5′-p(6mA) | 361.1 | -- |
| 36 | 5′-p(2′-omeG) | 377.1 | 378.0 |
| 37 | 5′-pp(2′-omeG) | 457.0 | 458.0 |
| 38 | 5′-p(2′-moeG) | 421.1 | -- |
| 39 | 5′-pp(2′-moeG) | 501.1 | -- |
| 40 | 5′-p(2′-ome-6mA)rG | 720.1 | -- |
| 41 | 5′-p(2′-omeA)rG | 706.1 | 705.0 |
| 42 | 5′-p(2′-moeA)rG | 750.2 | 749.0 |
| 43 | 5′-p(2′-moeA)(2′moeG) | 808.2 | 807.0 |
| 44 | 5′-p(2′-omeA)rGrG | 1051.2 | -- |
| 45 | 5′-p(2′-F-A)rG | 694.1 | -- |
| 46 | 5′-p(lockedA)rG | 704.1 | -- |
| 47 | 5′-p(2′-omeA)*rG | 722.1 | -- |
| 48 | 5′-pp(2′-omeA)rG | 786.1 | -- |
| 49 | 5′-p(2′-omeA)r(2′-omeG)rG | 1065.2 | -- |
| 50 | 7mGppp(6mA) | 801.1 | 801.0 |
| 51 | 7mGppp(6-thio-G) | 819.1 | 819.3 |
| 52 | 7mGppp(2′-moeG) | 861.1 | 861.0 |
| 53 | 7mGppp(2′-moeA) | 845.1 | 845.0 |
| 54 | 7m(5′vG)ppp(2′-omeA)rG | 1142.2 | -- |
| 55 | 7mGppp(2′-moeA)rG | 1190.2 | 1190.0 |
| 56 | 7mGppp(2′-moeA)(2′moeG) | 1248.2 | 625.0 |
| 57 | 7mGppp(2′-F-A)rG | 1134.1 | -- |
| 58 | 7m(3′-moeG)ppp(2′-omeA)rG | 1204.2 | -- |
| 59 | 7mGppp(2′-omeA)rGrG | 1491.2 | -- |
| 60 | 7mGppp(lockedA)rG | 1144.1 | -- |
| 61 | 7mGppp(2′-omeA)*rG | 1162.1 | -- |
| 62 | 7m(2′-F-ara-G)ppp(2′-omeA)rG | 1144.2 | -- |
| 62A | 7mGppp(2′-propargyl-G) | 841.5 | -- |
| 62B | 7mGppp(2′-allyl-G) | 843.5 | -- |
| 62C | 7mGppp(8-Chloro-G) | 837.9 | -- |
| 62D | 7mGppp(8-thio-G) | 835.5 | -- |
| 62E | 7mGppp(N1-methyl-G) | 817.5 | -- |
| 62F | 7mGppp(8-oxo-G) | 819.4 | -- |

With a proper 5′-phosphate purified as a triethylamine (TEA) salt, the ligation reaction to a suitable imidazole-activated head group provided 5′-5′ triphosphate linked polynucleotide cap analogs. Representative compounds with tail nucleoside group modifications have the following structure:

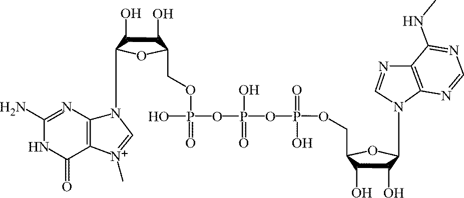

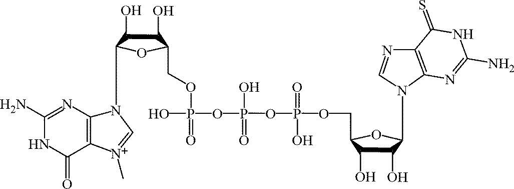

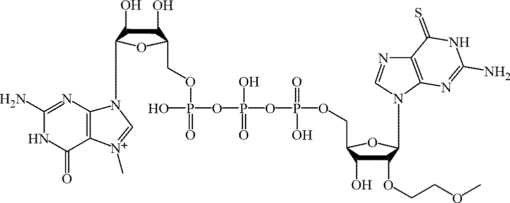

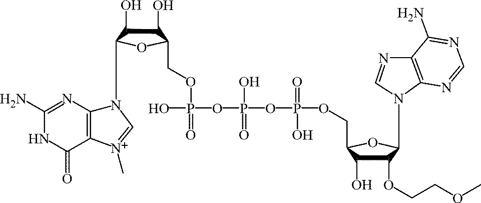

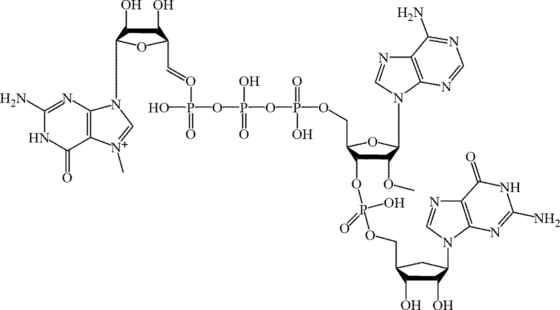

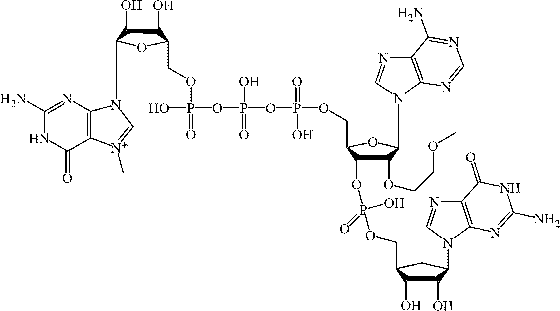

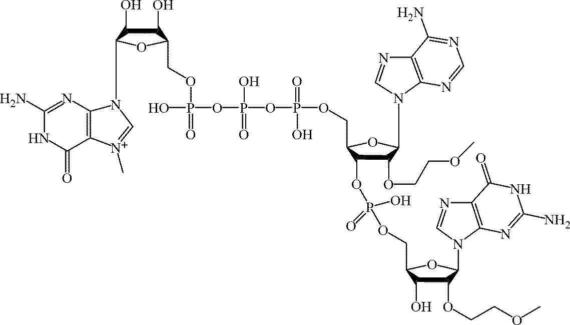

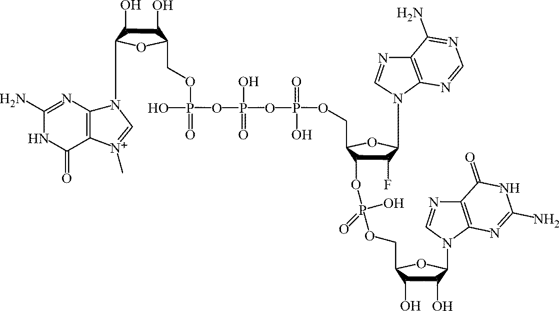

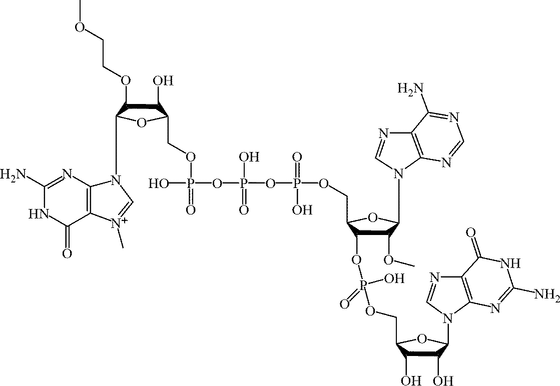

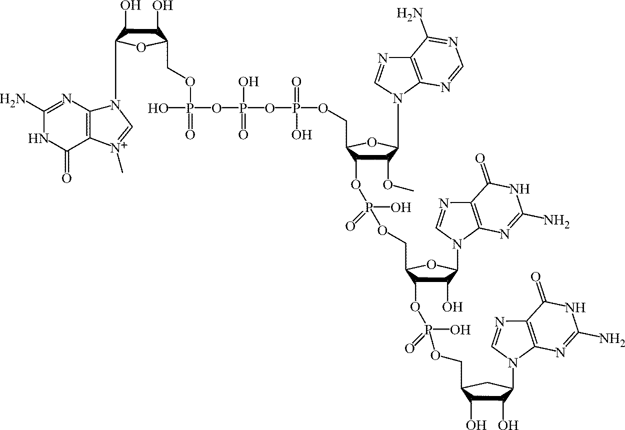

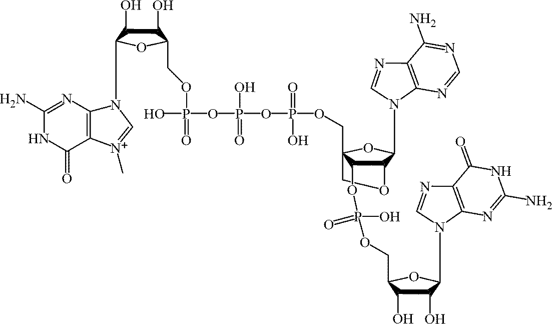

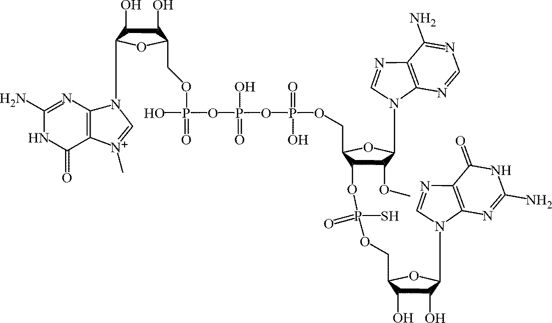

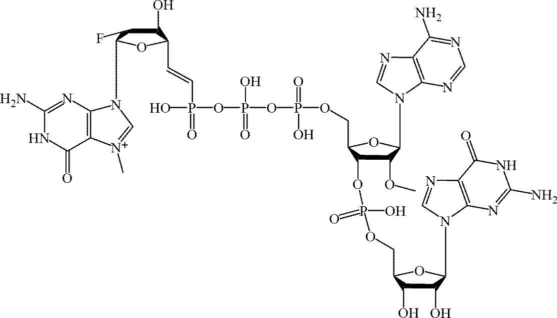

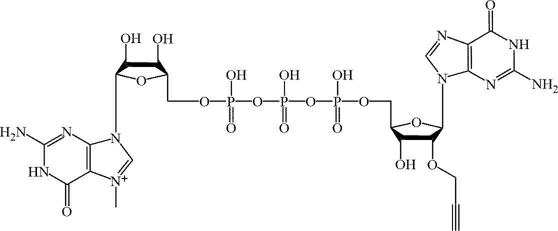

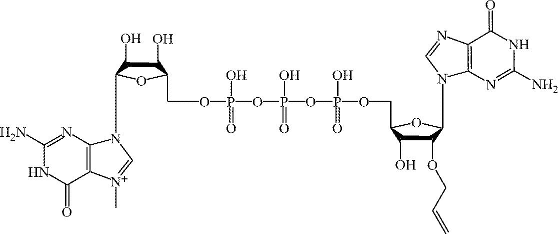

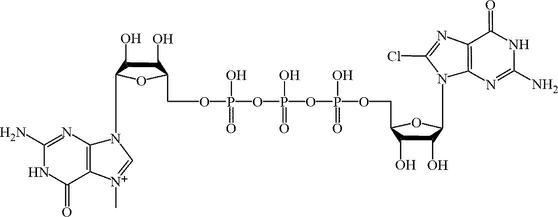

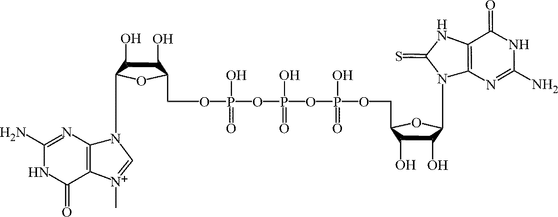

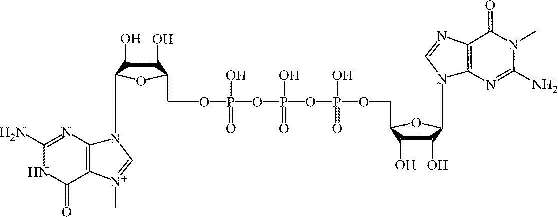

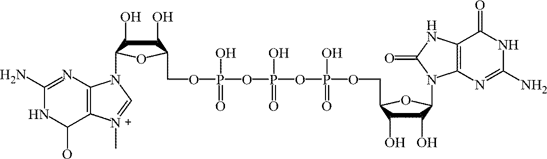

### EXAMPLES

### Method of Synthesis

### 5′-Cap Structures Synthesis Protocol

### Example 1

Mono-phosphorylation of modified nucleoside (3′-moeGMP)

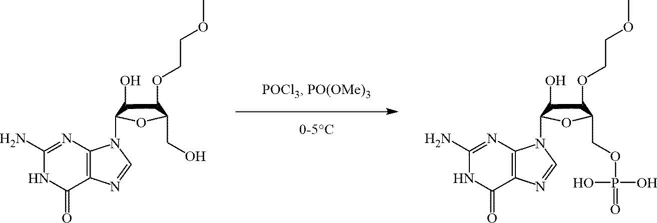

In an Ar flushed 4 mL glass vial with septa filled with 3′-moeG (100 mg, 0.29 mmol), trimethylphosphate (TMP, 1.0 mL) was added and the suspension was cooled to 0° C. in an ice bath for 30 min before POCl₃ (35 uL, 1.3 eq) was added via a dried syringe. The whole mixture turned clear within 1 min. Stirring was continued in an ice bath for 3 h before it was quenched with water (30 mL) and 15 mL TEAB (1.0 M) was added to adjust the pH to 6.

The mixture was washed with DCM and the organic layer discarded (20 mL x2). The resulting aqueous layer was diluted to 250 mL and loaded onto a 100 mL DEAE column and eluted with a 0-25% water/1.0 M DEAB gradient. Product fractions were combined, evaporated, and freeze-dried as triethylammonium white solids (0.28 mmol, 95%). ¹H NMR (Deuterium Oxide, 400 Hz) δ 8.13 (s, 1H), 5.96 (d, J = 8 Hz, 1H), 4.40 (m, 1H), 4.2 (m, 1H), 4.0 (m, 2H), 3.8 (m, 2H), 3.7 (m, 2H), 3.40 (s, 3H), 3.2 (q, J = 9 Hz, 12H), 1.2 (t, J = 9, 18H). 2′-F-araGMP was synthesized by a similar method.

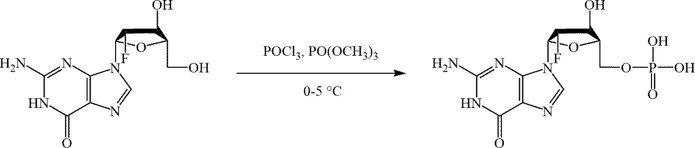

In an Ar flushed 4 mL glass vial with septa filled with 2′-Fluoro-araG (100 mg, 0.35 mmol), trimethylphosphate (TMP, 1.0 mL) was added and the suspension was cooled to 0° C. in an ice bath for 30 min before POCl₃ (43 uL, 1.3 eq) was added via a dried syringe. The whole mixture turned clear within 1 min. Stirring was continued in an ice bath for 3 h before it was quenched with water (30 mL) and 15 mL TEAB (1.0 M) was added to adjust the pH to 6. The mixture was washed with DCM and the organic layer discarded (20 mL x2). The resulting aqueous layer was diluted to 250 mL and loaded on a 100 mL DEAE column and eluted with a 0-25% water/1.0 M DEAB gradient. Product fractions were combined, evaporated, and freeze-dried as triethylammonium white solids (0.33 mmol, 95%). ¹H NMR (Deuterium Oxide, 400 Hz) δ 8.06 (s, 1H), 6.27-6.32 (m, 1H), 5.20-5.35 (m, 1H), 4.63-4.69 (m, 1H), 4.04-4.19 (m, 3H).

thio-G) monophosphate was synthesized by a similar method. (240 mg, 45%) ¹H-NMR (Deuterium Oxide, 400 Hz) δ 8.2 (s, 1H), 5.9 (d, J = 6.0 Hz, 1H), 4.4 (m, 1H), 4.2 (m, 1H), 3.9 (m, 2H), 3.1 (q, J = 8 Hz, 6H), 1.1 (t, J = 8 Hz, 9H) ppm. ³¹P NMR (Deuterium Oxide, 162 Hz) δ 2.7 (s, IP) ppm.

### Example 2

Pyro-phosphorylation of modified nucleosides through one-pot syntheses. (3′moeGDP)

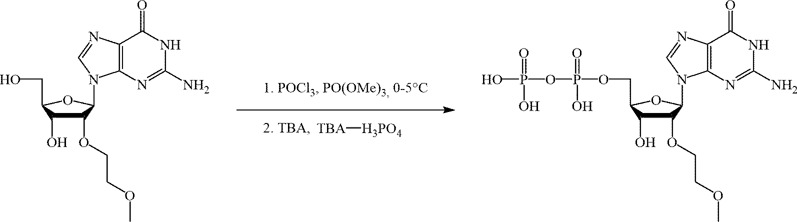

A modified literature protocol was adopted⁹. In a 0° C. suspension of 2′moeG (1.0 mmol) in TMP (2.0 mL), POCl₃ was added, and the mixture was warmed up at rt for 30 min before the starting material was completely dissolved. The mixture was cooled to 0° C. and tributylamine (TBA, 0.70 mmol) was added followed by a solution of H₃PO₄-TBA salt in anhydrous DMF (5.0 mL x 1 M). After 5 min of stirring at 0° C., it was quenched with 60 mL TEAB (0.5 M) buffer for 30 min. The mixture was diluted to 500 mL and purified on a 100 mL DEAE column. The product fractions were combined, concentrated, and freeze dried as triethylammonium white waxy solids: 0.44 g (0.55 mmol, 55%). The product contained 20% mol of H₃PO₄ and can be used as is. An analytical sample was obtained by further purification on HPLC to yield ammonium salt. ¹H NMR (Deuterium Oxide, 400 Hz) δ 8.14 (s, 1H), 5.98 (d, J =8 Hz, 1H), 4.7 (m, 2H), 4.3 (m, 1H), 4.2 (m, 2H), 3.8 (m, 1H), 3.7 (m, 1H), 3.6 (m, 2H), 3.30 (s, 3H). ³¹P NMR (Deuterium Oxide, 162 Hz) δ -9.2 (d, J = 20 Hz, 1P), -11.2 (d, J = 20 Hz, 1P).

### Example 3

7-alkylation of guanosine monophosphate. 7-(2-F-ethyl) GMP

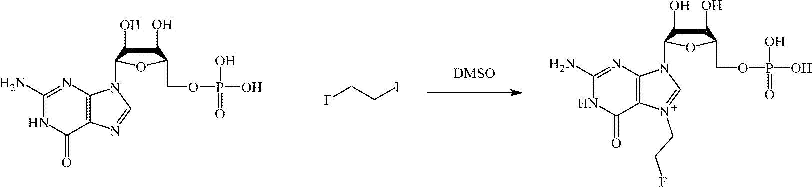

Guanosine monophosphate sodium salt (0.50 mmol) was suspended in DMSO (4 mL), 0.43 g 1-fluoro-2-iodoethane (2.5 mmol) was added and the mixture was maintained with stirring at 55° C. for 48 h. The mixture was diluted with water (50 mL) and washed with DCM (25 mL x 2). The aqueous layer was purified by 100 mL DEAE column. The product fractions were combined, concentrated, and freeze dried as white powder (35 umol, 7%). ¹H NMR (Deuterium Oxide, 400 Hz) δ 6.13 (d, J = 4 Hz, 1H), 5.0 (m, 1H), 4.7-4.9 (m, 4H), 4.5 (m, 1H), 4.4 (m, 1H), 4.2 (m, 1H), 4.1 (m,1H), 3.2 (q, J = 9 Hz, 6H), 1.2 (t, J = 9, 9H). 7 m-2′-F-araGMP was synthesized by a similar method.

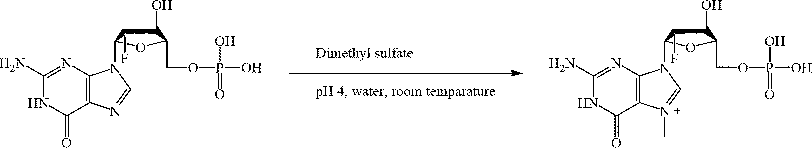

Guanosine monophosphate triethylammonium salt (0.92 mmol) was suspended in water (50 mL) and the pH of the resulting solution was adjusted to 4.0 using acetic acid. 1.3 mL (13.8 mmol) of dimethyl sulfate was added dropwise over a period of 2 hr. As methylation proceeds, the pH decreases but was readjusted back to 4.0 using 1.0 N NaOH solution. The reaction mixture was stirred at room temperature for 3 hr. The mixture was diluted with water (50 mL) and washed with DCM (100 mL x 2). The aqueous layer was purified on a 100 mL DEAE column. The product fractions were combined, concentrated, and freeze dried as white powder (0.9 mmol, 90%). ¹H NMR (Deuterium Oxide, 400 Hz) δ 6.51-6.55 (m, 1H), 5.35-5.50 (m, 1H), 4.68-4.74 (m, 1H), 4.30-4.4.33 (m, 1H), 4.11-4.4.20 (m, 5H). ³¹P NMR (Deuterium Oxide, 162 Hz) δ 3.85 (s, IP).

7mXMP was synthesized by a similar method. (0.15 mmol, 30%) ¹H-NMR (Deuterium Oxide, 400 Hz) δ 9.0 (s, 1H), 6.0 (d, J = 3.6 Hz, 1H), 4.6 (m, 1H), 4.4 (m, 2H), 4.1-4.2 (m, 2H), 4.0 (s, 3H) ppm. ³¹P-NMR (Deuterium Oxide, 162 Hz) δ 0.02 (s, IP).

7mIDP was synthesized by a similar method. (0.21 mmol, 34%) ¹H-NMR (Deuterium Oxide, 400 Hz) δ 8.2 (s, 1H), 6.2 (d, J = 2.8 Hz, 1H), 4.6 (m, 1H), 4.5 (m, 1H), 4.3 (m, 2H), 4.2 (m, 1H), 3.8 (s, 1H), 3.1 (q, J = 8 Hz, 18H), 1.2 (t, J = 8 Hz, 27H). ³¹P-NMR

(Deuterium Oxide, 162 Hz) δ -7.6 (d, J = 21 Hz, 1P), -11.1 (d, J = 21 Hz, 1P).

### Example 4

Activation of modified nucleoside phosphate (Im-7mGDP)

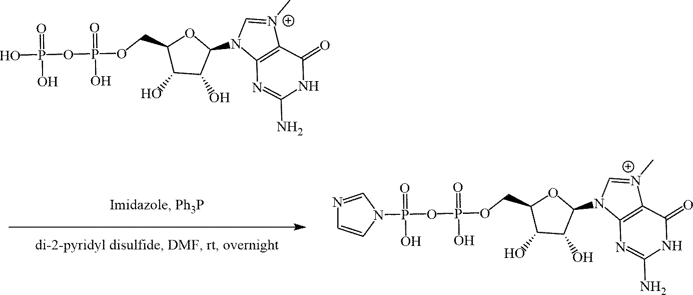

To a solution of 7mGDP as TEA salt (1.8 g, 3.9 mmol) in 54 mL of dimethyl formamide under an inert atmosphere of nitrogen was added, in sequence, imidazole (2.7 g, 39.5 mmol, 10.0 eq), triethylamine (0.8 g, 7.9 mmol, 2.0 eq), triphenyl phosphate (3.1 g, 11.8 mmol, 3.0 eq), and di-2-pyridyl disulfide (2.6 g, 11.8 mmol, 3.0 eq) at room temperature. The resulting solution was stirred overnight at room temperature. Then 120 mL acetone (containing 66 mmol/L sodium perchlorate) was added dropwise and stirred at 0° C. for 30 min. The resulting mixture was filtered and washed with acetone to yield 1.0567 g (53%) of 7mGDP-Im as a white solid. ¹H NMR (400 MHz, Deuterium Oxide) δ 7.87 (s, 1H), 7.53 (s, 1H), 7.25 (s, 1H), 6.95 (s, 1H), 5.96 (t, J = 3.7 Hz, 1H), 4.52 (d, J = 4.2 Hz, 1H), 4.28 (d, J = 4.5 Hz, 2H), 4.15 (s, 1H), 3.98 (d, J = 4.8 Hz, 4H). ³¹P NMR (162 MHz, Deuterium Oxide) δ -11.9 (d, J = 20 Hz, 1P), -19.9 (d, J = 20 Hz, 2P).

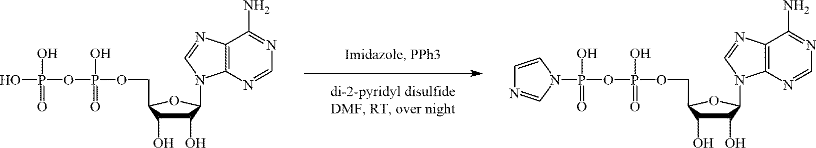

To a solution of ADP as TEA salt (147 mg, 0.2 mmol) in 7.5 mL of dimethyl formamide under an inert atmosphere of nitrogen was added, in sequence, imidazole (163.3 mg, 2.4 mmol, 12.0 eq), triethylamine (40.4 mg, 0.4 mmol, 2.0 eq) triphenyl phosphate (314 mg, 1.2 mmol, 6.0 eq), and di-2-pyridyl disulfide (264 mg, 1.2 mmol, 6.0 eq) at room temperature. The resulting solution was stirred overnight at room temperature. Then 120 mL acetone (containing 66 mmol/L sodium perchlorate) was added dropwise and stirred at 0° C. for 30 min. The resulting mixture was filtered and washed with acetone to yield 0.13 mmol of ADP-Im as a white solid.

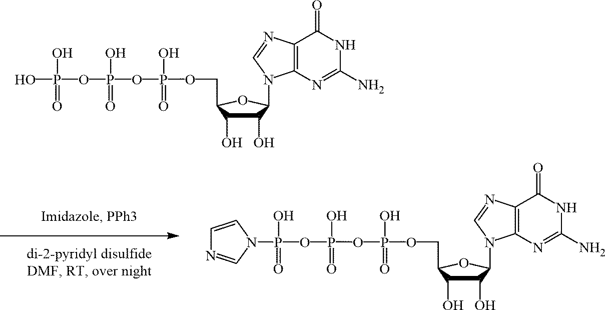

To a solution of GTP as TEA salt (0.413 mg, 0.5 mmol) in 7.5 mL of dimethyl formamide under an inert atmosphere of nitrogen was added, in sequence, imidazole (0.408 mg, 6 mmol, 12.0 eq), triethylamine (101 mg, 1 mmol, 2.0 eq), triphenyl phosphate (786 mg, 3 mmol, 6.0 eq) and di-2-pyridyl disulfide (660 mg, 3 mmol, 6.0 eq) at room temperature. The resulting solution was stirred overnight at room temperature. Then 120 mL acetone (containing 66 mmol/L sodium perchlorate) was added dropwise and stirred at 0° C. for 30 min. The resulting mixture was filtered and washed with acetone to yield 288 mg (90%) of GTP-Im as a white solid.

7mIDP-Im was synthesized by a similar method. ¹H-NMR (Deuterium Oxide, 400 Hz) δ 8.2 (s, 1H), 7.9 (s, 1H), 7.3 (s, 1H), 6.9 (s, 1H), 6.1 (s, 1H), 4.5 (m, 1H), 4.3 (m, 2H), 4.2 (m, 1H), 4.1 (s, 3H), 4.0 (m, 1H) ppm. ³¹P-NMR (Deuterium Oxide, 162 Hz) δ -11.9 (d, J = 20 Hz, 1P).

### Example 5

Ligation production of modified 5′-cap analogs via zinc catalysis. (7-(2-F-ethyl)-GpppG (13)

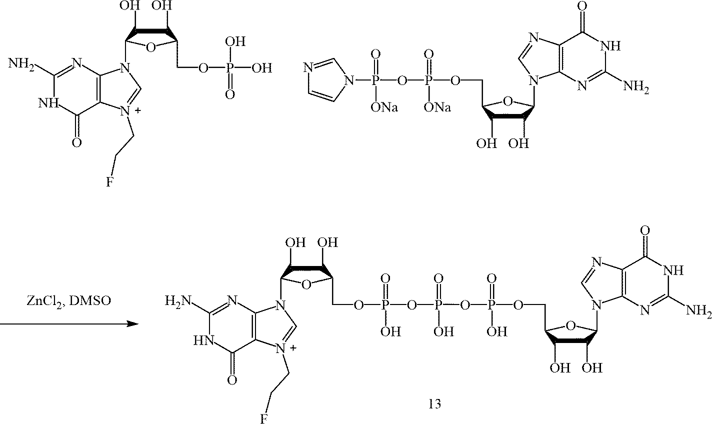

In a 4 mL vial, to a mixture of GDP-Imidazole (14 mg, 25 µmol), 7-alkylated GMP-TEA (25 µmol) and zinc chloride (68 mg, 500 µmol) was added dry DMSO (500 µL), and then the whole mixture was stirred at 35° C. for 24 h. The reaction mixture was diluted with 50 mL water and the mixture was treated with resin-bounded Zn scavenger (QuadraPure IDA, 0.5 g). The solution was filtered, concentrated, and purified by prep-HPLC resulting in ammonium salt (4.8 µmol). The ammonium salt was freeze dried twice and converted to sodium salt by precipitation from NaClO₄ solution in acetone (50 mg/mL, 1 mL) followed by acetone wash resulting in a white solid (4.1 µmol, 16%). ¹H NMR (Deuterium Oxide, 400 Hz) δ 8.06 (s, 1H), 5.95 (d, J = 4 Hz, 1H), 5.84 (d, J = 8 Hz, 1H), 5.0 (m, 1H), 4.7-4.9 (m, 4H), 4.6 (m, 1H), 4.5 (m, 2H), 4.2-4.5 (m, 6H). ³¹P NMR (Deuterium Oxide, 162 Hz) δ -10.8 (d, J = 20 Hz, 2P), -22.4 (t, J = 20 Hz, 1P). MS (m/z): found 835.2 [M⁺], calculated 835.1 [M⁺]

7m(2′-moe)GpppG (1) was synthesized by a similar method. ¹H-NMR (D₂O, 400 MHz) δ 7.91 (s, 1H), 5.86 (d, 1H), 5.70 (d, 1H), 4.57 (t, 1H), 4.40-4.10(m, 9H), 3.98 (s, 3H), 3.85-3.82 (m, 2H), 3.58 (m 2H), 3.28 (s, 3H) ; ³¹P-NMR (D₂O, 162 MHz) δ -11.62 (d, 2P), -23.13 (d, 1P), MS (m/z): found 861.1 [M⁺], calculated 861.1 [M⁺]

7m(3′-moe)GpppG (2) was synthesized by a similar method. ¹H NMR (Deuterium Oxide, 400 MHz) δ 8.02 (s, 1H), 5.90 (d, J = 4 Hz, 1H), 5.81 (d, J = 8 Hz, 1H), 4.7 (m, 2H), 4.5 (m, 2H), 4.4 (m, 2H), 4.2-4.4 (m, 4H), 4.09 (s, 3H), 3.8 (m, 2H), 3.7 (m, 2H), 3.42 (s, 3H). ³¹P NMR (Deuterium Oxide, 162 Hz) δ -11.0 (d, J = 20 Hz, 1P), -11.2 (d, J = 20 Hz, 1P), -22.4 (t, J = 20 Hz, 1P). MS (m/z): found 861.0 [M⁺], calculated 861.1 [M⁺]

7m(2′-F-araG)pppG (6) was synthesized by a similar method. ¹H NMR (Deuterium Oxide, 400 MHz) δ 8.01 (s, 1H), 6.26 (d, J = 8 Hz, 1H), 5.80 (d, J = 4 Hz, 1H), 5.24-5.35 (m, 1H), 4.63-4.65 (m, 2H), 4.48-4.49 (m, 1H), 4.26-4.36 (m, 6H), 4.09 (m, 3H). ³¹P NMR (Deuterium Oxide, 162 Hz) δ -10.9 (d, J = 20 Hz, 2P), -22.6 (t, J = 20 Hz, 1P). MS (m/z): found 806.0 [M⁺], calculated 806.1 [M⁺]

7 m(2′-F-araG)ppp(2′moeG) (7) was synthesized by a similar method. ¹H NMR (Deuterium Oxide, 400 MHz) δ 7.99 (s, 1H), 6.23 (d, J = 8 Hz, 1H), 5.81 (d, J = 4 Hz, 1H), 5.22-5.35 (m, 1H), 4.47-4.66 (m, 3H), 4.25-4.4 (m, 6H), 4.08 (m, 3H), 3.6-3.8 (m, 2H), 3.4-3.54 (m, 2H), 3.19 (s, 3H). ³¹P NMR (Deuterium Oxide, 162 Hz) δ -11.6 (d, J = 20 Hz, 2P), -23.2 (t, J = 20 Hz, 1P). MS (m/z): found 863.0 [M⁺], calculated 863.1 [M⁺]

7-e-GpppG (11) was synthesized by a similar method. ¹H NMR (Deuterium Oxide, 400 MHz) δ 8.03 (s, 1H), 5.89 (d, J = 4 Hz, 1H), 5.80 (d, J = 8 Hz, 1H), 4.7 (m, 1H), 4.3-4.6 (m, 7H), 1.5 (t, J = 8 Hz, 3H). ³¹P NMR (Deuterium Oxide, 162 Hz) δ -11.6 (d, J = 20 Hz, 2P), -23.2 (t, J = 20 Hz, 1P). MS (m/z): found 817.2 [M⁺], calculated 817.1 [M⁺]

7-iPr-GpppG (12) was synthesized by a similar method. ¹H NMR (Deuterium Oxide, 400 MHz) δ 8.07 (s, 1H), 5.89 (d, J = 4 Hz, 1H), 5.82 (d, J = 8 Hz, 1H), 5.1 (m, 1H), 4.8 (m, 1H), 4.6 (m, 1H), 4.5 (m, 2H), 4.2-4.4 (m, 6H), 1.6 (t, J = 8 Hz, 6H). ³¹P NMR (Deuterium Oxide, 162 Hz) δ -10.9 (m, 2P), -22.6 (t, J = 20 Hz, 1P). MS (m/z): found 832.0 [M⁺], calculated 831.1 [M⁺]

7-(cPr-methyl)-GpppG (14) was synthesized by a similar method. ¹H NMR (Deuterium Oxide, 400 MHz) δ 8.08 (s, 1H), 5.96 (d, J = 4 Hz, 1H), 5.84 (d, J = 8 Hz, 1H), 4.8 (m, 1H), 4.7 (m, 1H), 4.5 (m, 2H), 4.2-4.4 (m, 8H), 1.5 (m, 1H), 0.7 (m, 2H), 0.5 (m, 2H). ³¹P NMR (Deuterium Oxide, 162 Hz) δ -11.6 (d, J = 20 Hz, 2P), -23.2 (t, J = 20 Hz, 1P). MS (m/z): found 843.2 [M⁺], calculated 843.1 [M⁺]

7mIpppG (17) was synthesized by a similar method: ¹H NMR (Deuterium Oxide, 400 Hz) δ 8.2 (s, 1H), 8.0 (m, 1H), 6.0 (s, 1H), 5.7 (s, 1H), 4.2-4.4 (m, 7H), 4.1-4.2 (m, 6H) ppm. ³¹P-NMR (Deuterium Oxide, 162 Hz) δ -11.5 (d, J = 20 Hz, 2P), -23.2 (t, J = 22 Hz, 1P) ppm. MS (m/z): found 786.6 [M-2H]⁻, calculated 788.1 [M⁺]

7mIppp(2′-moeA) (18) was synthesized by a similar method: ¹H NMR (Deuterium Oxide, 400 Hz) δ 8.3 (s, 1H), 8.1 (s, 1H), 8.0 (s, 1H), 5.9 (m, 2H), 4.5 (m, 1H), 4.4-4.5 (m, 2H), 4.3-4.4 (m, 4H), 4.2 (m, 3H), 4.0 (s, 1H), 3.7 (m, 1H), 3.6 (m, 1H), 3.4 (m, 2H), 3.0 (s, 3H) ppm. ³¹P-NMR (Deuterium Oxide, 162 Hz) δ -11.5 (d, J = 16 Hz, 2P), -23.2 (t, J = 18 Hz, 1P) ppm. MS (m/z): found 828.4 [M-H]⁻, calculated 829.1 [M⁺]

7mIpppthio-G) (19) was synthesized by a similar method. ¹H-NMR (Deuterium Oxide, 400 Hz) δ 8.1 (s, 1H), 8.0 (m, 1H), 6.0 (d, J = 3.6 Hz, 1H), 5.7 (d, J = 6.4 Hz, 1H), 4.6 (m, 1H), 4.4 (m, 2H), 4.3-4.4 (m, 2H), 4.2 (m, 1H), 4.1-4.2 (m, 3H), 4.1 (s, 3H) ppm. ³¹P-NMR (Deuterium Oxide, 162 Hz) δ -11.5 (d, J = 18 Hz, 2P), -23.1 (t, J = 18 Hz, 1P) ppm. MS (m/z): found 804.4 [M⁺], calculated 804.1.[M⁺].

7mXpppG (20) was synthesized by a similar method. ¹H NMR (Deuterium Oxide, 400 Hz) δ 8.8 (s, 1H), 8.0 (s, 1H), 6.8 (s, 1H), 5.8 (d, J = 5.6 Hz, 1H), 4.6 (m, 1H), 4.4 (m, 2H), 4.2-4.4 (m, 4H), 4.1-4.2 (m, 3H), 3.9 (s, 3H) ppm. ³¹P-NMR (Deuterium Oxide, 162 Hz) δ -11.5 (d, J = 16 Hz, 2P), -23.1 (t, J = 16 Hz, 1P) ppm. MS (m/z): found 804.4 [M⁺], calculated 804.1 [M⁺]

### Example 6

Ligation production of modified 5′-cap analogs via magnesium catalysis 7-(5′-vinyl-G)pppG (26)

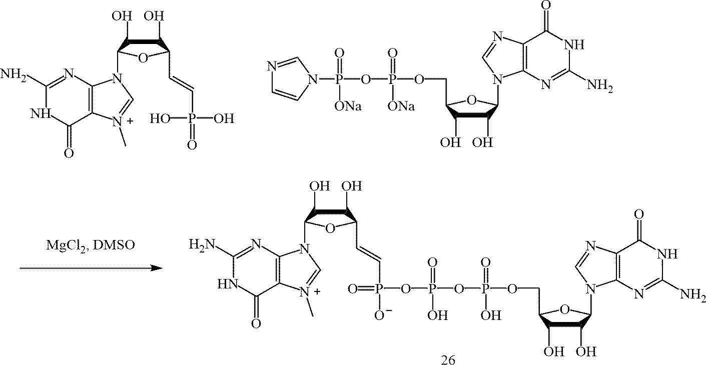

A modified literature protocol was adopted ¹⁰. In a 4 mL vial, to a mixture of GDP-Imidazole (14 mg, 25 µmol), modified GMP-TEA (25 µmol) and magnesium chloride (24 mg, 250 µmol) was added dry DMSO (500 µL) and then the whole mixture was stirred at rt for 72 h. The reaction mixture was added into (0.05 M 5 mL) EDTA-TEA, diluted to 40 mL and purified on a 20 mL DEAE column. The major peak was concentrated and further purified by prep-HPLC resulting in ammonium salt (5.1 mg). The ammonium salt was freeze dried twice and converted to sodium salt by precipitation from NaClO₄ solution in acetone (50 mg/mL, 1 mL) followed by acetone wash resulting in a white solid (2.2 µmol). ¹H NMR (Deuterium Oxide, 400 Hz) δ 7.99 (s, 1H), 6.7 (m, 1H), 6.3 (t, J = 20 Hz), 5.88 (d, J = 4 Hz, 1H), 5.78 (d, J = 8 Hz, 1H), 4.7-4.9 (m, 2H), 4.6 (m, 1H), 4.5 (m, 1H), 4.2-4.4 (m, 4H), 4.08 (s, 3H). ³¹P NMR (Deuterium Oxide, 162 Hz) δ 4.2 (d, J = 18 Hz, 1P), -11.0 (d, J = 18 Hz, 1P), -22.6 (t, J = 18 Hz, 1P). MS (m/z): found 799.0 [M⁺], calculated 799.1 [M⁺]

7mGppp(5′vinyl-G) (27) was synthesized by a similar method. ¹H NMR (Deuterium Oxide, 400 Hz) δ 7.89 (s, 1H), 6.7 (dt, J₁ = 16 Hz, J₂= 4 Hz, 1H), 6.3 (t, J = 16 Hz, 1H), 5.7 (m, 2H), 4.7 (m, 1H), 4.6 (m, 1H), 4.2-4.5 (m, 6H), 4.05 (s, 3H). ³¹P NMR (Deuterium Oxide, 162 Hz) δ 4.5 (d, J = 20 Hz, 1P), -11.0 (d, J = 20 Hz, 1P), -22.5 (t, J = 20 Hz, 1P). MS (m/z): found 799.0 [M⁺], calculated 799.1 [M⁺]

7mGppp_{CH2}G (29) was synthesized by a similar method. ¹H NMR (Deuterium Oxide, 400 Hz) δ 7.91 (s, 1H), 5.9 (d, J = 4 Hz, 1H), 5.7 (d, J = 8 Hz, 1H), 4.7 (m, 1H), 4.5 (m, 1H), 4.3-4.5 (m, 5H), 4.2 (m, 1H), 4.14 (s, 3H), 2.0 (m, 4H). ³¹P NMR (Deuterium Oxide, 162 Hz) δ 18.3 (d, J = 20 Hz, 1P), -11.6 (d, J = 20 Hz, 1P), -23.2 (t, J = 20 Hz, 1P). MS (m/z): found 801.4 [M⁺], calculated 801.1 [M⁺]

### Example 7

Solid phase syntheses of 5′-phosphate of dinucleotide p(2′-omeA)rG (41).

Commercially available universal base controlled-pore glass (CPG) solid support 57 mg (89.3 µmol/g) with a pore size of 500 Å was placed in a column fitted with a filter and the column was installed in an H-6 DNA/RNA/LNA synthesizer (K&A Laborgeraete GbR). Synthesis of dinucleotides was performed using ten tubes at 5 µmol scale on 5′-O-DMT-2′O-TBDMS-rG^{iBu} CPG (500A) solid support. In the coupling step, excessive 5′-O-DMT-2′-O-TBDMS/2′-O-Me-3′-O-phosphoramidite (rAₒₘₑ^{Bz}, rG^{iBu}, from NovoCom Bio), or biscyanoethyl phosphoramidite were recirculated through the column for 14 min. A solution of 3% (v/v) dichloroacetic acid in toluene was used as a detritylation reagent and 0.05 M iodine in pyridine was used for oxidation, 10% (v/v) acetic anhydride in acetonitrile was used as Cap A and a mixture of 14% (v/v) N-methylimidazole and 10% (v/v) pyridine in acetonitrile was used as Cap B. Finally, the solid support was washed with acetonitrile and dried with argon. The product was cleaved from the solid support and deprotected with ethanolic ammonia (ammonium hydroxide 1:3 v/v; 2 mL, 55° C., 6 h), evaporated to dryness and redissolved in DMSO (500 µl). The TBDMS groups were removed using triethylammonium trihydrofluoride (TEA·3HF; 620 ul, 65° C., 2.5 h), and then the mixture was cooled down and diluted with 0.10 M TEAB (15 ml). The product was isolated by ionexchange chromatography on a DEAE Sephadex column (gradient elution 0-0.9 M TEAB) resulting in, after evaporation, triethylammonium salt of p(2′-omeA)rG dinucleotide (19 mg, 53%). ¹H NMR (400 MHz, D₂O): δ = 8.35 (s, 1H), 8.12 (s, 1H), 7.89 (s, 1H), 6.02 (d, J = 4.0 Hz, 1H), 5.76 (d, J = 8.0 Hz, 1H), 4.70 (t, J = 4.0 Hz, 1H), 4.47 (t, J = 4.0 Hz, 2H), 4.42 (t, J = 4.0 Hz, 1H), 4.24 (b, 2H), 4.14 (d, J = 8.0 Hz, 2H), 3.59 (t, J = 4.0 Hz, 1H), 3.37 (t, J = 4.0 Hz, 1H), 3.05 (s, 3H); ³¹P NMR (162 MHz, D₂O, 25° C.): δ = 0.46 (s, 1P), δ = 0.93 (s, 1P) ppm. MS (m/z): found 705 [M-H]⁻, Calculated 706.

p(2′-moeA)rG (42) was synthesized by a similar method: (16 mg, 43%): ¹H-NMR (400 MHz, D₂O): δ = 8.35 (s, 1H), 8.12 (s, 1H), 7.89 (s, 1H), 6.01 (d, J = 8.0 Hz, 1H), 5.76 (d, J = 8.0 Hz, 1H), 4.70 (t, J = 4.0 Hz, 1H), 4.47 (t, J = 4.0 Hz, 2H), 4.41 (t, J = 4.0 Hz, 1H), 4.24 (b, 2H), 4.14 (d, J = 8.0 Hz, 2H), 3.59 (t, J = 4.0 Hz, 1H), 3.37 (t, J = 4.0 Hz, 1H), 3.00 (t, J = 4.0 Hz, 4H), 2.64 (s, 3H); ³¹P-NMR (162 MHz, D₂O): δ = 0.22 (s, 1P), δ = 0.84 (s, 1P) ppm. MS (m/z) found 749 [M-H]⁻Calculated 750.

p(2′-moeA)p(2′-moeG) (43) was synthesized by a similar method: (2.1 mg, 59%). ¹H-NMR (400 MHz, D₂O): δ = 8.57 (s, 1H), 8.13 (s, 1H), 7.89 (s, 1H), 6.08 (d, J = 8.0 Hz, 1H), 5.84 (d, J = 4.0 Hz, 1H), 4.84-4.87 (m, 1H), 4.63(t, J = 4.0 Hz, 2H), 4.52(t, J = 4.0 Hz, 1H), 4.85 (b, 1H), 4.47 (b, 1H), 4.26 (b, 1H), 4.12 (d, J = 8.0 Hz, 1H), 3.91 (t, J = 4.0 Hz, 2H), 3.69-3.76 (m, 2H), 3.50-3.62 (m, 2H), 3.39-3.43 (m, 2H), 3.20-3.21 (m, 2H), 3.09 (s, 3H), 2.97 (s, 3H); ³¹P-NMR (162 MHz, D₂O): δ = 3.7 (s, 1P) ppm, δ = 0.7 (s, 1P) ppm; MS (m/z): found 807 [M-H]⁻, Calculated 808.

### Example 8

Ligation reactions with modified mononucleotide tail. 7mGppp(2′-moeA) (53)

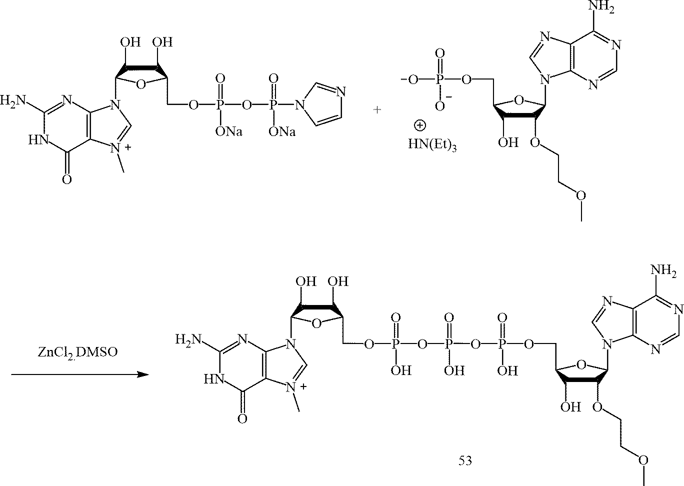

In a lOmL bottle, a mixture of Im-7mGDP sodium salt (6 mg, 11 µmol), 2′-moeAMP TEA salt (7 mg, 17 µmol), DMSO (1.0 mL) and ZnCl₂ (20 mg, 147 µmol) was stirred at room temperature for 48h. The mixture was quenched by EDTA-2Na solution (55 mg, 150 µmol) in 10 mL water. The solution was diluted with 100 mL water, the pH was adjusted to 6.5 using TEAB buffer and loaded onto the column (GE DEAE Fast Flow) at a flow rate of 5 mL/min with UV detection at 260 nm. The column was eluted with a linear gradient of 0 to 1 M TEAB buffer, and the fractions were collected, evaporated and co-evaporated with 100 mL water three times. Purification by Prep-HPLC gave product (3 mg, 31%). ¹H-NMR (D₂O, 400 MHz) δ 8.32 (s, 1H), 8.11 (s, 1H), 5.97 (d, 1H), 5.78 (d, 1H), 4.51-4.29 (m, 10H), 3.95 (s, 3H), 3.70-3.56 (m, 2H), 3.40 (m 2H), 3.09 (s, 3H) ppm; ³¹P-NMR (D₂O,162 MHz) δ -11.63 (d, 2P), -23.18 (d, 1P) ppm, MS (m/z): found 845.0 [M-H]⁺, calculated 845.1

7mGppp(6mA) (50) was synthesized by a similar method. ¹H NMR (Deuterium Oxide, 400 Hz) δ 8.39 (s, 1H), 8.20 (s, 1H), 6.03 (d, J = 8 Hz, 1H), 5.85 (d, J = 4 Hz, 1H), 4.7 (m, 1H), 4.5 (m, 1H), 4.2-4.5 (m, 8H), 3.99 (s, 3H), 3.11 (s, 3H) ppm. ³¹P NMR (Deuterium Oxide, 162 Hz) δ -11.0 (m, 2P), -22.6 (t, J = 20 Hz, 1P) ppm. MS (m/z): found 801.0 [M⁺], calculated 801.1 [M⁺]

7mGpppthio-G) (51) was synthesized by a similar method. ¹H NMR (Deuterium Oxide, 400 Hz) δ 8.0 (s, 1H), 5.8 (d, J = 3.2 Hz, 1H), 5.7 (d, J = 4.8 Hz, 1H), 4.5 (m, 1H), 4.4 (m, 1H), 4.3(m, 1H), 4.2-4.3 (m, 3H), 4.1-4.2 (m, 3H) ppm. ³¹P-NMR (Deuterium Oxide, 162 Hz) δ -11.6 (d, J = 18 Hz, 2P), -23.2 (t, J = 18 Hz, 1P) ppm. MS (m/z): found 819.3 [M+H]⁺, calculated 818.1

7mGppp(2′moeG) (52) was synthesized by a similar method: (4 mg,22%). ¹H-NMR (D₂O, 400 MHz) δ 7.89 (s, 1H), 5.80 (d, 1H), 5.75 (d, 1H), 4.51-4.44 (m, 3H), 4.40-4.12 (m, 7H), 3.98 (s, 3H), 3.74-3.56 (m, 2H), 3.40 (m 2H), 3.11 (s, 3H) ; ³¹P-NMR (D₂O,162 MHz) δ -11.6 (d, J = 20 Hz, 2P), -23.2 (t, J = 20 Hz, 1P), MS (m/z): found 861.0 [M-H]⁺, calculated 861.1

### Example 9

Ligation production of trinucleotide modified 5′-cap analogs 7mGppp(2′-moeA)rG (55)

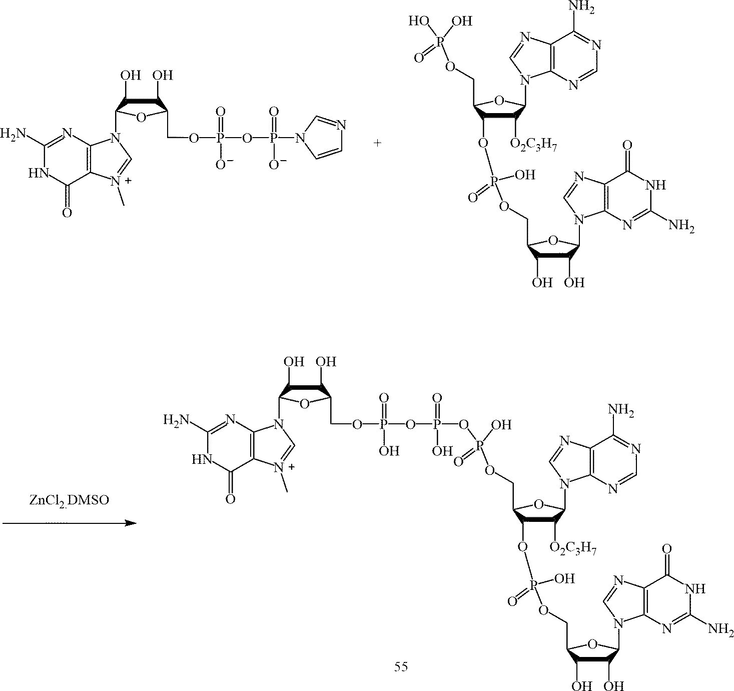

In a 10 mL bottle, Im-7mGDP sodium salt (13 mg, 25 µmol), 5′p-(2′-moeA)rG TEA salt (11.8 mg, 14.6 µmol), DMSO (1.5 mL), and ZnCl₂ (72 mg, 500 µmol) was added. The reaction was stirred at room temperature for 24 h and was subsequently quenched by EDTA-2 Na (185 mg, 550 µmol) in water (10 mL). The solution was diluted to 100 mL with water and the pH was adjusted to 6.5 using TEAB buffer. The sample solution was loaded onto the column (GE DEAE Fast Flow) at a flow rate of 5 mL/min with UV detection at 260 nm; and the column was eluted with a linear gradient of 0 to 1 M TEAB buffer, and the fractions were collected and evaporated using a rotary evaporator at 35° C. Then the resulting residue was co-evaporated with 100 mL water three times. Freeze-drying yielded crude product which was purified by prep-HPLC to get 2 mg product (yield 17%). ¹H NMR (Deuterium Oxide, 400 MHz) δ 8.29 (s, 1H), 8.06 (s, 1H), 7.91 (s, 1H), 5.92 (d, 1H), 5.78 (m, 2H), 4.48-4.13 (m, 15H), 3.95 (s, 3H), 3.67(m, 1H), 3.47(m, 1H), 3.32-3.14(m, 2H), 2.90(s, 3H); ³¹P NMR (Deuterium Oxide, 162 MHz) δ-11.70 (m, 2P), -22.64 (m, 1P), -0.86 (s, 1P). MS (m/z): found 1190[M-H]+. 596[M/2]+, calculated 1190.2

7mGppp-(2′-moeA)p(2′-moeG) (56) was synthesized by a similar method. ¹H NMR (Deuterium Oxide, 400 MHz) δ 8.94 (s, 1H), 832 (s, 1H), 8.09 (s, 1H), 7.86 (s, 1H), 5.94 (d, 1H), 5.80 (m, 2H), 4.52-4.12 (m, 15H), 3.96 (s, 3H), 3.76-3.68 (m, 2H), 3.64-3.58 (m, 1H), 3.53-3.49 (m, 1H), 3.44-3.41(m, 2H), 3.35-3.24 (m, 2H), 3.12 (s, 3H), 3.03 (s, 3); ³¹P NMR (Deuterium Oxide, 162 MHz) δ-11.35 (m, 2P), -22.58 (m, 1P), -0.82 (s, 1P), MS (m/z): found 625[M/2]⁺, calculated 1248.2

### Example 10

Synthesis of 7mGppp(locked)Ap(locked)ApG (GL-Cap 1) Synthesis of p(locked)Ap(locked)ApG

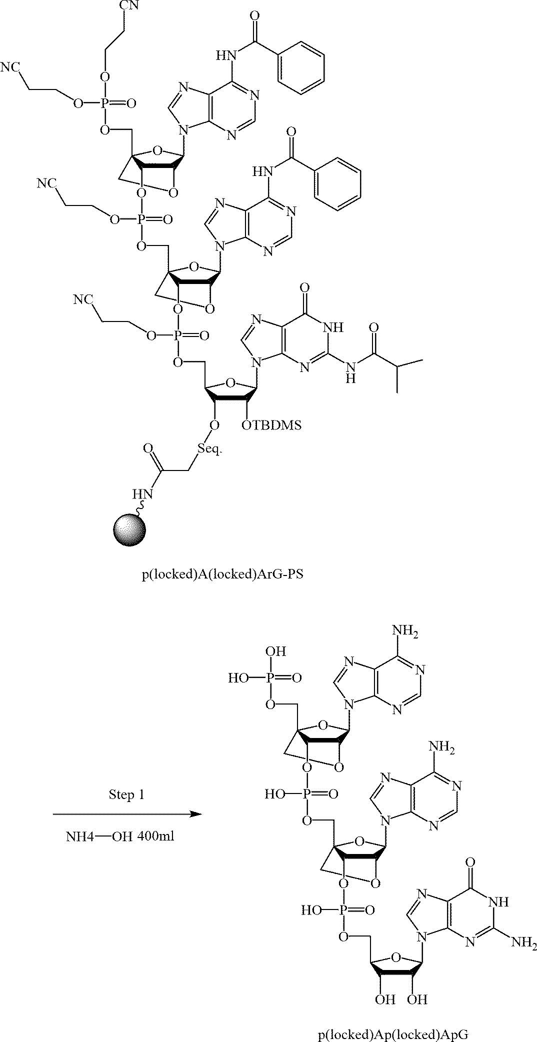

In a 1L bottle, p(locked)Ap(locked)A G-PS (1.60 g from GENERAL Biosystems (Anhui) and Corporation Limited) and 30% ammonia aqueous solution (350 mL) was added together, heated to 40° C. and stirred for 20 h, then cooled to room temperature. The resulting mixture was filtered and washed with water. The crude product was concentrated and diluted with 250 mL water. The aqueous layer was purified on a 150 mL DEAE column. The product fractions were combined, concentrated, and freeze dried as a white solid (405 mg, 388 µmol). MS(m/z): found 1047 [M+H+], 1H NMR (400 MHz, D20): δ8.11 (s, 1H), 7.92(s, 1H), 7.63(s, 1H), 7.62 (s, 1H), 7.38 (s, 1H), 5.86 (s, 1H), 5.62 (d, J = 4.56 Hz, 1H), 5.52 (s, 1H), 5.10 (s, 1H), 4.50(d, J = 5.32 Hz, 1H),4.31-3.99 (m, 13H), 3.14-3.08(q, 19H), 1.20(t, 30H). 31P NMR (162 MHz, D2O) δ0.84(S, 1P), -2.07(S, 1P), -2.15(S, 1P).

Synthesis of 7mGppp(locked)Ap(locked)ApG (GL-Cap 1)

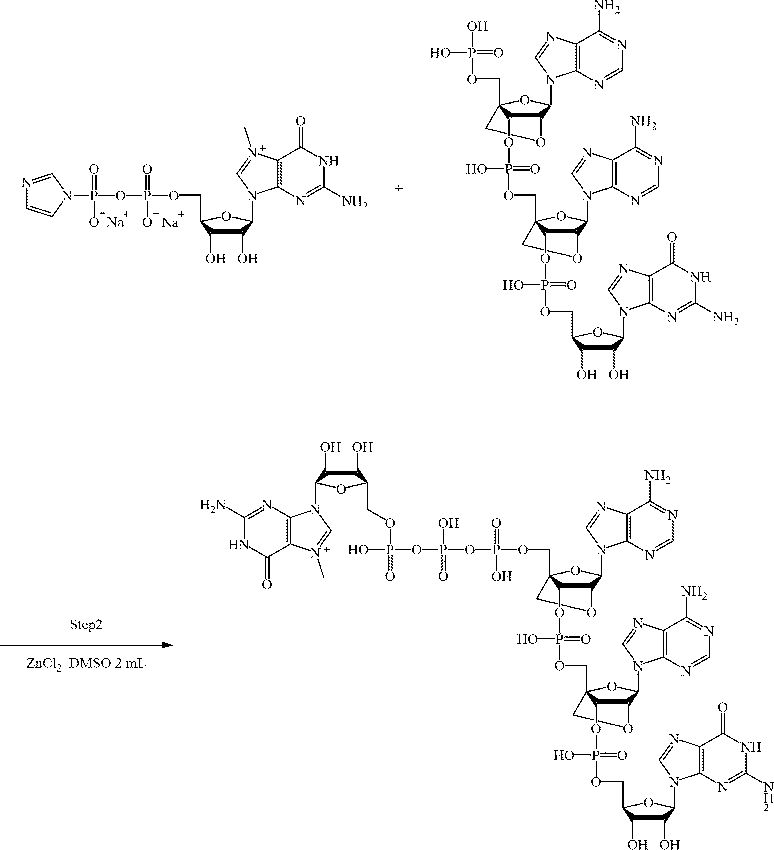

In a 5 mL vial, to a mixture of 7 m-GDP-Imiazole (35.5 mg, 67 µmol), p(locked)Ap(locked)ApG TEA (62.7 mg, 60 µmol) and zinc chloride (240 mg, 1.8 mmol) was added dry DMSO (2 mL), and then the whole mixture was stirred at 35° C. for 20 h. The reaction mixture was added to a solution of EDTA disodium (804 mg, 8.0 mmol) in 100.0 mL of water at 0° C. The resulting aqueous solution was adjusted to pH 6.0 and loaded on a DEAE Sephadex column. The desired product was eluted using a linear gradient of 0-1 M TEAB and the fractions containing the product were pooled, evaporated and concentrated to 27 mg white solid. The crude product was purified on HPLC to yield ammonium salt (16 mg, 10.77 µmol). Yield 18.0%. MS(m/z): found 1486 [M+H+]. 1H NMR (400 MHz, D20): δ9.00(s, 1H), 8.00 (s 1H), 7.96(s, 1H), 7.64 (s, 1H), 7.62 (s, 1H), 7.38 (s, 1H), 5.62 (s,1H), 5.61-5.55 (m, 3H), 5.02(s, 1H), 4.70-3.99 (m, 19H), 3.97(s, 3H), 31P NMR (162 MHz, D20) δ-2.12(s,2P), -11.44(d, 2P), -22.71(t, 1P).

### Example 11

Synthesis of 7mGppp(2′-F)Ap(2′-F)ApG (GL-Cap 2) Synthesis of p(2′-F)Ap(2′-F)ApG

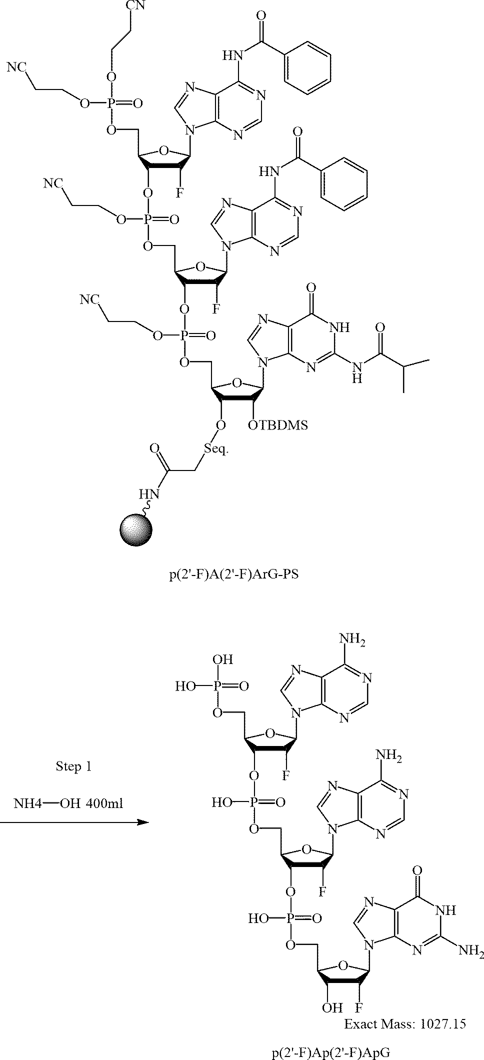

In a 1L bottle, p(2′F)Ap(2′F)A G-PS (1.60 g from GENERAL Biosystems (Anhui) and Corporation Limited) and 30% ammonia aqueous solution (350 mL) were added together, heated to 40° C. and stirred for 20 h, then cooled to room temperature. The resulting mixture was filtered and washed with water. The crude product was concentrated and diluted with 250 mL water. The aqueous layer was purified on a 150 mL DEAE column. The product fractions were combined, concentrated, and freeze dried as a white solid (253 mg, 247 µmol). MS(m/z): found 1027 [M+H+], 1H NMR (400 MHz, D20): δ8.31 (s, 1H), 7.89(s, 2H), 7.66(s, 1H), 7.620(s, 1H), 6.09 (d, J = 14.36 Hz, 1H), 5.99 (d, J = 15.8 Hz, 1H), 5.60 -5.48(m, 2H), 5.26-5.13(m, 1H), 4.61(m,14H), 3.14-3.08(q, 22H), 1.20(t, 32H). 31P NMR (162 MHz, D20): δ1.33(S, 1P), -1.40(s, 1P), -1.76(S,1P). Synthesis of 7mGppp(2′-F)Ap(2′-F)ApG (GL-Cap 2)

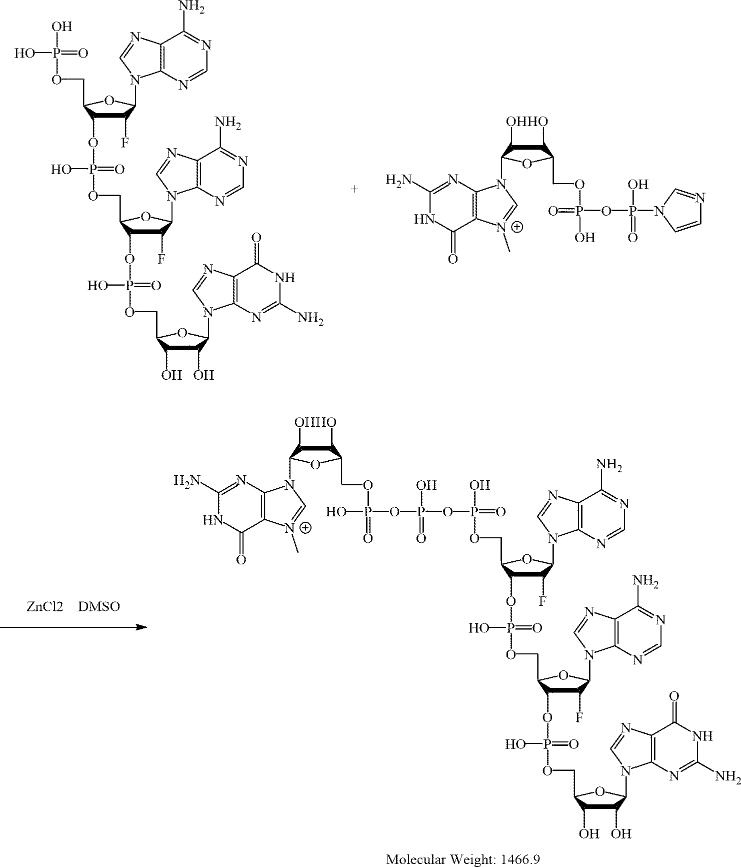

In a 5 mL vial, to a mixture of 7 m-GDP-Imiazole (29.5 mg, 55 µmol), p(2′-F)Ap(2′-F)ApG TEA (50.6 mg, 49.5 µmol) and zinc chloride (200 mg, 1.5 mmol) was added dry DMSO (2 mL), and then the whole mixture was stirred at 35° C. for 20 h. The reaction mixture was added to a solution of EDTA disodium (670 mg, 1.8 mmol) in 100.0 mL of water at 0° C. The resulting aqueous solution was adjusted to pH 6.0 and loaded on a DEAE Sephadex column. The desired product was eluted using a linear gradient of 0-1 M TEAB and the fractions containing the product were pooled, evaporated and concentrated to 26 mg white solid. The crude product was purified on HPLC to yield ammonium salt. (9.3 mg, 6.3 pmol). Yield 12. 80%. MS (m/z): found 1468 [M+H⁺], ¹H NMR (400 MHz, D₂O): δ9.01 (s, 0.59H), 8.27 (s, 1H), 8.02 (s, 1H), 7.99 (s, 1H),7.80 (s, 1H), 7.69 (s, 1H),6.04 (dd, J = 15.6, 2H), 5.83 (d, J = 3.64, 1H), 5.60 (d, J = 5.88, 1H), 5.46-5.29 (m,2H), 4.70-3.97 (m,22H), 31P NMR (162 MHz, D20) δ-1.38 (s,1P), -2.13 (s,1P),-11.26 (d, 2P), -22.58 (t, 1P).

### Example 12

Synthesis of 7mGppp(2′-F)Ap(locked)ApG (GL-Cap 3) Synthesis of p(2′-F)Ap(locked)ApG

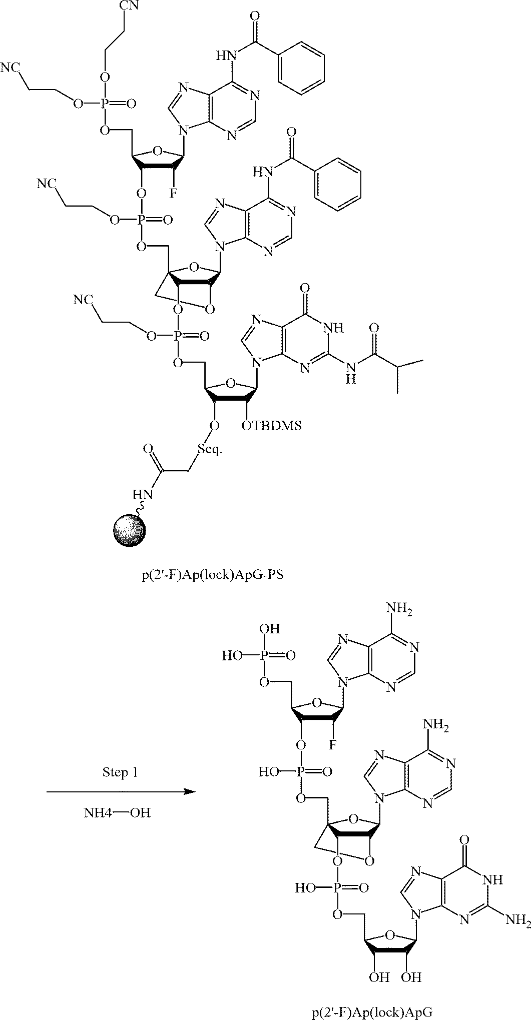

In a 1 L bottle, p(2′F)Ap(locked)ApG-PS (1.60 g from GENERAL Biosystems (Anhui) and Corporation Limited) and 30% ammonia aqueous solution (350 mL) were added together, heated to 40° C. and stirred for 20 h, then cooled to room temperature. The resulting mixture was filtered and washed with water. The crude product was concentrated and diluted with 250 mL water. The aqueous layer was purified on a 150 mL DEAE column. The product fractions were combined, concentrated, and freeze dried as a white solid (236 mg, 228 µmol). MS (m/z): found 1037 [M+H+], 1H NMR (400 MHz, D20): δ8.28 (s, 1H), 7.88 (s, 1H), 7.64 (s, 1H), 7.57 (s, 1H), 6.17 (d,13.8 Hz, 1H), 5.79 (s, 1H), 5.66 (s, 1H), 5.62 (d, J = 4.52 Hz, 1H), 4.61 (d, J = 5.16 Hz, 1H), 4.48 (d, J = 6.52 Hz, 1H), 4.32-4.01 (m,11H), 3. 14-3.08 (q, 17H), 1.20 (t, 26H). 31P NMR (162 MHz, D20): δ 0.17 (s, 1P), -1.30 (S, 1P), -2.09 (s, 1P).

Synthesis of 7mGppp(2′-F)Ap(locked)ApG (GL-Cap 3)

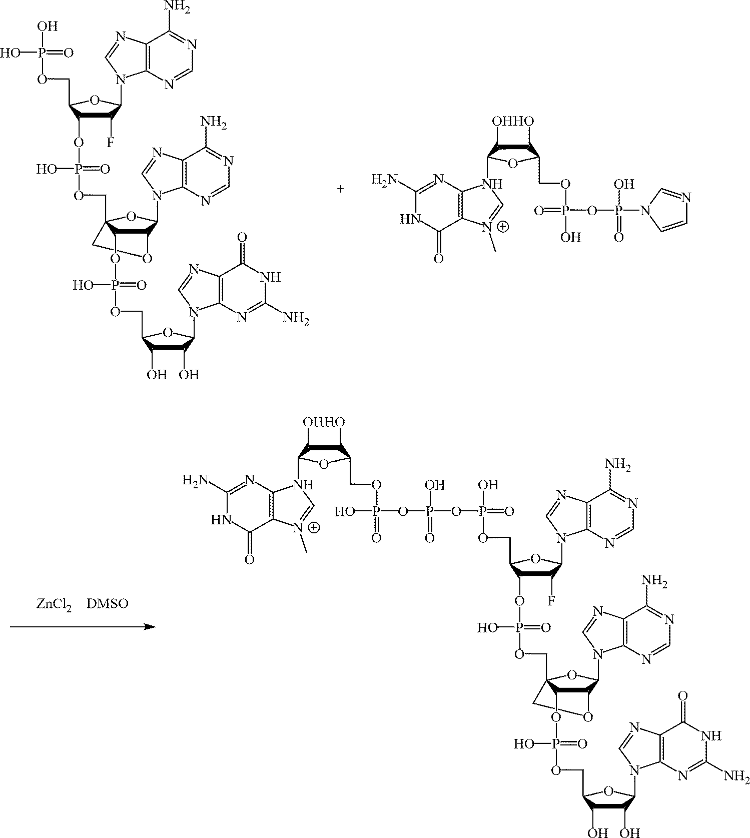

In a 5 mL vial, to a mixture of 7 m-GDP-Imiazole (48.3 mg, 87.5 µmol), p(2′-F)Ap(locked)ApG TEA (72.5 mg, 70 µmol) and zinc chloride (235 mg, 1.75 mmol) was added dry DMSO (2 mL), and then the whole mixture was stirred at 35° C. for 20 h. The reaction mixture was added to a solution of EDTA disodium (782 mg, 2.1 mmol) in 100.0 mL of water at 0° C. The resulting aqueous solution was adjusted to pH 6.0 and loaded on a DEAE Sephadex column. The desired product was eluted using a linear gradient of 0-1 M TEAB and the fractions containing the product were pooled, evaporated and concentrated to 24 mg white solid. The crude product was purified on HPLC to yield ammonium salt (16 mg, 10.8 µmol). Yield 15.4%. MS (m/z): found 1477 [M+H⁺], ¹H NMR (400 MHz, D₂O): δ8.96 (s, 0.7H),8.33 (s 1H), 8.10 (s, 1H), 7.79 (s, 1H), 7.69 (s, 1H), 7.65 (s, 1H), 6.14 (d, J = 13.32 Hz,1H), 5.74-5.68 (m, 3H),5.63 (d, J = 5.68 Hz, 2H), 4.70 (s, 2H), 4.58 (d, J = 5.48 Hz, 1H), 4.50 (d, J = 6.12 Hz, 1H),4.43-4.05 (m, 18H), 3.90 (s, 3H), ³¹P NMR (162 MHz, D₂O) δ-1.28 (S, 1P), -1.97 (S, 1P), -11.38 (d, 1P),-22.61 (t, 1P).

### Example 13

Synthesis of 7mGppp(locked)Ap(2′-F)ApG (GL-Cap 4) Synthesis of p(locked)Ap(2′-F)ApG

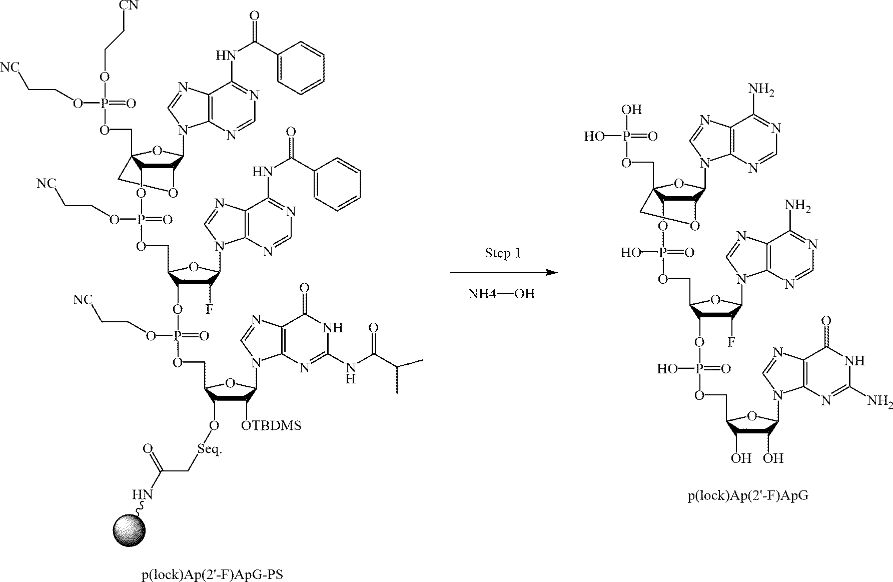

In a 1 L bottle, p(locked)Ap(2′F)ApG-PS (1.60 g from GENERAL Biosystems (Anhui) and Corporation Limited) and 30% ammonia aqueous solution (350 mL) were added together, heated to 40° C. and stirred for 20 h, then cooled to room temperature. The resulting mixture was filtered and washed with water. The crude product was concentrated and diluted with 250 mL water. The aqueous layer was purified on a 150 mL DEAE column. The product fractions were combined, concentrated, and freeze dried as a white solid (265 mg, 256 µmol). MS (m/z): found 1037 [M+H⁺], ¹H NMR (400 MHz, D₂O): δ8.13 (s, 1H), 7.91 (s, 1H), 7.85 (s, 1H), 7.59 (s, 1H), 7.58 (s, 1H), 5.95 (d, 14.96 Hz, 1H), 5.84 (s, 1H), 5.55 (d, J = 6.32 Hz, 1H), 5.20-5.07 (m, 1H), 5.06 (d, J =5.76 Hz, 1H), 4.53 ( t, 5.56 Hz, 1H),4.44-3.99 (m,11H), 3. 14-3.08 (q, 17H), 1.20 (t, 26H). ³¹P NMR (162 MHz, D₂O): δ 0.23 (S,1P),-1.44 (S,1P), -2.55 (S,1P)

Synthesis of 7mGppp(locked)Ap(2′-F)ApG (GL-Cap 4)

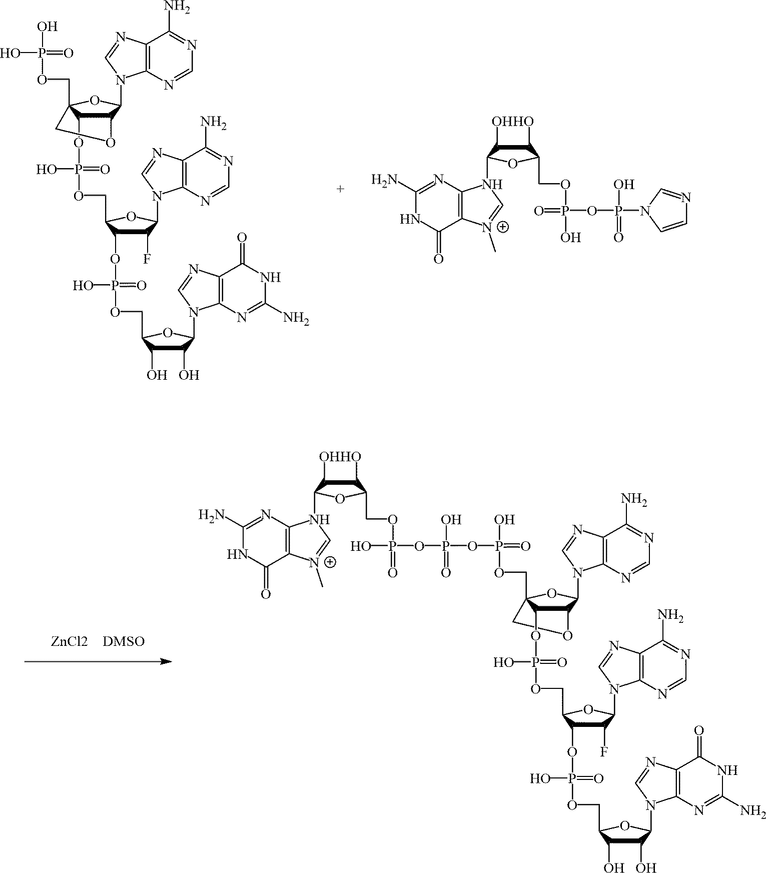

In a 5 mL vial, to a mixture of 7m-GDP-Imiazole (45.5 mg, 82.5 µmol), p(locked)Ap(2′-F)ApG TEA (68.3 mg, 66 µmol) and zinc chloride (221 mg, 1.65 mmol) was added dry DMSO (2 mL), and then the whole mixture was stirred at 35° C. for 20 h. The reaction mixture was added to a solution of EDTA disodium (737 mg, 2.0 mmol) in 100.0 mL of water at 0° C. The resulting aqueous solution was adjusted to pH 6.0 and loaded on a DEAE Sephadex column. The desired product was eluted using a linear gradient of 0-1 M TEAB and the fractions containing the product were pooled, evaporated and concentrated to 34 mg white solid. The crude product was purified on HPLC to yield ammonium salt (23 mg, 15.6 µmol). Yield 23%. MS (m/z): found 1477 [M+H+], 1H NMR (400 MHz, D20): δ 9.03 (s, 0.58H),7.99 (s 1H), 7.90 (s, 1H), 7.81 (s, 1H), 7.56 (s, 1H), 7.55 (s, 1H), 5.93 (d, J = 15.04 Hz 1H), 5.81 (d, J = 4.28 Hz 1H), 5.55 (d, J = 6.32 Hz 1H), 5.52 (s, 1H),5.17-5.04 (m, 1H), 4.92 (s,1H), 4.70-4.01 (m,23H), 31P NMR (162 MHz, D20) δ-1.45 (s, 1P), -2.78 (S, 1P), -11.50 (d, 2P), -22.84 (t, 1P).

### Example 14

Synthesis of 7mGppp(locked)ApG (GL-Cap 5) Synthesis of p(locked)ApG

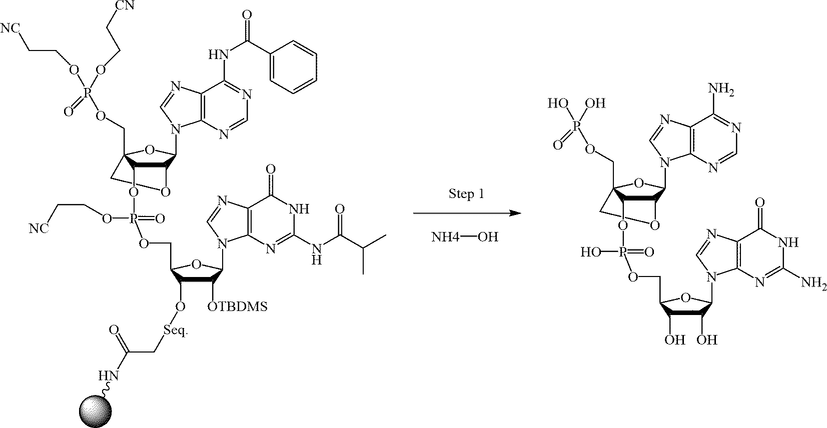

In a 1 L bottle, p(locked)ApG-PS (2.0 g from GENERAL Biosystems (Anhui) and Corporation Limited) and 30% ammonia aqueous solution (350 mL) were added together, heated to 40° C. and stirred for 20 h, then cooled to room temperature. The resulting mixture was filtered and washed with water. The crude product was concentrated and diluted with 250 mL water. The aqueous layer was purified on a 150 mL DEAE column. The product fractions were combined, concentrated, and freeze dried as a white solid TEA salt (193 mg, 274 µmol).

MS (m/z): found 705 [M+H+], 1H NMR (400 MHz, D20): δ8.15 (s, 1H), 7.92 (s, 1H), 7.71 (s, 1H), 5.92 (s, 1H), 5.63 (d, J = 4.4 Hz, 1H), 4.95 (s, 1H), 4.33-4.05 (m, 9H), 3. 14-3.00 (q, 16H), 1.20 (t, 24H). 31P NMR (162 MHz, D20) δ1.93 (s, 1P), 2.29 (S, 1P).

Synthesis of 7mGppp(locked)ApG (GL-Cap 5)

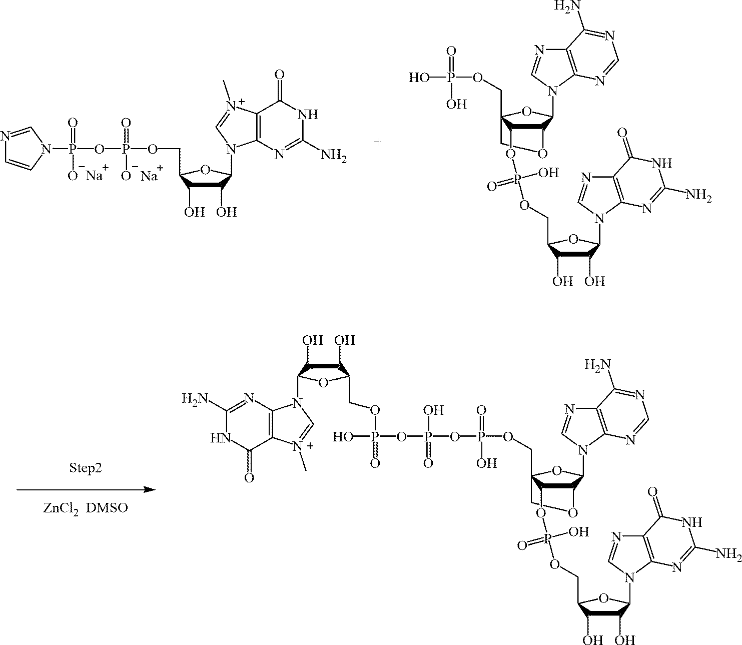

In a 5 mL vial, to a mixture of 7 m-GDP-Imiazole (140 mg, 228 µmol), p(locked)ApG TEA (160 mg, 227 µmol) and zinc chloride (915 mg, 6.84 mmol) was added dry DMSO (2 mL), and then the whole mixture was stirred at 35° C. for 20 h. The reaction mixture was added to a solution of EDTA disodium (3.0 g, 8.0 mmol) in 100.0 mL of water at 0° C. The resulting aqueous solution was adjusted to pH 6.0 and loaded on a DEAE Sephadex column. The desired product was eluted using a linear gradient of 0-1 M TEAB and the fractions containing the product were pooled, evaporated and concentrated to 106 mg white solid. The crude product was purified on HPLC to yield ammonium salt (48.3 mg, 42 µmol). Yield 18.5%. MS (m/z): found 1144 [M+H+], 1H NMR (400 MHz, D20): δ9.05 (s, 1H), 8.11 (s, 1H), 8.01 (s, 1H), 7.76 (s, 1H), 5.80 (d, J = 4.1 Hz, 1H), 5.67 (s, 1H), 5.62 (d, J = 4.4 Hz 1H), 4.85 (s, 1H), 4.70 (m, 2H), 4.51-4.02 (m, 13H), 4.00 (s, 3H). 31P NMR (162 MHz, D20) δ-1.96 (S, 1P), -11.53 (d, 2P), -22.95 (t, 1P).

### Example 15

Synthesis of 7m-3′-MOEGppp(locked)ApG (GL-Cap 6)

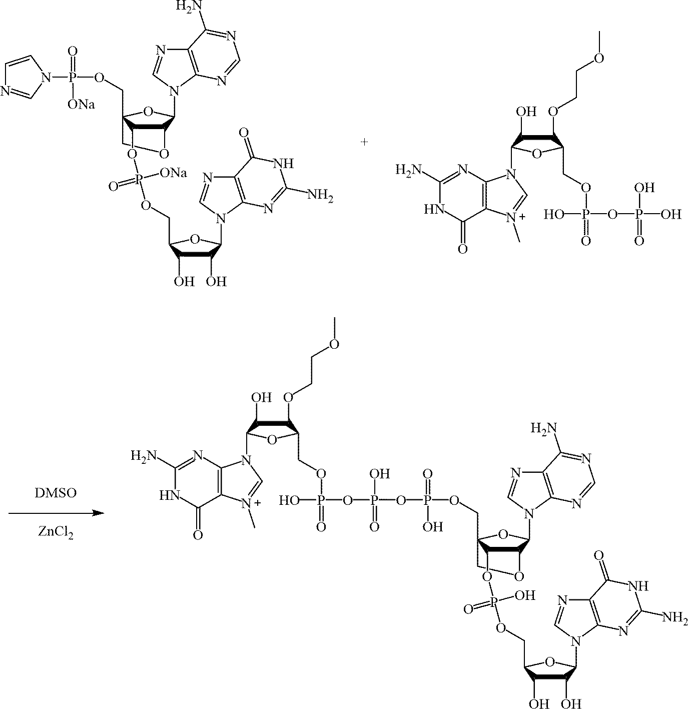

In a 5 mL vial, to a mixture of 7m-2′-MOE-GDP TEA (20.7 mg, 40.1 µmol), p(locked)ApG-Imiazole (32.0 mg, 40.1 µmol) and zinc chloride (164 mg, 1.2 mmol) was added dry DMSO (0.8 mL), and then the whole mixture was stirred at 35° C. for 20 h. The reaction mixture was added to a solution of EDTA disodium (580 mg, 1.6 mmol) in 100.0 mL of water at 0° C. The resulting aqueous solution was adjusted to pH 6.0 and loaded on a DEAE Sephadex column. The desired product was eluted using a linear gradient of 0-1 M TEAB and the fractions containing the product were pooled, evaporated and concentrated to crude product. The crude product was purified on HPLC to yield ammonium salt (7.5 mg, 6 µmol). Yield 15 %. MS (m/z): found 1203 [M+H+], 1H NMR (400 MHz, D20): δ 8.99 (s, 1H),8.37 (s 1H), 7.90 (s, 1H), 7.87 (s, 1H), 7.66 (s, 1H), 5.78 (d, 1H), 5.60 (m, 2H), 4.80 (s, 1H), 4.54-4.14 (m, 17H), 4.01 (S, 3H), 3.7 (m, 2H), 3.57 (m, 2H), 3.32 (s, 3H), 31P NMR (162 MHz, D20) δ-2.02 (s, 1P), -11.57 (d, 2P), -22.77 (m, 1P)

### Example 16

Synthesis of 7m-2′F-Gppp(locked)ApG (GL-Cap 7)

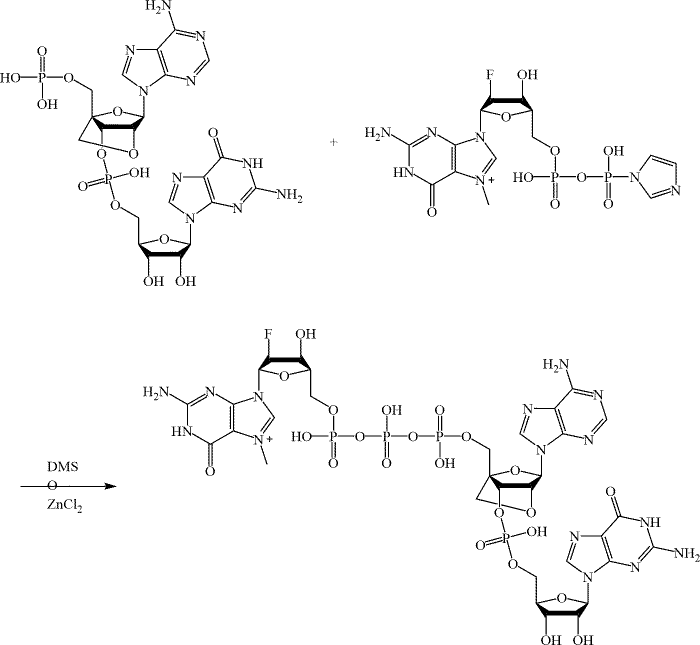

In a 5 mL vial, to a mixture of 7m-2′-F-GDP-Imiazole (35 mg, 66 µmol), p(locked)ApG (60.0 mg, 85 µmol) and zinc chloride (228 mg, 1.7 mmol) was added dry DMSO (1.0 mL), and then the whole mixture was stirred at 35° C. for 20 h. The reaction mixture was added to a solution of EDTA disodium (830 mg, 2.1 mmol) in 100.0 mL of water at 0° C. The resulting aqueous solution was adjusted to pH 6.0 and loaded on a DEAE Sephadex column. The desired product was eluted using a linear gradient of 0-1 M TEAB and the fractions containing the product were pooled, evaporated and concentrated to crude product. The crude product was purified on HPLC to yield ammonium salt (5 µmol). Yield 6 %. MS (m/z): found 1147 [M+H+], 1H NMR (400 MHz, D20): 8.03 (s 1H), 7.89 (s, 1H), 7.68 (s, 1H), 6.09 (d, 1H), 5.68 (s, 1H), 5.61 (d, 1H), 5.32-5.19 (m, 1H), 4.85 (s, 1H), 4.57-4.00 (m, 17H), 31P NMR (162 MHz, D20) δ-2.04 (s, 1P), -11.51 (D,2P), -22.73 (t, 1P)

### Example 17

Synthesis of 7m-2-′MOE-Gppp(locked)ApG (GL-Cap 8)

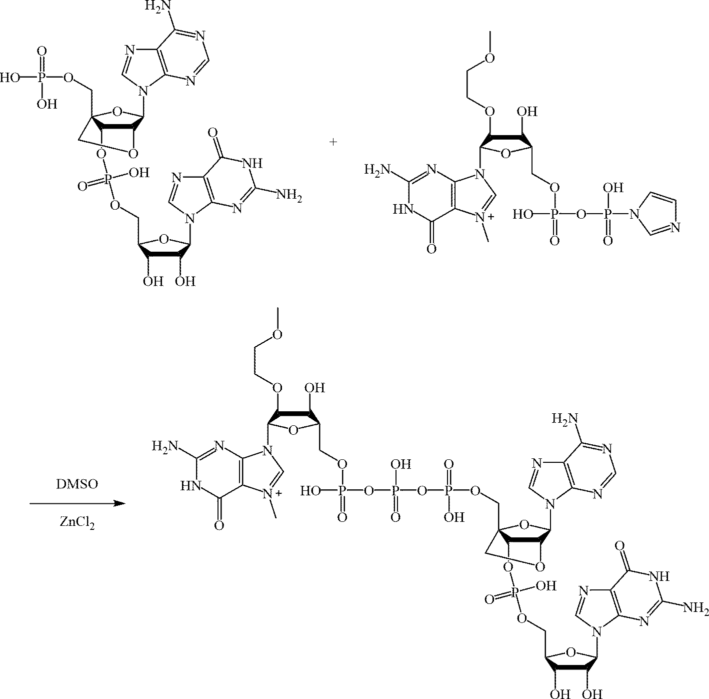

In a 5 mL vial, to a mixture of 7m-2′-MOE-GDP-Imidazole (43 mg, 76 µmol), p(locked)ApG (55 mg, 76 µmol) and zinc chloride (215 mg, 1.5 mmol) was added dry DMSO (1 mL), and then the whole mixture was stirred at 35° C. for 40 h. The reaction mixture was added to a solution of EDTA disodium (860 mg, 2.2 mmol) in 100.0 mL of water at 0° C. The resulting aqueous solution was adjusted to pH 6.0 and loaded on a DEAE Sephadex column. The desired product was eluted using a linear gradient of 0-1 M TEAB and the fractions containing the product were pooled, evaporated and concentrated to crude product. The crude product was purified on HPLC to yield ammonium salt (4 µmol). Yield 5 %. MS (m/z): found 1203 [M+H+], 1H NMR (400 MHz, D20): δ 9.02 (S, 1H), 8.16 (s, 1H),8.04 (s 1H), 7.80 (s, 1H), 5.89 (d, 1H), 5.76 (s, 1H), 5.65 (d, 1H), 4.86 (s, 1H), 4.70-4.01 (m, 12H), 4.61-4.57 (m, 2H), 4.41-3.96 (m,14H), 31P NMR (162 MHz, D20) δ-1.32 (S, 1P), -11.50 (d, 2P), -22.74 (t, 1P).

### Example 18

Synthesis of 7m-3′-MOE-5′-RmGppp(2′-Ome)ApG (GL-Cap 9)

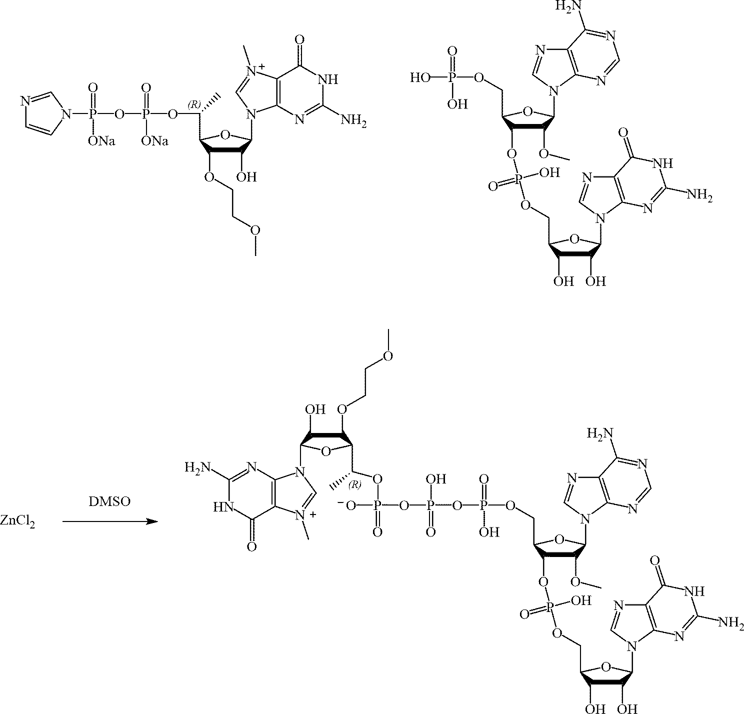

In a 4 mL vial, to a mixture of 7 m-3′-MOE-5′-RmGDP-Im (25 µmol), pAmG-TEA (20 µmol) and zinc chloride (67 mg, 500 µmol) was added dry DMSO (500 µL) and the whole mixture was stirred at 35° C. for 24 h. HPLC detected ∼40% conversion so stirred for an additional 24 h. The whole mixture was diluted with water (50 mL), treated with bound EDTA quadrapure resin (0.5 g), concentrated and purified by prep-HPLC. Fractions in the major peak were combined and freeze dried twice to yield 4.0 µmol product, as ammonium salt. MS(m/z): found 1219 [M+H+], ¹H NMR (400 MHz, D₂O): δ9.27 (S, 1H), 8.41 (s 1H), 8.19 (s, 1H), 7.96 (s, 1H), 6.05 (d, 1H), 5.92 (d, 1H), 5.85 (d, 1H), 4.70-3.97 (m, 14H), 4.08 (S, 3H), 3.44 (S, 3H), 3.30 (S, 3H), 1.29 (d, 3H), ³¹P NMR (162 MHz, D₂O) δ-1.12 (S, 1P), -11.30 (d, 2P), -21.31 (t, 1P).

### EXAMPLE ACTIVITY ASSESSMENT

### Experimental Example 1

Methods for evaluating in vitro activity of 5′-cap structures synthesized Novel 5′-cap structures that were synthesized were incorporated in mRNA via IVT and then assessed for translation efficacy in HeLa cells following transfection. The novel 5′-caps synthesized were incorporated into Gaussia luciferase (Gluc) mRNA using pGL-T7-hGLuc-30+70A (Kana-GGG-SAPI Plasmid Size:3704). Then, the dose responses of the new cap analogs were compared to commercially available CapO from NEB (m7G (5′) ppp(5′) G, cat no. S1404).

Briefly, 1 µg GLuc (GGG plasmid) template per reaction was transcribed in vitro using a High Yield T7 mRNA synthesis kit. CapO (NEB or other analogs) was added by co-transcription. Transcription was carried out for 2 hours, followed by DNase I treatment for 15 min. Ratio of GTP to Cap analogs, 1:4 (2 mM GTP, 8 mM cap). Expected mRNA length: 883/884 nt. Then the IVT mRNAs were purified using Monarch cleanup kits after the DNase step, the RNA was quantitated using Nanodrop and samples were run on a 2100 bioanalyzer. About 500 ng of mRNA was also run on 1% agarose E-Gel (final volume 20 µl) for checking mRNA integrity.

Samples (dose response from 0.03 to 2 µg/ml) were transfected in triplicate in HeLa cells (10,000 cells/0.1 ml/96 well plate) using 2 µl/ml lipofectamine messengerMax to monitor functional (translation). Activity of GLuc mRNA into which different cap analogs have been incorporated for 24 hours. Uncapped sample was used as a control. Gluc protein levels were monitored in the culture supernatants (20 µl supernatant + 50 µl substrate for 10 minutes).

Data for representative compounds are shown in FIGS. 1-6. These data suggest that compounds 2, 6, and 26 have greater activity than cap0 and similar activity as ARCA, while 11, 12, 13, 27, and 50 have comparable or slightly lower activity than cap0. The permutations and combinations of head and tail nucleoside groups and triphosphate linker modifications in this disclosure would permit novel and potent 5′-cap analogs.

### Experimental Example 2

### Preparation of mRNA by Incorporating Novel 5′-Caps Synthesized

The novel 5′ -caps synthesized were incorporated into Firefly luciferase (Fluc) mRNA using pGL-T7-AG-nUTR002-Fluc-HBB3UTR-30+70A-BpiI (Kana-AGG-BpiI Plasmid Size: 4856bp). Then, the dose responses of the new cap analogs were compared to commercially available Cap analog from Trilink m7G(5′)ppp(5′)(2′OMeA)pG (cat no. N-7113).

Briefly, 1 µg GLuc (AGG plasmid) template per reaction was transcribed in vitro using a High Yield T7 mRNA synthesis kit (NEB, E2040S). Cap analogs were added by co-transcription. Transcription was carried out for 2 hours, followed by DNase I treatment for 15 min. Ratio of NTP to Cap analogs, 10:4 (10 mM NTP each, 4 mM cap).

Expected mRNA length: 2008 nt. Then the small scale IVT mRNAs were purified using DNA Selection Magnetic beads (Yeasen,12601ES56), Large scale IVT mRNAs were purified using POROS™ Oligo (dT)25 (Thermofisher, A47383) and Ultrafiltration use Amicon® Ultra-15(Merk, UFC910024), the RNA was quantitated using Nanodrop, and 500 ng of each mRNA was run on agarose E-Gel, mRNAs were run on a 2100 bioanalyzer or CE (PA800) for checking mRNA integrity, HPLC for Purity, DNAzyme-PAGE for Capping efficiency and polyA quality. Large scale mRNA were subjected to more QC (Capping/polyA tail-LC-MS, Template DNA residual-qPCR, dsRNA-Elisa, pH).

### Experimental Example 3

### In Vitro Evaluation of the Synthesized 5′-cap Structures

Novel 5′-cap structures that were synthesized were incorporated into GL2288 mRNA via IVT and then assessed for translation efficacy in HeLa cells following transfection. Briefly, HeLa cells were seeded in a 96-well plate (150000cells/0.1 ml/well). The next day (approx. -80-90% confluent), the cells were transfected with 100, 25, 6.25 ng/well of GL2288 mRNA samples using 2 µl/ml of Lipofectamine MessengerMax (Thermo Scientific, cat#LMRNA015) or in-house formulated lipid-nanoparticles (LNPs). The cells were incubated for another 24 h. To monitor the functional (translation) activity of GL2288 mRNAs into which different 5′cap analogs have been incorporated, the protein expression was evaluated by intracellular staining using fluorophore-conjugated antibody specific to the protein expressed by GL2288 mRNA. All assays were run in triplicates. Statistical analysis was performed using two-way ANOVA followed by uncorrected Fisher’s LSD multi-comparison test.

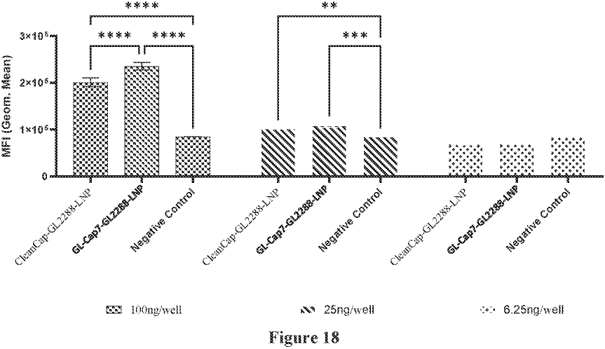

Representative results are shown in FIGS. 17 and 18. As shown in FIG. 17, CleanCap- and GL-Cap7-GL2288 mRNAs showed similar translation efficiency as shown by the total cells expression the target protein (a); and by the proteins expression levels registered as mean fluorescence intensity (b). As shown in FIG. 18, at a high dose, the GL-Cap7-GL2288 mRNA showed a significantly higher translation efficiency than to CleanCap-GL2288 as evaluated by the total protein expression levels. No difference was registered at medium and low doses between the two mRNAs.

### Experimental Example 4

### Evaluation of the Synthesized 5′-Cap Structures

### A. In Vitro Evaluation of the Synthesized 5′-Cap Structures

Novel 5 ′-cap structures that were synthesized were incorporated into Firefly luciferase (Fluc) mRNA via IVT as described above and then assessed for translation efficacy in HeLa cells following transfection. Briefly, HeLa cells were seeded in a 96-well plate (20000cells/0.1 ml/well). The next day (approx. ∼80-90% confluent), the cells were transfected with 25, 50, or 100 ng/well of Fluc-mRNA samples using 2 µl/ml of Lipofectamine MessengerMax (Thermo Scientific, cat#LMRNA015) or in-house formulated lipid-nanoparticles (LNPs). The cells were incubated for another 24 h. To monitor the functional (translation) activity of Fluc mRNAs into which different 5′cap analogs have been incorporated, the Fluc protein expression was evaluated by adding 60 µl Bio Lite Luciferase Assay System (Vazyme, cat # DD1201-02) into each well, incubating the plates at room temperature for 10 min, and measuring the luminescence using a multimode plate reader luminometer. All wells were assayed in triplicates. Statistical analysis was performed using two-way ANOVA followed by uncorrected Fisher’s LSD.

### B. In Vivo Evaluation of the Synthesized 5′-Cap Structures

Female C57BL/6 mice 7-8 weeks of age were randomly allocated to experimental groups, 30 mice per group. For each group, 6 experimental conditions were set up: 2 mRNA doses (0.01, 0.1 mg/kg) and 3 time points 6 h, 24 h, 48 h (5 mice/dose/time point). A single intramuscular injection of mRNA-loaded LNPs was performed (injection volume 40 µl). At 6 h, 24 h and 48 h post-injection, 5 animals/dose in each group were taken for in vivo tissue and organ imaging. The animals were shaved and intraperitoneally injected with 200 µL of a d-luciferin solution (15 mg/mL in DPBS) D-luciferin and anesthetized 8 min later using isoflurane (5% induction, 2% maintenance in pure oxygen). The emitted light was measured by in vivo imaging system (IVIS) exactly 12 min after d-luciferin injection. Whole body supine and lateral position imaging was carried out.

After Whole body imaging, the animals were euthanized, and the leg muscles (injection site) were harvested in Experiment 1; muscles and livers were harvested in Experiment 2. The emitted light by the tissues/organs was measured by IVIS in less than 25 min after d-luciferin injection.

Next, tissue lysates were prepared by homogenization and Glo Lysis Buffer 1X (Abcam cat # ab6789) treatment. The lysates were centrifuged at 4° C. for 15 min at 10000 g, and the supernatants were used for total protein quantification and luciferase activity assessment. Total protein concertation was determined using Pierce BCA Protein Assay Kit (thermo Cat # 23227). Then, 50µl of supernatant was mixed with Bio Lite Luciferase Assay System (Vazyme DD1201-02), and the luminescence was measured by a multimode plate reader luminometer.

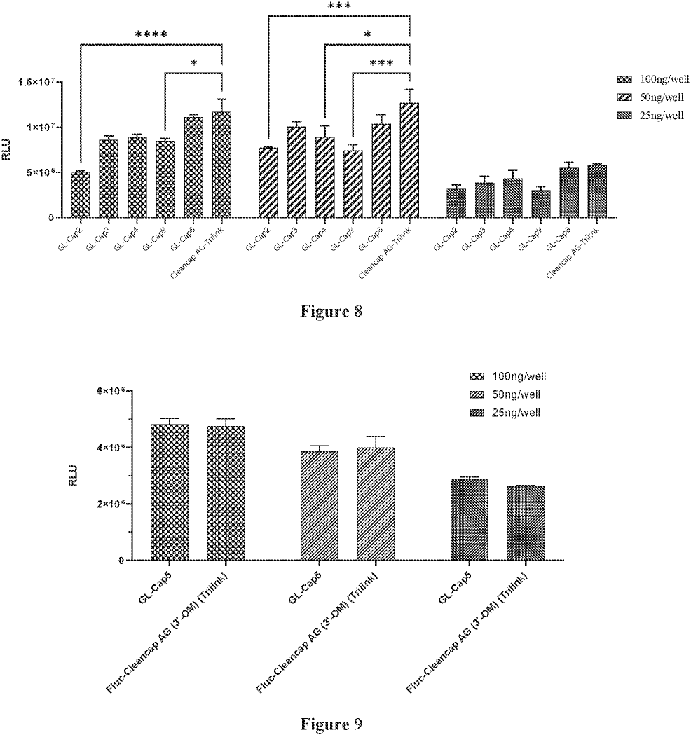

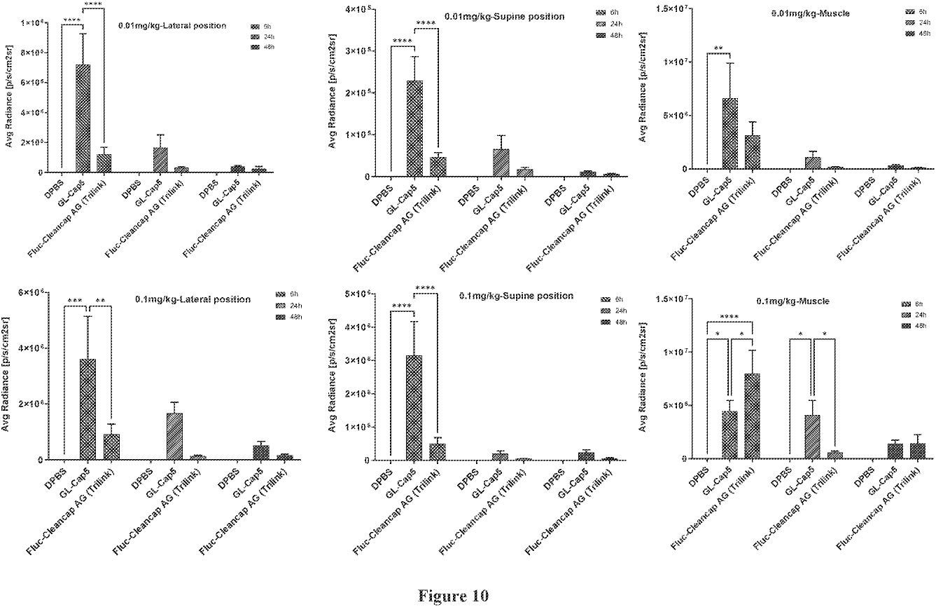

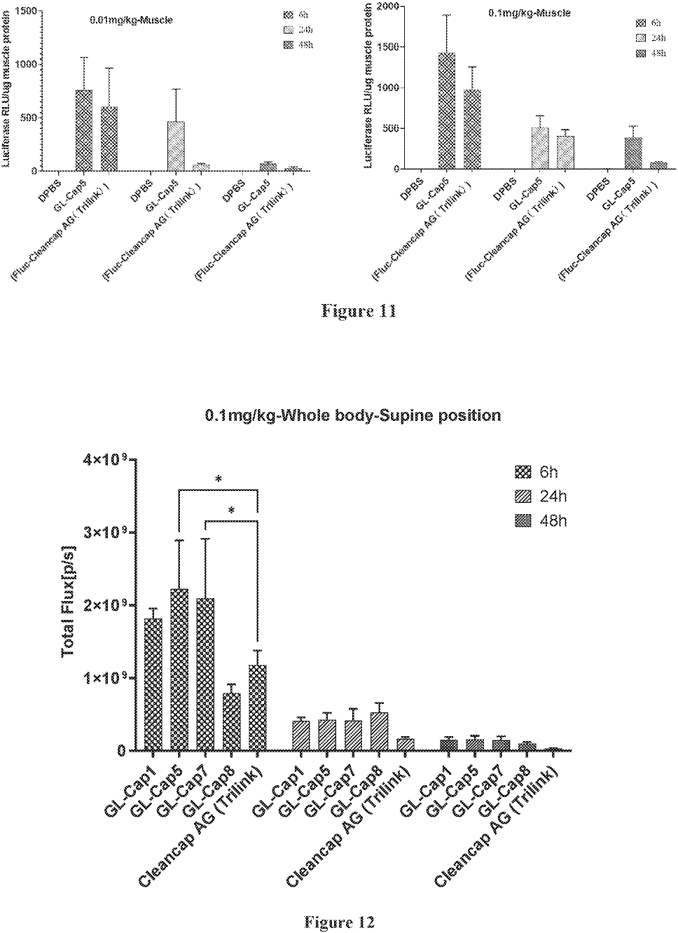

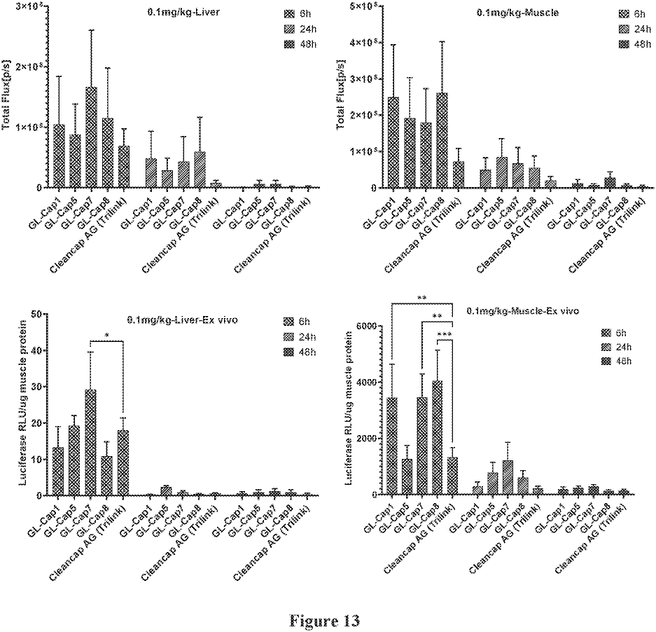

Data for representative cap analogs are shown in FIGS. 7-13. These data suggest that in vitro, as shown in FIGS. 7-9, compared to the control Cleancap AG, caps GL-Cap5 and GL-Cap7 exhibited a stronger expression, while GL-Cap1, GL-Cap6, and GL-Cap8 showed a similar to slightly stronger expression, and GL-Cap2, GL-Cap3, GL-Cap4 and GL-Cap9 demonstrated a slightly weaker expression; when compared to Cleancap AG in vivo, as shown in FIGS. 10-13, GL-Cap5 and GL-Cap7 maintained a significantly stronger expression, while GL-Cap1 and GL-Cap8 showed a similar expression.

### Experimental Example 5

### Evaluation of the Synthesized 5′-Cap Structures on C3 Tumor Model

As soon as the C3 tumors reached 100 mm³, our HPV E6/E7 mRNA sequence (GL2281) (wherein the sequence encoding HPV E6-E7 is SEQ ID NO:2), capped with either CleanCap, GL-Cap5 or GL-Cap1, was administered intramuscularly at 10 µg per dose, with the first dose on Day 0 and second dose on Day 10. The PBS and 10 µg of CleanCap-capped Luciferase mRNA (FF luciferase) were used for comparison.

As soon as the C3 tumors reached 100 mm3, our HPV E6/E7 mRNA sequence (GL2288) (wherein the sequence encoding HPV E6-E7 is SEQ ID NO:1), capped with either CleanCap (CC), or GL-Cap7, was administered intramuscularly at different doses at Day 0. The CleanCap-capped Luciferase mRNA (FF Luciferase) was used for comparison.

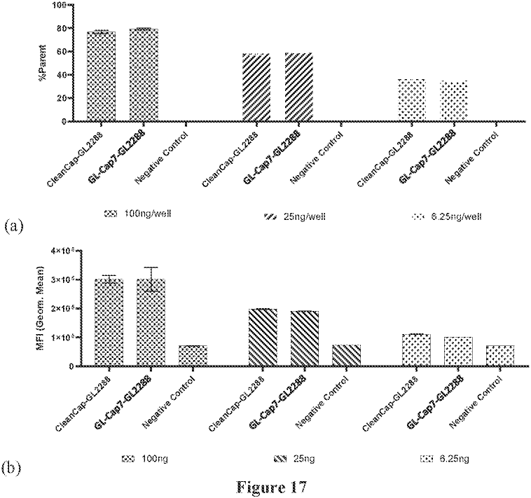

As shown in FIG. 14, all three capped GL2281 mRNAs significantly inhibited tumor growth (Data presented as mean±SEM).

As shown in FIG. 15, all capped GL2288 mRNAs significantly inhibited tumor growth (Data presented as mean±SEM), but at mid dose (0.05 mg/Kg), the GL-Cap7 capped mRNA showed higher anti-tumor efficacy than CC.

### Experimental Example 6

### Evaluation of the Synthesized 5′-Cap Structures on MC38 Tumor Model

As soon as the MC38 tumors reached 100 mm³, our GL-Cap9 capped IL12 mRNA (GL-001) was administered intratumorally once and at different doses, ranging from 0.3 µg to 10 µg. The highest dose (10 µg) of CleanCap-capped Luciferase mRNA and CleanCap-capped Moderna IL12 mRNA (MD-001) were used for comparison.

As shown in FIGS. 16A and 16B, all doses of GL-001 inhibited tumor growth and improved survival better than MD-001. In particular, the lowest dose (0.3 µg) of GL-001 was potent enough to keep the mean tumor volume (FIG. 16A) under 250 mm³ and the survival at 70 % by day 30. By day 32, all mice dosed with 10 µg of GL-001 were alive, in contrast with the 30% survival recorded in the group dosed with 10 µg of MD-001 (In FIG. 16A, data is presented as mean±SEM, and in FIG. 16B, data is presented as Kaplan-Meier curves).

### Experimental Example 7

### In Vivo Evaluation of the Synthesized 5′-Cap Structures: Immunogenicity of the GL2288

### mRNA Vaccine

Female C57BL/6 mice 7-8 weeks of age were randomly allocated to experimental groups, 5 mice per group. All the animals were vaccinated intramuscularly at d0 and d10 with: 50 µl of PBS (as a control group), CleanCap-GL2288 (3 µg or 10 µg), or GL-Cap7-GL2288 (3 µg or 10 µg). Two LNP formulation were used, hereafter referred to as Formulation 1 and Formulation 2. The different groups were designated as follows: CleanCap-GL2288-3µg-Formulation 1, CleanCap-GL2288-10µg-Formulation 1, GL-Cap7-GL2288-3µg-Formulation 1, GL-Cap7-GL2288-10µg-Formulation 1, CleanCap-GL2288-3 µg-Formulation 2, CleanCap-GL2288-10µg-Formulation 2, GL-Cap7-GL2288-3µg-Formulation 2, GL-Cap7-GL2288-10µg-Formulation 2.

Ten days after the second injection, the animals were sacrificed and the spleens collected. Following standard protocols, single cell suspensions were prepared from the spleens and used to perform and INFy enzyme-linked immunospot (ELISpot) assay. Briefly, 100000 splenocytes were seeded into ELISpot 96-well plates pre-coated with anti-mouse INFy antibody and incubated at 37C overnight with no stimulation or stimulation with a peptide library spanning the whole protein expressed by the GL2288 mRNA (15aa mers with 11 aa overlap). The next day, after cell removal and washing of the plate wells, biotinylated cytokine-specific detection antibody was added to the wells for 2 h. Next, the wells were washed and a streptavidin-enzyme conjugate was added to enable the formation of spots. Finally, a colorimetric substrate was added, and the formed spot and the enzymatic catalysis of the substrate were counted in an automated ELISpot reader. The results were represented as Spot Forming Units (SFU) per a million splenocytes.

As shown in FIG. 19, the results indicated that GL-Cap7-GL2288 mRNA induced a stronger immune response than CleanCap-GL2288 mRNA with both formulation and in both regimens (3 µg and 10 µg), as assessed by INFy ELISpot assay (a), after ex vivo specific stimulation of the mice splenocytes; and (b) Representative wells from the ELISpot assay.

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

### SEQUENCE LISTING

SEQ ID NO:1
atggactgga cctggatcct gttcctggtg gccgccgcca ccagagtgca cagcttccag 60
gacccccagg agagcggcag aaagctgccc cagctgtgca ccgagctgca gaccaccato 120
cacgacatca tcctggagtg cgtgtactgc aagcagcagc tgctgagaag agaggtgtac 180
gacagagacc tgtgcatcgt gtacagagac ggcaacccct acgccgtgtg cgacaagtgc 240
ctgaagttct acagcaagat cagcgagtac agacactact gctacagcct gtacggcacc 300
accctggagc agcagtacaa caagcccctg tgcgacctgc tgatcagatg catcaactgc 360
cagaagcccc tgcagagaca cctggacaag aagcagagat tccacaacat cagaggcage 420
tggaccggca gatgcatgag ctgctgcaga agcagcagaa ccagaagaga gacccagctg 480
agaggcagas agagaagaag ccacggcgac acccccaccc tgcacgagta catgctggac 540
ctgcagcccg agaccaccga gccgagcccg acagagcccc ctacaacatc cagcgaggag 600
gaggacgaga tcgacggooo cgccggooag gocgagcccg acagagccca ctacaacatc 660
gtgaccttct gctgcaagtg cgacagcacc ctgagactft gcgtgcagag cacccacgtg 720
gacataagaa ccctggagga cctgctgatg ggcaccctgg gcatcgtgtg ccccatotgc 780
agccagaagc cc 792

SEQ ID NO:2
atggactgga cctggatcct gttcctggtg gocgccgcca cacgggtgca cagcttccag 60
gacccccagg agaocggcag aaagotgcct cagctgtgta ccgagctgca gaccaccatc 120
cacgacatca tcctggagtg tgtgtactgt aagcagcagc tgctgaggag agaggtgtac 180
gaccgggact agtgtatcgt gtacagggac ggcaatccct acgccgtgtg tgacaagtgc 240
ctgaagttct acagcaagat cagcgagfac cggoactact gcfacsgcct gtacggcacc 300
ctgaagttct acagcaagat cagcgagfac cgocactact gcfacagcct tatcaactgo 360
cagaagcccc tgcagagaca cctggacaag aagcagcggt tccacaacat caggggcaga 420
accctggagc gatgtatgag ccgotgccgg agcagcagaa ccagaaggga gacccagctg 480
agaggccgga agagaagaag ccacggcgat acccccaccc tgcacgagta catgotggac 540
ctgcagcctg agaccacoga totgtacggc tacggccagc tgaatgacag cagcgaggag 600
gaggatgaga togacggccc tgccggccag gccgagcccg acagagccca ctacaacato 660
gtgacctftf gctgtaagtg tgacagcacc ctgagactgt gcgfgcagag cacccacgtg 720
gacatcagaa ccctggagga totfotgatg ggcaccctgg gcatcgtgtg tcccatcfgc 780
tcccagaasc cc 792

1. A compound of formula (II) below or a stereoisomer, tautomer, deuterate or salt thereof: 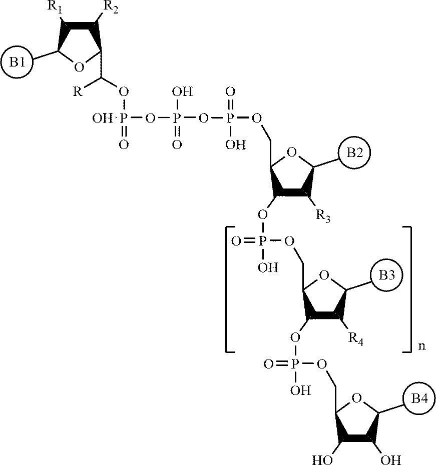
wherein:
ring B1, B2, B3 and B4 are each independently selected from a nucleobase and a modified nucleobase;
each R is independently selected from H and C₁₋₆ alkyl;
R₁ and R₂ are each independently selected from OR₅ and halogen;
each R₃ is independently selected from C₁₋₆ alkoxyl, halogen and LNA;
each R₄ is independently selected from halogen and LNA;
each R₅ is independently selected from H, C₁₋₆ alkyl, C₂₋₆ alkenyl and C₂₋₆ alkynyl, in which C₁₋₆ alkyl, C₂₋₆ alkenyl and C₂₋₆ alkynyl is optionally substituted with one or more of halogen, OH, and/or C₁₋₆ alkoxyl; and
n is an integer selected from 0-1.
.

2. The compound, stereoisomer, tautomer, deuterate or salt of claim 1, wherein ring B1 and B4 are each independently guanine (G), and ring B2 and B3 are each independently adenine (A).

3. The compound, stereoisomer, tautomer, deuterate or salt of claim 1, wherein R is independently selected from H and methyl.

4. The compound, stereoisomer, tautomer, deuterate or salt of claim 1, wherein R₁ and R₂ are each independently selected from OR₅, F, Cl, Br and I, and each R₅ is independently selected from H and C₁₋₆ alkyl, in which C₁₋₆ alkyl is optionally substituted with C₁₋₆ alkoxyl.

5. The compound, stereoisomer, tautomer, deuterate or salt of claim 1, wherein R₁ and R₂ are each independently selected from OH, -O(CH₂)₂OCH₃ and F.

6. The compound, stereoisomer, tautomer, deuterate or salt of claim 1, wherein R₃ is independently selected from methoxy, F and LNA.

7. The compound, stereoisomer, tautomer, deuterate or salt of claim 1, wherein R₄ is independently selected from F and LNA.

8. A compound of formula (IIA) below or a stereoisomer, tautomer, deuterate or salt thereof: 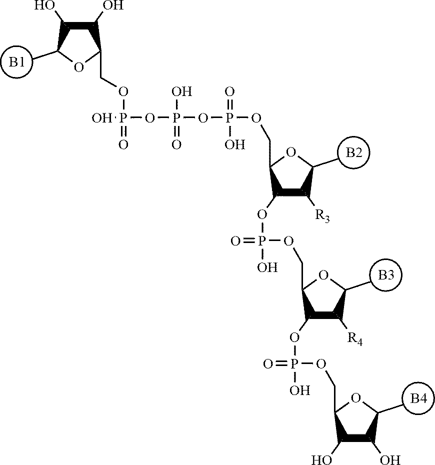
wherein:
ring B1, B2, B3 and B4 are each independently selected from a nucleobase and a modified nucleobase; and
R₃ and R₄ are each independently selected from halogen and LNA.

9. The compound, stereoisomer, tautomer, deuterate or salt of claim 8, wherein ring B1 and B4 are each independently guanine (G), and ring B2 and B3 are each independently adenine (A).

10. The compound, stereoisomer, tautomer, deuterate or salt of claim 8, wherein R₃ and R₄ are each independently selected from F and LNA.

11. A compound of formula (IIB) below or a stereoisomer, tautomer, deuterate or salt thereof: 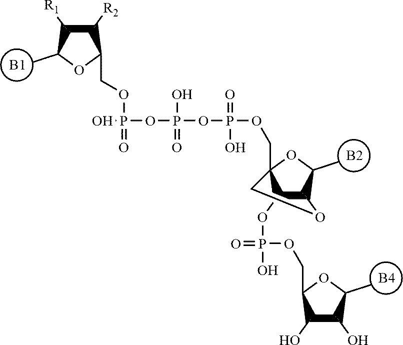
wherein:
ring B1, B2 and B4 are each independently selected from a nucleobase and a modified nucleobase;
R₁ and R₂ are each independently selected from OR₅ and halogen; and
each R₅ is independently selected from H, C₁₋₆ alkyl, C₂₋₆ alkenyl and C₂₋₆ alkynyl, in which C₁₋₆ alkyl, C₂₋₆ alkenyl and C₂₋₆ alkynyl is optionally substituted with one or more of halogen, OH, and/or C₁₋₆ alkoxyl.

12. The compound, stereoisomer, tautomer, deuterate or salt of claim 11, wherein ring B1 and B4 are each independently guanine (G), and ring B2 is each independently adenine (A).

13. The compound, stereoisomer, tautomer, deuterate or salt of claim 11, wherein R₁ and R₂ are each independently selected from OR₅, F, Cl, Br and I, and each R₅ is independently selected from H and C₁₋₆ alkyl, in which C₁₋₆ alkyl is optionally substituted with C₁₋₆ alkoxyl.

14. The compound, stereoisomer, tautomer, deuterate or salt of claim 11, wherein R₁ and R₂ are each independently selected from OH, —O(CH₂)₂OCH₃ and F.

15. A compound, stereoisomer, tautomer, deuterate or salt selected from any of: 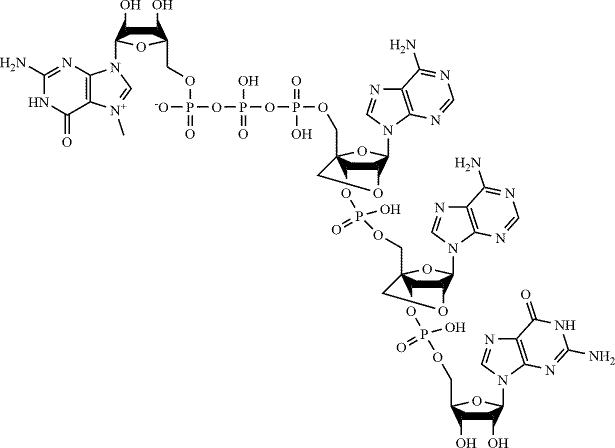 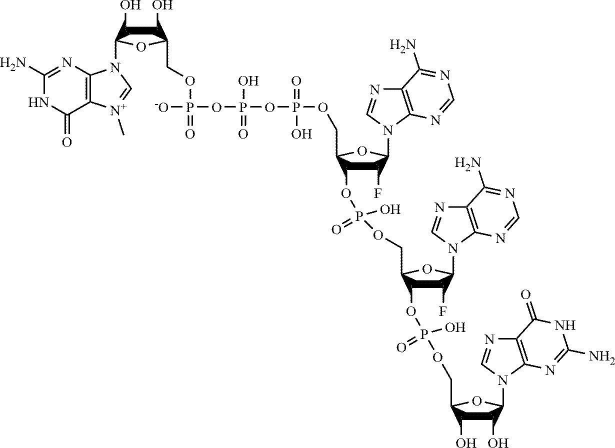 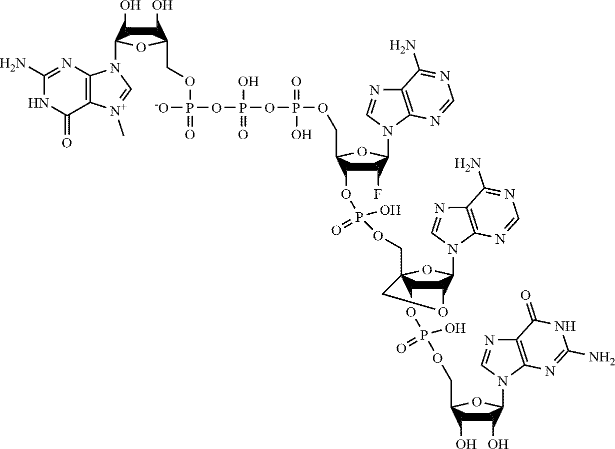 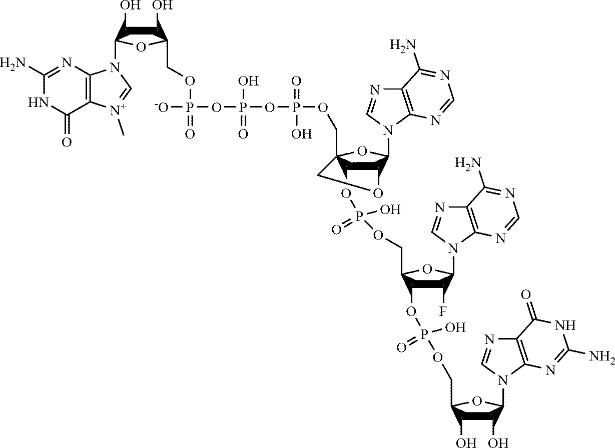 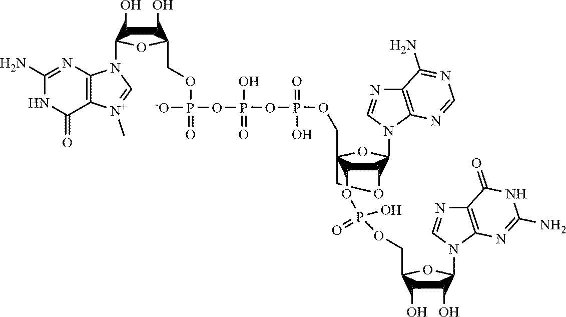 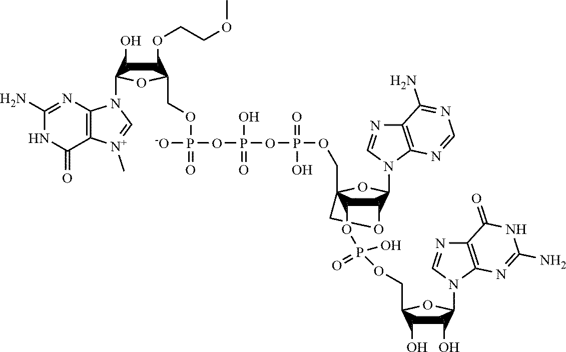 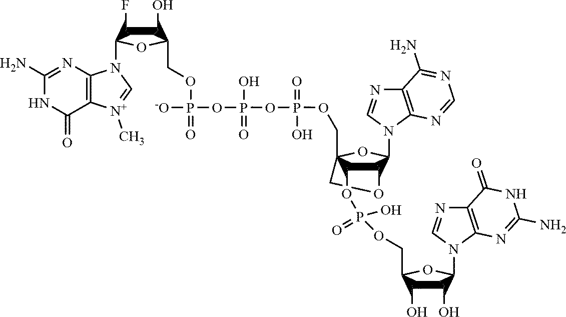 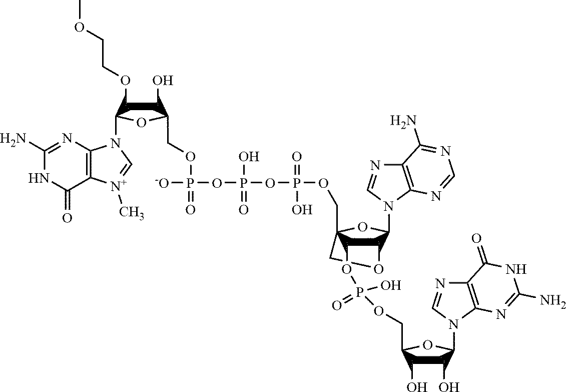 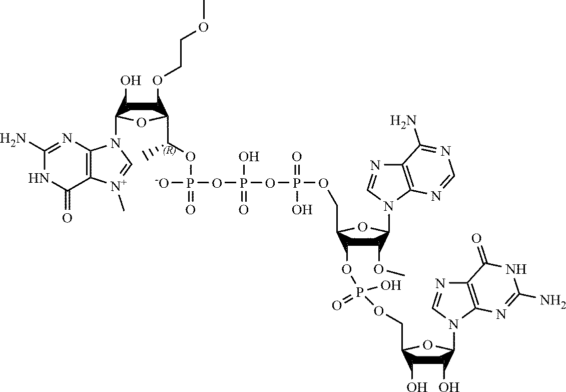
.

16. An RNA molecule whose 5′ end comprises a compound, stereoisomer, tautomer, deuterate or salt of claim 1.

17. The RNA molecule of claim 16, which is an mRNA molecule, and the mRNA molecule contains polynucleotide sequences encoding HPV E6-E7 antigen polypeptides (such as those shown in SEQ ID NO: 1 or SEQ ID NO: 2).

18. A kit for capping an RNA transcript comprising a compound, stereoisomer, tautomer, deuterate or salt of claim 1, and an RNA polymerase.

19. The kit of claim 18, further comprising an RNA molecule.

20. The kit of claim 19, wherein the RNA molecule is an mRNA molecule, and the mRNA molecule contains polynucleotide sequences encoding HPV E6-E7 antigen polypeptides such as those shown in SEQ ID NO: 1 or SEQ ID NO: 2.

21. A method of capping an RNA transcript using a compound, stereoisomer, tautomer, deuterate or salt of claim 1.

22. A method of expressing a protein comprising the step of translating a capped RNA transcript using a compound, stereoisomer, tautomer, deuterate or salt of claim 1.
